(19) United States
(12) Patent Application Publication
(10) Pub. No.: US 2008/0104718 A1
(43) Pub. Date: May 1, 2008

(54) Transgenic Non-Human Animal Models of Ischemia-Reperfusion Injury and Uses Thereof

(75) Inventors: Bradford Berk, Rochester, NY (US); Jun-ichi Abe, Pittsford, NY (US)

Correspondence Address: NIXON PEABODY LLP - PATENT GROUP, 1100 CLINTON SQUARE, ROCHESTER, NY, 14604, US

(73) Assignee: UNIVERSITY OF ROCHESTER, Rochester, NY (US)

(21) Appl. No.: 11/718,677

(22) PCT Filed: Nov. 8, 2005
(86) PCT No.: PCT/US05/40425

§ 371 Date: Jun. 22, 2007

Related U.S. Application Data
(60) Provisional application No. 60/625,881, filed on Nov. 8, 2004.

(51) Int. Cl. A01K 67/00; C12N 5/06; C12N 15/00; A61K 47/00; C12Q 1/02; G01N 33/53; C12N 15/11; C12N 1/20; C12N 7/00; G01N 33/573

(52) U.S. Cl. 800/3; 800/13; 800/18; 435/325; 800/25; 514/789; 435/29; 435/7.21; 800/9; 536/23.2; 435/320.1; 435/252.3; 435/235.1; 435/7.4

(57) ABSTRACT

The present invention relates to a nucleic acid molecule encoding a K94A/K447A mutant of wild type p90 ribosomal S6 kinase (p90RSK) and DNA constructs, expression vectors, and hosts including the mutant p90RSK-encoding molecule. The present invention also relates to two transgenic non-human animal models of ischemic reperfusion (I/R) damage, the first animal having a transgene encoding a mutant p90RSK that is rendered kinase inactive for S703 phosphorylation of NHE1 and the second animal having a transgene encoding for cardiac-specific overexpression of wild type p90RSK in the animal that provides a model for diabetic cardiomyopathy. Also provided are methods for generating transgenic non-human animal models of ischemic reperfusion (I/R) damage; for using the transgenic cells for identifying an agent capable of inhibiting p90RSK-induced I/R damage; for identifying agents that modulate I/R injury resulting from an ischemic event; and for treating individuals to inhibit I/R injury following an ischemic event.

This application claims the benefit of U.S. Provisional Patent Application Ser. No. 60/625,881, filed Nov. 8, 2004, which is hereby incorporated by reference in its entirety.

The subject matter of this application was made with support from the United States Government under National Institutes of Health Grant No. RO1 HL 44721, HL-66919, and GM-071485-01A1. The U.S. Government may have certain rights.

### FIELD OF THE INVENTION

The present invention relates generally to transgenic non-human animal models of ischemic reperfusion damage and the use thereof to identify potential therapeutics for inhibiting reperfusion damage following an ischemic event.

### BACKGROUND OF THE INVENTION

The sodium/hydrogen exchanger (NHE) family regulates intracellular pH (pHi). Among the plasma membrane isoforms only NHE1 is expressed at significant levels in the heart. Numerous experimental studies show that NHE1 activity plays a critical role in acute cardiac ischemia and reperfusion (I/R) injury. Pharmacological strategies that inhibit NHE1 activity dramatically reduce infarct size and improve cardiac function (Karmazyn, M., “Amiloride Enhances Postischemic Ventricular Recovery: Possible Role of Na⁺-H⁺ Exchange,” Am J Physiol 255:H608-615 (1988)). Several compounds, including amiloride, eniporide (EMD-85131), and cariporide (HOE642), are well known as specific NHE1 inhibitors. Using cariporide in an experimental I/R model, infarct size was reduced and cardiac cell death was improved (Miura et al., “Infarct Size Limitation by a New Na⁺-H⁺ Exchange Inhibitor, Hoe 642: Difference From Preconditioning in the Role of Protein Kinase C.,” J Am Coll Cardiol 29:693-701 (1997); Chakrabarti et al., “A Rapid Ischemia-Induced Apoptosis in Isolated Rat Hearts and Its Attenuation by the Sodium-Hydrogen Exchange Inhibitor HOE 642 (Cariporide),” J Mol Cell Cardiol 29:3169-3174 (1997)). This evidence led to the clinical testing of highly selective pharmacological inhibitors of NHE1 as potential therapeutic agents for cardioprotection in acute coronary syndromes and after myocardial infarction. Unfortunately, no clinical benefit was observed in two large clinical trials (Klatte et al., “Increased Mortality After Coronary Artery Bypass Graft Surgery is Associated with Increased Levels of Postoperative Creatine Kinase-Myocardial Band Isoenzyme Release: Results From the GUARDIAN Trial,” J Am Coll Cardiol 38:1070-1077 (2001); Zeymer et al., “The Na⁺/H⁺ Exchange Inhibitor Eniporide as an Adjunct to Early Reperfusion Therapy for Acute Myocardial Infarction. Results of the Evaluation of the Safety and Cardioprotective Effects of Eniporide in Acute Myocardial Infarction (ESCAMI) Trial,” J Am Coll Cardiol 38:1644-1650 (2001)). One reason may be that the basal, acid stimulated homeostatic function of NHE1 is impaired by cariporide and zoniporide, and this function is likely important for cell survival.

It was previously reported that transfection of HEK293 cells with wild-type RSK enhanced NHE phosphorylation and activity, while RSK reduced NHE1 (Takahashi et al., “p90RSK is a Serum-Stimulated NHE Kinase: Regulatory Phosphorylation of Serine 703 of Na⁺/H⁺ Exchanger Isoform-1,” J Biol Chem 274:20206-20214 (1999)). Furthermore, it was found that RSK phosphorylated S703 on the C-terminus of NHE1 and the adapter protein 14-3-3 bound to phospho-S703, which increased NHE1 activity (Lehoux et al., “14-3-3 Binding to Na⁺/H⁺ Exchanger Isoform-1 is Associated With Serum-Dependent Activation of Na⁺/H⁺ Exchange,” J Biol Chem 276:15794-15800 (2001); Cavet et al., “14-3-3beta is a p90 Ribosomal S6 Kinase (RSK) Isoform 1-Binding Protein That Negatively Regulates RSK Kinase Activity,” J Biol Chem 278:18376-18383 (2003)).

Takeishi et al. reported that RSK and ERK1/2 were activated in patients with late phase dilated cardiomyopathy (Takeishi et al., “Activation of Mitogen-Activated Protein Kinases and p90 Ribosomal S6 Kinase in Failing Human Hearts with Dilated Cardiomyopathy,” Cardiovasc Res 53:131-137 (2002)). Also, Seko et al. reported that RSK and ERK1/2 were activated by Raf-1-MAPK cascade in neonatal rat cardiomyocytes stimulated by VEGF (Seko et al., “Vascular Endothelial Growth Factor (VEGF) Activates Raf-1, Mitogen-Activated Protein (MAP) Kinases, and S6 Kinase (p90rsk) in Cultured Rat Cardiac Myocytes,” J Cell Physiol 175:239-246 (1998)). Additionally, RSK and ERK1/2 were activated by Raf-1 stimulation following hypoxia oxygenation in neonatal rat cardiomyocytes (Seko et al., “Hypoxia and Hypoxia/Reoxygenation Activate Raf-1, Mitogen-Activated Protein Kinase, Mitogen-Activated Protein Kinases, and S6 Kinase in Cultured Rat Cardiac Myocytes,” Circ Res 78:82-90 (1996)). Based on these reports, it is proposed herein that NHE1 is activated in the myocardium after I/R by a cascade including ERK1/2, RSK, and NHE1.

The renin-angiotensin and kallikrein-kinin systems are important regulators of blood pressure and atherosclerosis. Renin is an enzyme that converts the circulating substrate angiotensinogen, abundant in many tissues and the circulating blood, into the decapeptide angiotensin I (ang I) in plasma and tissue. Angiotensin-converting enzyme (ACE), present in vascular endothelium, particularly in the lungs, mediates the generation of an octapeptide, angiotensin II (ang II), from angiotensin I. Ang II causes increases in systemic vascular resistance and arterial pressure, which can lead to vasoconstriction, and possibly hypertension. Other cellular reactions mediate by ang II include production of endothelin and superoxide, retention of sodium and water, and cellular proliferation. ACE and ang II inhibitors are well-known post myocardial infarction (MI) therapeutics.

Diabetes is an independent risk factor for both mortality and morbidity after myocardial infarction (Grundy et al., “Diabetes and Cardiovascular Disease: a Statement for Healthcare Professionals From the American Heart Association,” Circulation 100(10):1134-1146 (1999)). A nrber of clinical studies show that post-MI left ventricular function is significantly worse in diabetic patients compared with non-diabetic patients (Zuanetti et al., “Effect of the ACE Inhibitor Lisinopril On Mortality in Diabetic Patients With Acute Myocardial Infarction: Data From the GISSI-3 Study,” Circulation 96(12):4239-4245 (1997); Gustafsson et al., “Effect of the Angiotensin-Converting Enzyme Inhibitor Trandolapril On Mortality and Morbidity in Diabetic Patients With Left Ventricular Dysfunction After Acute Myocardial Infarction,” Trace Study Group J Am Coll Cardiol 34(1):83-89 (1999)). In addition, several clinical studies strongly indicate that activation of the renin-angiotensin system (RAS) in diabetic patients is a critical factor to developing heart failure after MI (Zuanetti et al., “Effect of the ACE Inhibitor Lisinopril On Mortality in Diabetic Patients With Acute Myocardial Infarction: Data From the GISSI-3 Study,” Circulation 96(12):4239-4245 (1997); Gustafsson et al., “Effect of the Angiotensin-Converting Enzyme Inhibitor Trandolapril On Mortality and Morbidity in Diabetic Patients With Left Ventricular Dysfunction After Acute Myocardial Infarction Trace Study Group,” J Am Coll Cardiol 34(1):83-89 (1999)). Although these clinical studies indicated that there is greater benefit for ACE inhibitor treatment post-MI in diabetic patients than nondiabetic patients, the molecular basis for this difference is unclear. Over the past several decades, a number of laboratories have examined the levels and activity of elements of the renin-angiotensin system (RAS) in plasma and in various tissues during diabetes. The measurements of angiotensin (Ang) II and its upstream components of the RAS have been complicated by the rapid degradation of these peptides (Al-Merani et al., “The Half-Lives of Angiotensin II, Angiotensin II-Amide, Angiotensin III, Sar1-Ala8-Angiotensin II and Renin in the Circulatory System of the Rat,” J Physiol 278:471-490 (1978); Chapman et al., “Half-Life of Angiotensin II in the Conscious and Barbiturate-Anaesthetized Rat,” Br J Anaesth 52(4):389-393 (1980)), and the local regulation of this production within specific vascular tissue and lesions (Takai et al., “Induction of Chymase That Forms Angiotensin II in the Monkey Atherosclerotic Aorta,” FEBS Lett 412(1):86-90 (1997)). Therefore, reports on the effects of diabetes on plasma and tissue RAS including ang II levels are controversial (Nakayama et al., “Adrenal Renin-Angiotensin-Aldosterone System in Streptozotocin-Diabetic Rats,” Horm Metab Res 30(1):12-15 (1998); Cronin et al., “Reduced Plasma Aldosterone Concentrations in Randomly Selected Patients With Insulin-Dependent Diabetes Mellitus,” Diabet Med 12(9):809-815 (1995); Price et al., “The Paradox of the Low-Renin State in Diabetic Nephropathy,” J Am Soc Nephrol 10(11):2382-2391 (1999)), and interpretation of these changes is limited by the potential downstream modulation of RAS production and stability.

The importance of PKCP activation during diabetes has been demonstrated by studies reporting that the specific PKCP inhibitor, LY333531, inhibited many abnormalities such as renal mesangial expansion, cardiomyopathy, and monocyte activation in diabetic rats (King et al., “Biochemical and Molecular Mechanisms in the Development of Diabetic Vascular Complications,” Diabetes 3:S105-108 (1996); Tuttle et al., “A Novel Potential Therapy for Diabetic Nephropathy and Vascular Complications: Protein Kinase C beta Inhibition,” Am J Kidney Dis 42(3):456-465 (2003)). It has also been reported that cardiac-specific overexpression of PKCβII, but not PKCε, in transgenic mice decreased cardiac function (Takeishi et al., “Transgenic Overexpression of Constitutively Active Protein Kinase C Epsilon Causes Concentric Cardiac Hypertrophy,” Circ Res 86(12):1218-1223 (2000)). Previously it was shown that H₂O₂-mediated p90RSK activation is partially dependent on PKC activation in Jurkat cells (Abe et al., “Reactive Oxygen Species Activate p90 Ribosomal S6 Kinase Via fyn and ras,” J Biol Chem 275(3):1739-1748 (2000)). Interestingly, p90RSK activation is specifically up-regulated in overexpression of PKCβII transgenic mice, which is thought to be a diabetic cardiomyopathy model (Itoh et al., “Role of p90 Ribosomal S6 Kinase (p90RSK) in Reactive Oxygen Species and Protein Kinase C β (PKC β)-mediated Cardiac Troponin I Phosphorylation,” J Biol Chem 280(25):24135-24142 (2005)).

p90RSK is a serine/threonine kinase, and is involved in activation of nuclear factor-κB by phosphorylation of IK-B (Ghoda et al., “The 90-kDa Ribosomal S6 Kinase (pp90rsk) Phosphorylates the N-terminal Regulatory Domain of IkappaBalpha and Stimulates Its Degradation In Vitro,” J Biol Chem 272(34):21281-21288 (1997)), or phosphorylation of transcription factors, including c-Fos (Chen et al., “Regulation of pp 90rsk Phosphorylation and S6 Phosphotransferase Activity in Swiss 3T3 Cells by Growth Factor-, Phorbol Ester-, and Cyclic AMP-mediated Signal Transduction,” Mol Cell Biol 11(4):1861-1867 (1991)), Nur77 (Fisher et al., “Evidence for Two Catalytically Active Kinase Domains in pp90rsk,” Mol Cell Biol 16(3):1212-1219 (1996)), and CREB (Xing et al., “Coupling of the RAS-MAPK Pathway to Gene Activation by RSK2, a Growth Factor-regulated CREB Kinase,” Science 273(5277):959-963 (1996)). However, the role of p90RSK and its relation with RAS in diabetic hearts remains largely unknown.

What is needed now is a method to treat I/R injury that involves specifically targeting inhibition of RSK and reduction of NHE1 activity in response to agonists such as H₂O₂ and/or other reactive oxygen species, while preserving basal Na⁺/H⁺ exchange function. Such a method would provide a tremendous benefit for prevention of and recovery from myocardial infarction, stroke, and other debilitating and potentially fatal I/R injury-related conditions for which no such treatment currently exists. Also needed is a model for the study of diabetic cardiomyopathy, and a greater understanding of the functional role(s) of p90RSK and PRECE induction in ischemic and diabetic myocardium, which may provide an alternative therapeutic approach to treat diabetic cardiomyopathy.

The present invention is directed to overcoming these and other deficiencies in the art.

### SUMMARY OF THE INVENTION

A first aspect of the present invention relates to a transgenic non-human animal having a transgene encoding a mutant p90 ribosomal S6 kinase (RSK) that is rendered kinase inactive for phosphorylation of NHE1, particularly though not exclusively, phosphorylation at S703. A method of generating the transgenic animal is also disclosed.

A second aspect of the present invention relates to an isolated, recombinant cell comprising a transgene encoding a mutant p90 ribosomal S6 kinase (RSK) that is rendered kinase inactive for phosphorylation of NHE1, particularly though not exclusively, phosphorylation at S703. A method of generating the transgenic animal is also disclosed.

A third aspect of the present invention relates to a method of treating an individual to inhibit reperfusion damage following an ischemic event. This method involves administering to an individual an agent that inhibits p90RSK-induced activation of NHE1, thereby inhibiting activated NHE1-induced reperfusion damage associated with the ischemic event.

A fourth aspect of the present invention relates to a method of identifying an agent capable of inhibiting p90RSK-induced activation of NHE1. This method involves providing a cell culture having cells that express p90RSK and NHE1, treating the cells with a drug to be tested, exposing the cells to an agonist that normally causes RSK-induced activation of NHE1, and determining the level of p90RSK-induced activation of NHE1 in the treated cells A reduction in the level of p90RSK-induced activation of NHE1 occurring in the treated cells, as compared to the untreated cells, indicates the efficacy of the agent.

A fifth aspect of the present invention relates to a method of identifying an agent that modulates ischemic reperfusion (I/R) injury resulting from an ischemic event. This method involves providing a transgenic non-human animal whose genome comprises a transgene encoding a mutant p90 ribosomal S6 kinase (p90RSK) that is rendered kinase inactive for phosphorylation, preferably S703 phosphorylation, of NHEL; exposing the transgenic non-human animal to conditions effective to produce an ischemic event in the transgenic non-human animal; administering to the transgenic non-human animal an agent to be tested; and determining whether the agent modulates the ischemic reperfusion injury resulting from the ischemic event in the transgenic non-human animal (i.e., as compared to a non-human animal lacking the transgene).

A sixth aspect of the present invention relates to an isolated nucleic acid molecule encoding a mutant p90 ribosomal S6 kinase (p90RSIC), where the mutant p90RSK is a K94A/K447A mutant of a wild type p90RSK amino acid sequence. Also provided in the present invention are expression vectors and hosts including a K94A/K447A p90RSK mutant.

A seventh aspect of the present invention relates to a second transgenic non-human animal. This transgenic non-human animal includes a transgene that encodes for cardiac-specific overexpression of wild type p90RSK compared to a non-transgenic animal.

An eighth aspect of the present invention relates to an isolated, recombinant cell comprising a transgene that encodes for cardiac-specific overexpression of wildtype p90RSK.

A ninth aspect of the present invention relates to a method of treating an individual to inhibit ischemia reperfusion injury associated with an ischemic event. This method involves administering to an individual an effective amount of an agent that inhibits p90 ribosomal S6 kinase (p90RSK)-induced activation of pro-renin converting enzyme (PRECE), thereby inhibiting ischemia reperfusion injury associated with an ischemic event.

A tenth aspect of the present invention relates to a method of identifying an agent that modulates ischemic reperfusion injury resulting from an ischemic event. This method involves providing a transgenic non-human animal whose genome comprises a transgene encoding for cardiac-specific overexpression of wild type p90 ribosomal S6 kinase (p90RSK); exposing the transgenic non-human animal to conditions effective to produce an ischemic event in the transgenic non-human animal; administering to the transgenic non-human animal an agent to be tested; and determining whether the agent modulates the ischemic reperfusion (I/R) injury resulting from the ischemic event in the transgenic non-human animal.

The present invention provides two transgenic non-human animals useful for the study of I/R injury and the development of therapeutics and methods of treatment for I/R injury that are directed to new pathological mediators of I/R injury in the heart. Also provided is an improved and much needed method of preventing functional derangement and cell death in cells that have been, or may be, subjected to I/R injury.

### BRIEF DESCRIPTION OF THE DRAWINGS

FIG. 1 is a western blot showing wild type (WT-RSK) and double negative mutant p90 ribosomal S6 kinase (DN-RSK) expression in neonatal rat cardiomyocytes. An adenoviral expression vector containing the DN-RSK gene (Ad.DN-RSK) was transduced into neonatal rat cardiomyocytes. Transduction was for 3 hrs incubated without serum, and cells were harvested after 48 hrs. Cell lysates were prepared and western blot performed with an antibody to RSK that detects both endogenous RSK isoforms (RSK 1 and RSK2) and the transduced DN-RSK.

FIGS. 2A-D are graphs showing that H₂O₂-stimulated intracellular pH (pHi) recovery is inhibited by Ad.DN-RSK. Neonatal rat cardiac myocytes transduced with adenovirus were acid-loaded by NH₄Cl prepulse, plus H₂O₂ treatment for 10 min. Results are average of >10 individual cell recordings. The rate of pHi recovery was measured with BCECF-AM (2′,7′-bis-(2-carboxyethyl)-5-(and-6)-carboxyfluorescein, acetoxymethyl ester). FIG. 2A shows results in Ad.LacZ-transduced cells. FIG. 2B shows the results with Ad.DN-RSK-transduced cells. FIG. 2C shows recovery rate, calculated from the first 60 sec of each recovery curve (n=5). FIG. 2D shows the rate of H⁺ efflux (J_{H}) during pHi recovery, calculated in H₂O₂ stimulated cells. Results are mean ±S.E., n=5,*p<0.05 vs. vehicle-control, †p<0.05 vs. H₂O₂-lacZ.

FIGS. 3A-E show analysis of cardiac RSK expression, endogenous cardiomyocyte RSK phosphorylation and the effect of Ad.DN-RSK on apoptosis (cell death). Endogenous cardiomyocyte RSK phosphorylation was analyzed by western blot analysis using an antibody specific for phospho-RSK (p-RSK). Isolated cardiomyocytes were subjected to A/R (12 hr/10 min). Cell lysates were prepared and subjected to SDS-PAGE (20 μg total protein) followed by western blotting for p-RSK (n=3, *p<0.05). Western blot results are shown in FIG. 3A. FIG. 3B is graph showing increase of p-RSK expression in A/R cells vs. control cells. FIGS. 3C-D are graphs showing effects of Ad.DN-RSK on cell death. Cells were transduced with Ad.LacZ or Ad.DN-RSK for two hr and cultured one day after changing the medium. Apoptosis was induced by 12 hrs anoxia/24 hrs reoxygenation (A/R). FIG. 3C shows quantitation of cardiomyocytes apoptosis performed with a TUNEL (Terminal deoxynucleotidyl Transferase Biotin-dUTP Nick End Labeling) assay. FIG. 3D shows cells death quantitated by anti-DNA fragmentation ELISA. Data are mean ±S.E. (n=5 for each group from 3 independent experiments; *p<0.05). FIG. 3E shows WT-RSK enhanced A/R induced apoptosis in H9c2 cells via NHE1 activity. H9c2 rat embryonic cardiac myoblasts were transduced with cDNAs expressing EGFP alone, WT-RSK, NHE1-WT or NHE1-S703A. The latter three were co-transfected with EGFP to identify transfected cells. Cells were exposed to experimental conditions 48 hrs after transfection. Conditions included EIPA alone (5 μM), A/R (12 hr/24 hr) or both EIPA and A/R. Transfected cells only were counted for analysis and were identified by expression of EGFP. To analyze apoptosis, 100 TUNEL positive cells were measured for each condition. Data are mean ±S.E (n=5 for each group from 3 independent experiments). *p<0.05 vs. Control (no A/R), **p<0.05 vs. A/R, †p<0.05 vs. A/R WT-NHE ††p<0.05 vs. A/R and A/R+RSK.

FIGS. 4A-C show results of treatment consisting of 45 min ischemia/24 hrs reperfusion in non-transgenic littermate controls (NLC) and DN-RSK TG mice. FIG. 4A shows RSK expression detected by western blotting (top panel) and PCR (bottom panel) performed as described in the Examples. FIG. 4B are representative photographs of midventricular myocardium, showing infarct size, from transgenic (TG) DN-RSK mouse and NLC. FIG. 4C is a graph showing quantitation of infarct size (1S) in area at risk (AAR) ratio in NLC (n=11) and DN-RSK TG (n=11, *p<0.05) following treatment as described.

FIGS. 5A-B show a time course of endogenous RSK activation by I/R. Hearts made ischemic by coronary ligation for 45 min followed by the indicated reperfusion times (0, 20, 120, 360 min). After reperfusion, hearts were saline perfused, stained with Evans blue, sectioned, and the ischemic area harvested for western blotting. The phospho-specific p90RSK antibody was used to recognize activated RSK by virtue of binding to phospho-Thr359/Ser363.

FIG. 5A shows the peak of endogenous RSK phosphorylation at 20 min reperfusion. FIG. 5B shows quantitation by densitometry. Results were normalized by arbitrarily setting the baseline value (I/R=0/0) to 1.0 (n=4).

FIGS. 6A-C show results of NHE1 binding to 14-3-3 β in I/R heart tissue. FIG. 6A shows samples from sham and I/R hearts lysed and immunoprecipitated with 14-3-3 β antibody and immunoblotted for NHE1 (upper panel) and 14-3-3 P (middle panel). Total cell lysate was immunoblotted with NHE1 antibody (lower panel). FIG. 6B shows densitometric analysis of NHE1 binding to 14-3-3 after normalizing NLC to 1.0 (n=4), p=0.01). FIG. 6C shows in vitro RSK kinase activity of samples from FIG. 6A.

FIGS. 7A-C are comparisons of DN-RSK-Tg (TG) and control (NLC) hearts after I/R (I=45 min, R=2 wks). FIG. 7A shows H&E (hematoxylin and eosin) and Masson trichrome staining section of mid-ventricular myocardium from TG and NLC mice. FIG. 7B is a fibrosis area measurement from the Masson trichrome staining of FIG. 7A. Values are group means ±S.E, n=11*P<0.05. FIG. 7C shows representative M-mode echocardiographic images of intact beating hearts after reperfusion for 2 weeks, NLC (upper panel) TG (lower panel).

FIGS. 8A-D are western blots of ERK1/2 and PKCα/βII activity in STZ-mediated hyperglycemic mice. FIG. 8A shows result with a PKCα/βII antibody. FIG. 8B shows results with a PKCβII antibody. FIG. 8C shows results with phosphor-specific ERK1/2 antibody. FIG. 8D shows results with anti-ERK1/2. These results demonstrate that PKCα/βII and p90RSK activation, but not ERK1/2, were increased in STZ-mediated hyperglycemic mice.

FIG. 9 is a graph showing p90RSK activation in STZ-mediated hyperglycemic mice. p90RSK activity was detected by in vitro kinase assay using S6 kinase substrate peptide as described below. Data (n=3) were expressed as mean ±S.D. **p<0.01

FIGS. 10A-B are immunoblots of lysates prepared from 10-week-old NLC and WT-p90RSK-Tg mice hearts showing the cardiac selective expression of WT-p90RSK. FIG. 10A shows results using a p90RSK antibody.

FIG. 10B shows actin control on same lysates.

FIGS. 11A-D show effects of ischemia on cardiac function and enzyme production. FIG. 11A are measurements of left ventricular developed pressure before, during, and after global (no-flow) ischemia followed by reperfusion. FIG. 11B are measurements of left ventricular dP/dtmax before, during, and after global (no-flow) ischemia followed by reperfusion. All experimental values calculated for NLC (n=5) and WT-p90RSK-Tg hearts (n=5) are represented as mean ±S.D. FIGS. 11C-D shows creative kinase (CK) and lactate dehydrogenase (LDH) cardiac enzymes, respectively, measured in the superfusate from the heart after ischemia (n=4) and reported as mean units/L±S.D.

FIGS. 12A-B are protein expression profiles of NLC and WT-p90RSK-Tg mice hearts. FIG. 12A upper and lower panels, are 2-D gels of NLC (upper) and WT-p90RSK-Tg (lower) cardiac proteins, stained with silver staining; IPG NL 4-7; 10% SDS-PAGE. After staining with silver staining, gel images were compared. Spots were selected that were significantly increased in WT-p90RSK-Tg samples, and digested with trypsin, then analyzed with MALDI-TOF mass spectrometry. Analysis of MALDI-TOF mass spectrometry demonstrates the 40% matching with PRECE-2 (mKLK26) amino acid sequence (SEQ ID NO: 12), shown in FIG. 12B. Bold characters in mouse PRECE-2 amino acid sequence indicate matched amino acids.

FIGS. 13A-B show PRECE expression in INT-p90RSH-Tg vs. NLC mice.

FIG. 13A shows results of relative quantitative RT-PCR analysis, showing PRECE mRNA expression increased in WT-p90RSK-Tg mice hearts. 18S rRNA was used as internal control. FIG. 13B is densitometric analysis of PRECE mRNA expression in NLC and WT-p90RSK-Tg mouse hearts. Results were normalized for all experiments by arbitrary setting the mean densitometry of NLC heart samples to 1.0 (shown in mean ±S.D., n=3, **p<0.01).

FIGS. 14A-B are analysis of angiotensinogen level in NLC and WT-p90RSK-Tg mice after perfusion. FIG. 14A shows immunoblot of lysates prepared from 10-week-old NLC and WT-p90RSK-Tg mice hearts and contacted with angiotensinogen (upper panel) and tubulin (bottom panel) antibodies. FIG. 14B shows densitometric analysis of serial angiotensinogen protein level in NLC and WT-p90RSK-Tg mouse hearts after perfusion. Results were normalized for all experiments by arbitrary setting the mean densitometry of NLC heart samples to 1.0 at 3 min after KH buffer perfusion (shown in mean ±S.D., n=4, *p<0.01).

FIGS. 15A-B show diabetes-mediated PRECE mRNA expression inhibited in DN-p90RSK-Tg mouse hearts. FIG. 15A shows STZ injection-mediated diabetes increased PRECE mRNA expression after 2 weeks of STZ injection, which was inhibited in DN-p90RSK-Tg mouse hearts. 18S rRNA was used as internal control. FIG. 15B is densitometric analysis of PRECE mRNA expression in STZ-injected diabetic NLC and DN-p90RSK-Tg mice. Results were normalized for all experiments by arbitrary setting the densitometry of control heart samples to 1.0 (shown in mean ±S.D., n=4, *p<0.05).

FIGS. 16A-H demonstrate ACE inhibitor (captopril 50 μM) protected WT-p90RSK-Tg hearts but not NLC hearts from I/R-induced contractile dysfunction. FIGS. 16A-D show measurements of left ventricular developed pressure and dP/dtmax before, during, and after global (no-flow) ischemia followed by reperfusion with vehicle or captopril (50 μM) pretreatment in NLC hearts. Short 20 min (FIG. 16A-B) or prolonged 40 min (FIG. 16C-D) ischemia was performed. FIGS. 16E-F shows measurements of left ventricular developed pressure and dP/dtmax, respectively, before, during, and after global (no-flow) ischemia followed by reperfusion with vehicle or captopril (50 μM) pretreatment in WT-p90RSK-Tg mouse hearts after 20 min ischemia. FIGS. 16G-H show measurements of left ventricular developed pressure and dP/dtmax, respectively, after prolonged 40 min (FIG. 16G) ischemia in NLC hearts and short 20 min (FIG. 16H) ischemia in WT-p90RSK-Tg hearts followed by 25 min reperfusion with vehicle or captopril (50 μM) pretreatment (shown in mean ±S.D., n=5, **p<0.01).

FIGS. 17A-B demonstrate ACE inhibitor (captopril 50 μM) protected WT-p90RSK-Tg hearts but not NLC hearts from I/R-induced cardiac injury. Cardiac enzymes were measured in the superfusate from the NLC hearts after prolonged 40 min ischemia (n=4) and p90RSK-Tg mouse hearts after short 20 min ischemia (n=4). FIG. 17A shows results of creatine kinase (CK) release.

FIG. 17B shows results of lactate dehydrogenase (LDH) release values reported as mean units/L ±S.D. (*p<0.05, **p<0.01).

FIGS. 18A-C are hemodynamic measurements in NLC (n=6) and WT-p90RSK-Tg (n=6) mice at age of 10 months old. All data are expressed as mean ±S.D. (**p<0.01, *p<0.05).

FIG. 19 are representative M-mode echocardiographic images of contracting hearts in 10 months old NLC and WT-p90RSK-Tg mice, showing cardiac dysfunction in WT-p90RSK-Tg mice.

FIGS. 20A-B shows percent fractional shorting (% FS) and velocity of circumferential fiber shortening (Vcfs), respectively, in 3 and 10 months old NLC (n=6), and WT-p90RSK-Tg (n=5) mice. Values (mean ±SEM) were determined by echocardiography. **p<0.01 between groups.

FIGS. 21A-C show detection of apoptosis by TUNEL assay. FIG. 21A shows results with NLC mice. FIG. 21B shows results with WT-p90RSK-Tg mice. Green fluorescence shows apoptotic cardiomyocytes stained with TUNEL, nuclei were counterstained with Hoechst33342 staining (blue), and cardiomyocytes were stained with anti-α-actin (sarcomeric) (clone EA-53, red). Overlay images were shown. FIG. 21C is quantitative analysis of apoptotic cells. The vertical axis indicates the % ratio of TUNEL-positive cell number relative to that of Hoechst33342-positive nuclei, which were clearly overlaid with EA-53 staining (indicated by arrows). Cells which did not counter stained clearly with EA-53 staining (indicated by asterisk) were not counted. More than 1000 cells were screened per section.

FIG. 22 shows Bcl-2 expression in NLC and WT-p90RSK-Tg mice. Lysates were prepared from 10-months-old NLC and WT-p90RSK-Tg mice hearts and immunoblot with a Bcl-2 (upper panel) and actin (lower panel) antibodies.

FIG. 23 shows ratios of heart weight to body weight (HW/BW) in 3 and 10 months old NLC and WT-p90RSK-Tg mice. Results demonstrate increase in cardiac hypertrophy over time.

FIG. 24A-B are blots showing atrial natriuretic factor (ANF) and brain natriuretic protein respectively (BNP). The upper panels in FIGS. 24A-B show mRNA expression in 10 months old NLC and WT-p90RSK-Tg mice. ANF and BNP mRNA levels were determined by relative quantitative RT-PCR. 18S rRNA was used as internal control. FIG. 24A-B, bottom panel, shows densitometric analysis of ANF and BNP mRNA expression, as marked. Results were normalized for all experiments by arbitrary setting the densitometry of NLC 10 months old heart samples to 1.0 (shown in mean ±S.D., n=4, **p<0.01).

FIG. 25 is representative image of NLC and WT-p90RSK-Tg hearts at 10 months of age.

FIGS. 26A-B are histological images (at 200×, Masson's trichrome) of hearts from a NLC and WT-p90RSK-Tg, respectively, at 10 months old, indicating interstitial fibrosis with apoptosis in WT-p90RSH-Tg mice.

FIGS. 27A-E demonstrate AT1 receptor blocker (olmesartan 10 μM) protected WT-p90RSK-Tg but not NCL hearts from I/R-induced contractile dysfunction. FIGS. 27A-B are graphs of measurements of left ventricular developed pressure and dP/dtmax, respectively, before, during, and after global (no-flow) ischemia followed by reperfusion with vehicle or olmesartan (an AT 1 receptor blocker) (10 μM) pretreatment in NLC hearts. Prolonged 40 min ischemia was performed. FIGS. 27C-D are graphs of measurements of left ventricular developed pressure and dP/dtmax, respectively, before, during, and after global (no-flow) ischemia followed by reperfusion with vehicle or olmesartan (10 μM) pretreatment in WT-p90RSK-Tg mouse hearts after 20 min ischemia. FIG. 27E is a graph of the measurement of left ventricular developed pressure after prolonged 40 min ischemia in NLC hearts and short 20 min ischemia in WT-p90RSK-Tg hearts followed by 25 min reperfusion with vehicle or olmesartan pretreatment (shown in mean ±S.D., n=5, **p<0.01).

FIG. 28 is a VISTA plot of the mouse KLK26 (PRECE-2) region (chromosome7; 38,077,009-38,091,292) on human genome (chromosome19: 56,049,788-56,073,634) detailing conserved regions between human and mouse. Peaks represent conserved regions, peak width represents the size of the conserved region, and peak height represents the percentage identity between human and mouse sequences. The positions of the exons are indicated by the blue boxes above the upper axis. The shaded regions indicate the conserved regions with the identity above 75%.

### DETAILED DESCRIPTION OF THE INVENTION

Applicants have identified the role that p90 ribosomal S6 Kinase (RSK or p90RSK, which are used interchangeably herein) plays in the activation of NHE1. In particular, applicants have demonstrated that inhibiting RSK activation of NHE1 can minimize ischemic-reperfusion injury while not otherwise modifying basal NHE1 exchange activity.

One aspect of the present invention relates to a method of (i.e., an assay for) identifying an agent (e.g., a drug) capable of inhibiting p90RSK-induced activation of NHE1. This method involves providing a cell culture having cells that express RSK and NHE1, treating the cells with an agent to be tested, exposing the cells to an agonist that normally causes RSK-induced activation of NHE1, and determining the level of RSK-induced activation of NHE1 in the treated cells. A reduction in the level of RSK-induced activation of NHE1 occurring in the treated cells, as compared to untreated cells exposed to the same agonist, indicates efficacy of the agent.

In one embodiment of this assay, exposure to the agonist precedes treatment of the cells in culture with the agent to be tested.

In another embodiment, the assay involves exposing the cells in culture to an agonist after treating the cells with the drug to be tested.

In yet another embodiment, the assay can be carried out with exposure to the agonist and treatment of the cells with the agent being performed concurrently.

In all aspects of this assay, the cells may be exposed to an agonist. This can be carried out by directly or indirectly by adding a reactive oxygen species to the cell culture. Suitable reactive oxygen species include, without limitation, H₂O₂, a molecule that generates H₂O₂, or any other reactive oxygen species.

Determining the level of p90RSK-induced activation of NHE1 in the treated cells may be carried out by any suitable method known in the art, including, without limitation, measuring H⁺ efflux from the cells, measuring the binding of 14-3-3 proteins to NHE1 in the cells, measuring the S703 phosphorylation or dephosphorylation of NHE1 in the cells (e.g., using an antibody specific to phosphorylated or dephosphorylated NHE1 S703), measuring the changes in intracellular pH in the cells, measuring the changes in sodium fluxes in the cells, as well as any combination thereof.

Cells suitable for use in the cell culture of this aspect of the present invention are any cells that undergo functional derangement and cell death in response to ischemia/reperfusion, reactive oxygen species or oxidative stress, including, without limitation, cardiac muscle cells, smooth muscle cells, skeletal muscle cells, neuronal cells, and other cell types where reactive oxygen species, ischemia/reperfusion, and oxidative stress contribute to tissue dysfunction, cell impairment, and cell death. Preferably such cells are mammalians cells, including, without limitation, rodent and human.

The present invention also relates to a method of treating an individual to inhibit reperfusion damage following an ischemic event. This method involves administering to an individual an agent that inhibits p90RSK-induced activation of NHE1, thereby inhibiting activated NHE1-induced reperfusion damage associated with the ischemic event. In this aspect of the present invention, the agent that is administered preferably inhibits RSK-induced activation of NHE1 selectively, without altering basal Na⁺/H⁺ exchange activity in the subject.

As described in greater detail herein below, pharmacological and genetic studies indicate that the Na⁺/H⁺ exchanger isoform 1 (NHE1) plays a critical role in myocardial ischemia and reperfusion (I/R) injury. p90RSK phosphorylates the serine at position 703 of NHE1, stimulating the binding of NHE1 to the 14-3-3 protein, which, in turn, activates NHE1, leading to functional degradation and ultimately to apoptosis (cell death) of the NHE1-activated cells. Because the I/R injury results from a series of steps, I/R-mediated injury, i.e., reperfusion damage following an ischemic event, can be prevented or ameliorated by inhibiting the ability of RSK to phosphorylate NHE1, by decreasing the level of phosphorylation that NHE1 undergoes, or by interfering with the binding of the 14-3-3 protein with NHE1. As used herein “inhibition of RSK-induced activation of NHE1” is intended to mean the inhibition of the step of activating NHE1 as well as interfering with maintenance or function of the activated NHE1. Therefore, in one embodiment, the method of treating an individual to inhibit reperfusion damage following an ischemic event involves administering an agent that inhibits RSK phosphorylation of NHE1 S703. In another embodiment, this method involves administering an agent that accelerates the dephosphorylation of NHE1 S703. In yet another embodiment, this method involves administering an agent that accelerates the dissociation of a 14-3-3 protein from phosphorylated NHE1 S703.

Ischemic events suitable for treatment according to the present invention include, without limitation, heart attack (myocardial infarction), acute coronary syndrome, coronary artery bypass surgery, stroke, gastrointestinal ischemia, peripheral vascular disease, and surgical procedures associated with tissue ischemia.

All mammals are suitable individuals for treatment using this method of the present invention. Exemplary mammals include humans, non-human primates, rodents such as mice, rats, and guinea pigs, dogs, cats, etc.

In all aspects of this method of the present invention, suitable methods of “administering” the agent include, without limitation, oral, intradermal, intramuscular, intraperitoneal, intravenous, subcutaneous, or intranasal. Preferred routes of administration deliver the active agent (e.g. drug) directly to the site of the ischemic event, thereby regulating the activation of NHE1 within the affected tissues. The agents may be administered alone or with suitable pharmaceutical carriers, and can be in solid or liquid form such as, tablets, capsules, powders, solutions, suspensions, or emulsions.

The active compounds of the present invention may be orally administered, for example, with an inert diluent, or with an assimilable edible carrier, or they may be enclosed in hard or soft shell capsules, compressed into tablets, or incorporated directly with the food of the diet. For oral therapeutic administration, the agents of the present invention may be incorporated with excipients and used in the form of tablets, capsules, elixirs, suspensions, syrups, and the like. The amount of active compound in such therapeutically useful compositions is such that a suitable dosage will be obtained.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

These active compounds may also be administered parenterally. Solutions or suspensions of these active compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

In all aspects of this method, administration of the agent of the present invention may occur at the time of presentation of the ischemic event (i.e., soon after its occurrence), prior to presentation of the ischemic event, or concurrently with the ischemic event. In addition, such administration can be carried out in combination with other known therapeutic agents or hereafter developed therapeutic agents for the treatment of the ischemic event.

The present invention also relates to a transgenic non-human animal having a transgene encoding a mutant p90RSK that is rendered kinase inactive for cellular substrates including, without limitation, serine 703 (S703) phosphorylation of NHE1. According to one embodiment, the transgenic non-human animal is bred to contain both somatic and germ cells that harbor the RSK mutant transgene. In another embodiment, the transgenic non-human animal of the present invention is a somatic mosaic (i.e, harbors the RSK mutation in a subpopulation of somatic cells that have been transformed so as to express the transgene).

As used herein, kinase inactive forms of p90RSK are those that exhibit less than 25% activity (as compared to the rat p90RSK of SEQ ID NO:1) preferably less than 10% activity, more preferably less than 5% activity (including complete absence of activity).

Regardless of the embodiment, the transgenic non-human animal of the present invention is prepared so as to express the mutant p90RSK protein in one or more of cardiac muscle cells, smooth muscle cells, skeletal muscle cells, neuronal cells, and other cell types where reactive oxygen species, ischemia/reperfusion, and oxidative stress contribute to tissue dysfunction, cell impairment, and cell death.

In one aspect of the present invention the transgene is inserted into a suitable vector under the control of a tissue-specific nucleic acid promoter. An exemplary promoter is the α-myosin heavy chain promoter region (α-MHC), which allows expression preferentially in myosin-containing tissues, e.g., in the heart.

The term transgenic animal refers to an animal in which there has been a deliberate modification of the genome, i.e, the material responsible for inheritance. Foreign DNA is introduced into the animal, using recombinant DNA technology, and then must be transmitted through the germ line so that every cell, including germ cells, of the animal contains the same modified genetic material. The application of targeted gene modification and production of transgenic animals is a powerful tool for studying gene function in the context of a whole animal. Transgenic animals can be created by several methods that include either microinjection or viral infection of embryos, or through the manipulation in culture of embryonic stem cells that are subsequently incorporated back into the embryo for insertion into the germ line. Any of these techniques is useful for altering the expression of endogenous proteins by transfer of recombinant genes into cells in culture and into live animals to produce transgenic animals harboring the desired gene (Evans, M. J., “Potential for Genetic Manipulation of Mammals,” Mol Biol Med 6:557-565 (1989); Mansour, S. L., “Gene Targeting in Murine Embryonic Stem Cells: Introduction of Specific Alterations into the Mammalian Genome,” Genet Anal Tech Appl 7:219-227 (1990), which are hereby incorporated by reference in their entirety).

The transgenic non-human animal of the present invention may be made, for example, by DNA microinjection (Gordon et al., “Integration and Stable Germ Line Transformation of Genes injected into Mouse Pronuclei,” Science 214:1244-1246 (1981), which is hereby incorporated by reference in its entirety), a method used initially for mice, but has since been applied to many animal species. Briefly, this method involves the direct microinjection of a chosen gene construct (a single gene or a combination of genes) from another member of the same species or from a different species, into the pronucleus of a fertilized ovum. Microinjection of nucleic acid molecules into fertilized eggs (pronuclear stage) can be carried using an inverted microscope, micromanipulation equipment, and injection/holding devices. The pronuclear microinjection method of producing a transgenic animal results in the introduction of DNA sequences into the chromosomes of the fertilized eggs. The animal arising from the injected egg will carry the new gene and subsequently transmit this gene and its effect to offspring. If this transferred genetic material is integrated into one of the embryonic chromosomes, the animal will be born with a copy of this new information in every cell. The modified nucleic acid molecule must be integrated into the genome prior to the doubling of the genetic material that precedes the first cleavage. If this does not occur, only a few cells will integrate the gene. Because the germline of mammals is well protected against the incorporation of foreign genetic material, early embryonic stages (i.e., before the cells differentiate into the precursors of body and germ cells) are best suited for genetic manipulation. For this reason, the desired nucleic acid molecule is introduced into the fertilized egg at the earliest stage, which is the pronuclear period immediately following fertilization. The microinjected eggs are placed into a foster recipient and a normal pregnancy ensues.

Some of the resulting offspring animals in the litter will be somatic mosaics, in that a fraction of their somatic (body) cells will be hemizygous (have only one copy of the desired modified/mutated gene). These animals are identified, for example, by using polymerase chain reaction (PCR) for detection of the transgene. A fraction of the animals in this group will also be mosaic in their germ lines, which is determined by testing for progeny that are purely hemizygous. Chimeric offspring purely hemizygous for the desired trait are then mated to obtain homozygous individuals, and colonies characterized by the presence of the desired mutant protein are established.

In accordance with the invention, a nucleic acid molecule encoding a mutant RSK protein of the present invention is introduced in vivo using microinjection techniques, as describe above, and in Example 1, below, to produce a transgenic DN-RSK mutant non-human animal.

In one embodiment of the present invention, the transgenic non-human animal of the present invention is a somatic mosaic (i.e, harbors the RSK transgene of choice in a subpopulation of somatic cells only). In this aspect, the transgenic animal is prepared using standard DNA transformation techniques to incorporate the RSK mutant or wild type nucleic acid molecule into the sornatic cells of the animal. This involves, briefly, adding the desired nucleic acid molecule to cells other than egg or sperm cells. This can be carried out by preparing the desired RSK mutation nucleic acid molecule, combining it with suitable regulatory nucleic acid molecules, and inserting it into a host animal using any number of suitable methods. Recombinant molecules can be introduced into cells, without limitation, via direct injection of “naked” DNA into the animal using, e.g., electroporation or by gene gun; or incorporation into the host animal using viral vectors (transduction) or liposomal vectors containing the desired RSK mutant nucleic acid molecule, or using any other methods known in the art (e.g., as described by Sambrook et al., Molecular Cloning: A Laboratory Manual, Second Edition, Cold Springs Laboratory, Cold Springs Harbor, N.Y. (1989), which is hereby incorporated by reference in its entirety).

Suitable hosts are all non-human mammals, including, without limitation, rodents, such as mice or rats, as well as those identified above.

In one aspect of the present invention the transgenic non-human animal contains a nucleic acid molecule encoding a p90RSK mutant protein. A “p90RSK mutant” as used herein means a protein or polypeptide wherein specific amino acid substitutions to the mature wild-type RSK protein have been made that render the protein substantially inactive (preferably fully inactive) for kinase activity toward the ribosomal protein S6 peptide. “Wild-type RSK,” as used herein means a RSK protein or variant thereof, including but not limited to, that of rat, mouse, or human (e.g., SEQ. ID. No. 3; GenBank Accession No. M99169; Swiss-Pro Accession No. P18653; GenBank Accession No AF090421) that retains at least 75%, preferably 85-115%, more preferably 95-100% of normal activity. In a preferred embodiment of the present invention, the RSK mutant contains two separate amino acid substitutions, namely, a lysine to alanine substitution at peptide 94 (K94A) and a lysine to alanine substitution at peptide 447 (K447A) of the native RSK polypeptide, making the preferred K94A/K447A RSK mutant of the present invention, which is inactive for cellular substrates including serine 703. In one aspect of the present invention, the mutant p90RSK protein is a rat protein, made by selected amino acid substitutions made to the wild type rat p90RSK-1 (R. norvegicus, sp:Q63531—K6A1_RAT Ribosomal protein S6 kinase alpha 1), SEQ ID NO: 1, as follows:

```
Met Pro Leu Ala Gln Leu Lys Glu Pro Trp Pro Leu Met Glu Leu Val
    1 5 10 15
Pro Leu Asp Pro Glu Asn Gly Gln Ala Ser Gly Glu Glu Ala Gly Leu
                          20 25 30
Gln Pro Ser Lys Asp Glu Gly Ile Leu Lys Glu Ile Ser Ile Thr His
                  35 40 45
His Val Lys Ala Gly Ser Glu Lys Ala Asp Pro Ser His Phe Glu Leu
          50 55 60
Leu Lys Val Leu Gly Gln Gly Ser Phe Gly Lys Val Phe Leu Val Arg
  65 70 75 80
Lys Val Thr Arg Pro Asp Asn Gly His Leu Tyr Ala Met Lys Val Leu
                                  85 90 95
Lys Lys Ala Thr Leu Lys Val Arg Asp Arg Val Arg Thr Lys Met Glu
                        100 105 110
Arg Asp Ile Leu Ala Asp Val Asn His Pro Phe Val Val Lys Leu His
                115 120 125
Tyr Ala Phe Gln Thr Glu Gly Lys Leu Tyr Leu Ile Leu Asp Phe Leu
        130 135 140
Arg Gly Gly Asp Leu Phe Thr Arg Leu Ser Lys Glu Val Met Phe Thr
145 150 155 160
Glu Glu Asp Val Lys Phe Tyr Leu Ala Glu Leu Ala Leu Gly Leu Asp
                                165 170 175
His Leu His Ser Leu Gly Ile Ile Tyr Arg Asp Leu Lys Pro Glu Asn
                        180 185 190
Ile Leu Leu Asp Glu Glu Gly His Ile Lys Leu Thr Asp Phe Gly Leu
                195 200 205
Ser Lys Glu Ala Ile Asp His Glu Lys Lys Ala Tyr Ser Phe Cys Gly
        210 215 220
Thr Val Glu Tyr Met Ala Pro Glu Val Val Asn Arg Gln Gly His Thr
225 230 235 240
His Ser Ala Asp Trp Trp Ser Tyr Gly Val Leu Met Phe Glu Met Leu
                                245 250 255
Thr Gly Ser Leu Pro Phe Gln Gly Lys Asp Arg Lys Glu Thr Met Thr
                        260 265 270
Leu Ile Leu Lys Ala Lys Leu Gly Met Pro Gln Phe Leu Ser Thr Glu
                275 280 285
Ala Gln Ser Leu Leu Arg Ala Leu Phe Lys Arg Asn Pro Ala Asn Arg
        290 295 300
Leu Gly Ser Gly Pro Asp Gly Ala Glu Glu Ile Lys Arg His Ile Phe
305 310 315 320
Tyr Ser Thr Ile Asp Trp Asn Lys Leu Tyr Arg Arg Glu Ile Lys Pro
                                325 330 335
Pro Phe Lys Pro Ala Val Ala Gln Pro Asp Asp Thr Phe Tyr Phe Asp
                        340 345 350
Thr Glu Phe Thr Ser Arg Thr Pro Arg Asp Ser Pro Gly Ile Pro Pro
                355 360 365
Ser Ala Gly Ala His Gln Leu Phe Arg Gly Phe Ser Phe Val Ala Thr
        370 375 380
Gly Leu Met Glu Asp Asp Ser Lys Pro Arg Ala Thr Gln Ala Pro Leu
385 390 395 400
His Ser Val Val Gln Gln Leu His Gly Lys Asn Leu Val Phe Ser Asp
                                405 410 415
Gly Tyr Ile Val Lys Glu Thr Ile Gly Val Gly Ser Tyr Ser Val Cys
                        420 425 430
Lys Arg Cys Val His Lys Ala Thr Asn Met Glu Tyr Ala Val Lys Val
                435 440 445
Ile Asp Lys Ser Lys Arg Asp Pro Ser Glu Glu Ile Glu Ile Leu Leu
        450 455 460
Arg Tyr Gly Gln His Pro Asn Ile Ile Thr Leu Lys Asp Val Tyr Asp
465 470 475 480
Asp Ser Lys His Val Tyr Leu Val Thr Glu Leu Met Arg Gly Gly Glu
                                485 490 495
Leu Leu Asp Lys Ile Leu Arg Gln Lys Phe Phe Ser Glu Arg Glu Ala
                        500 505 510
Ser Phe Val Leu Tyr Thr Ile Ser Lys Thr Val Glu Tyr Leu His Ser
                515 520 525
Gln Gly Val Val His Arg Asp Leu Lys Pro Ser Asn Ile Leu Tyr Val
        530 535 540
Asp Glu Ser Gly Asn Pro Glu Cys Leu Arg Ile Cys Asp Phe Gly Phe
545 550 555 560
Ala Lys Gln Leu Arg Ala Glu Asn Gly Leu Leu Met Thr Pro Cys Tyr
                                565 570 575
Thr Ala Asn Phe Val Ala Pro Glu Val Leu Lys Arg Gln Gly Tyr Asp
                        580 585 590
Glu Gly Cys Asp Ile Trp Ser Leu Gly Val Leu Leu Tyr Thr Met Leu
                595 600 605
Ala Gly Tyr Thr Pro Phe Ala Asn Gly Pro Ser Asp Thr Pro Glu Glu
        610 615 620
Ile Leu Thr Arg Ile Ser Ser Gly Lys Phe Thr Leu Ser Gly Gly Asn
625 630 635 640
Trp Asn Thr Val Ser Glu Thr Ala Lys Asp Leu Val Ser Lys Met Leu
                                645 650 655
His Val Asp Pro His Gln Arg Leu Thr Ala Lys Gln Val Leu Gln His
                        660 665 670
Pro Trp Ile Thr Gln Lys Asp Lys Leu Pro Gln Ser Gln Leu Ser His
                675 680 685
Gln Asp Leu Gln Leu Val Lys Gly Gly Met Ala Ala Thr Tyr Ser Ala
        690 695 700
Leu Ser Ser Ser Lys Pro Thr Pro Gln Leu Lys Pro Ile Glu Ser Ser
705 710 715 720
Ile Leu Ala Gln Arg Arg Val Arg Lys Leu Pro Ser Thr Thr Leu
                                725 730 735
```

This amino acid is encoded by the nucleotide sequence for Rat S6 protein kinase (RSK-1), which sequence is available at GenBank Accession No. M19969, and has SEQ ID NO: 3, shown herein below.

An exemplary mutant RSK of the present invention is the K94A/K447A RSK mutant, having an amino acid sequence of SEQ ID NO: 2 as follows:

```
Met Pro Leu Ala Gln Leu Lys Glu Pro Trp Pro Leu Met Glu Leu Val
    5 10 15 15
Pro Leu Asp Pro Glu Asn Gly Gln Ala Ser Gly Glu Glu Ala Gly Leu
                          20 25 30
Gln Pro Ser Lys Asp Glu Gly Ile Leu Lys Glu Ile Ser Ile Thr His
                  35 40 45
His Val Lys Ala Gly Ser Glu Lys Ala Asp Pro Ser His Phe Glu Leu
          50 55 60
Leu Lys Val Leu Gly Gln Gly Ser Phe Gly Lys Val Phe Leu Val Arg
  65 70 75 80
Lys Val Thr Arg Pro Asp Asn Gly His Leu Tyr Ala Met Ala Val Leu
                                  85 90 95
Lys Lys Ala Thr Leu Lys Val Arg Asp Arg Val Arg Thr Lys Met Glu
                        100 105 110
Arg Asp Ile Leu Ala Asp Val Asn His Pro Phe Val Val Lys Leu His
                115 120 125
Tyr Ala Phe Gln Thr Glu Gly Lys Leu Tyr Leu Ile Leu Asp Phe Leu
        130 135 140
Arg Gly Gly Asp Leu Phe Thr Arg Leu Ser Lys Glu Val Met Phe Thr
145 150 155 160
Glu Glu Asp Val Lys Phe Tyr Leu Ala Glu Leu Ala Leu Gly Leu Asp
                                165 170 175
His Leu His Ser Leu Gly Ile Ile Tyr Arg Asp Leu Lys Pro Glu Asn
                        180 185 190
Ile Leu Leu Asp Glu Glu Gly His Ile Lys Leu Thr Asp Phe Gly Leu
                195 200 205
Ser Lys Glu Ala Ile Asp His Glu Lys Lys Ala Tyr Ser Phe Cys Gly
        210 215 220
Thr Val Glu Tyr Met Ala Pro Glu Val Val Asn Arg Gln Gly His Thr
225 230 235 240
His Ser Ala Asp Trp Trp Ser Tyr Gly Val Leu Met Phe Glu Met Leu
                                245 250 255
Thr Gly Ser Leu Pro Phe Gln Gly Lys Asp Arg Lys Glu Thr Met Thr
                        260 265 270
Leu Ile Leu Lys Ala Lys Leu Gly Met Pro Gln Phe Leu Ser Thr Glu
                275 280 285
Ala Gln Ser Leu Leu Arg Ala Leu Phe Lys Arg Asn Pro Ala Asn Arg
        290 295 300
Leu Gly Ser Gly Pro Asp Gly Ala Glu Glu Ile Lys Arg His Ile Phe
305 310 315 320
Tyr Ser Thr Ile Asp Trp Asn Lys Leu Tyr Arg Arg Glu Ile Lys Pro
                                325 330 335
Pro Phe Lys Pro Ala Val Ala Gln Pro Asp Asp Thr Phe Tyr Phe Asp
                        340 345 350
Thr Glu Phe Thr Ser Arg Thr Pro Arg Asp Ser Pro Gly Ile Pro Pro
                355 360 365
Ser Ala Gly Ala His Gln Leu Phe Arg Gly Phe Ser Phe Val Ala Thr
        370 375 380
Gly Leu Met Glu Asp Asp Ser Lys Pro Arg Ala Thr Gln Ala Pro Leu
385 390 395 400
His Ser Val Val Gln Gln Leu His Gly Lys Asn Leu Val Phe Ser Asp
                                405 410 415
Gly Tyr Ile Val Lys Glu Thr Ile Gly Val Gly Ser Tyr Ser Val Cys
                        420 425 430
Lys Arg Cys Val His Lys Ala Thr Asn Met Glu Tyr Ala Val Ala Val
                435 440 445
Ile Asp Lys Ser Lys Arg Asp Pro Ser Glu Glu Ile Glu Ile Leu Leu
        450 455 460
Arg Tyr Gly Gln His Pro Asn Ile Ile Thr Leu Lys Asp Val Tyr Asp
465 470 475 480
Asp Ser Lys His Val Tyr Leu Val Thr Glu Leu Met Arg Gly Gly Glu
                                485 490 495
Leu Leu Asp Lys Ile Leu Arg Gln Lys Phe Phe Ser Glu Arg Glu Ala
                        500 505 510
Ser Phe Val Leu Tyr Thr Ile Ser Lys Thr Val Glu Tyr Leu His Ser
                515 520 525
Gln Gly Val Val His Arg Asp Leu Lys Pro Ser Asn Ile Leu Tyr Val
        530 535 540
Asp Glu Ser Gly Asn Pro Glu Cys Leu Arg Ile Cys Asp Phe Gly Phe
545 550 555 560
Ala Lys Gln Leu Arg Ala Glu Asn Gly Leu Leu Met Thr Pro Cys Tyr
                                565 570 575
Thr Ala Asn Phe Val Ala Pro Glu Val Leu Lys Arg Gln Gly Tyr Asp
                        580 585 590
Glu Gly Cys Asp Ile Trp Ser Leu Gly Val Leu Leu Tyr Thr Met Leu
                595 600 605
Ala Gly Tyr Thr Pro Phe Ala Asn Gly Pro Ser Asp Thr Pro Glu Glu
        610 615 620
Ile Leu Thr Arg Ile Ser Ser Gly Lys Phe Thr Leu Ser Gly Gly Asn
625 630 635 640
Trp Asn Thr Val Ser Glu Thr Ala Lys Asp Leu Val Ser Lys Met Leu
                                645 650 655
His Val Asp Pro His Gln Arg Leu Thr Ala Lys Gln Val Leu Gln His
                        660 665 670
Pro Trp Ile Thr Gln Lys Asp Lys Leu Pro Gln Ser Gln Leu Ser His
                675 680 685
Gln Asp Leu Gln Leu Val Lys Gly Gly Met Ala Ala Thr Tyr Ser Ala
        690 695 700
Leu Ser Ser Ser Lys Pro Thr Pro Gln Leu Lys Pro Ile Glu Ser Ser
705 710 715 720
Ile Leu Ala Gln Arg Arg Val Arg Lys Leu Pro Ser Thr Thr Leu
                                  725 730 735
```

### The alanine (“A”) residues substituted for lysine (“K”) residues in the native sequence to make the K94A/K447A RSK mutant of the present invention are shown in bold at positions 94 and 447 in SEQ ID NO: 2.

The K94A/K447A RSK mutation makes the RSK protein a “dominant negative” RSK mutant (DN-RSK). A dominant negative mutation creates a gene product (protein or polypeptide) that adversely affects the normal, wild-type gene product within the same cell, usually by dimerizing with the wild-type protein or polypeptide. The mutant p90RSK of the present invention may be made from any mammal including, but not limited to, rat, mouse, and human (including but not limited to Genbank Accession Nos. M99169, Swiss-Pro P16853, and Genbank Accession No.AF09042, which are hereby incorporated by reference in their entirety.)

Additional RSK mutants of the present invention include those known in the art or which may be characterized by amino acid insertions, deletions, substitutions, and modifications at one or more sites in or at the other residues of the native RSK polypeptide chain. (Spring et al., “Deletion of 11 Amino Acids in p90(rsk-mo-1) Abolishes Kinase Activity,” Mol Cell Biol 19(1):317-20 (1999); Roux et al., “Phosphorylation of p90 Ribosomal S6 Kinase (RSK) Regulated Extracellular Signal-Regulated Kinase Docking and RSK Activity,” Mol Cell Biol 23(14):4796-804 (2003); which are hereby incorporated by reference in their entirety). In accordance with this invention any such insertions, deletions, substitutions, and modifications should result in an RSK mutant that is rendered kinase inactive for cellular substrates including serine 703 (S703) phosphorylation of NHE1. Preferably, additional RSK mutants made according to the present invention would also be dominant negative mutants of RSK or would mimic the functional effects of an RSK mutant with regard to activation of p90RSK.

The RSK mutants of the present invention can be produced by any suitable method known in the art. Such methods include constructing a DNA sequence encoding the RSK mutants of the present invention and expressing those sequences in a suitably transformed host. This method will produce recombinant mutants of this invention. This technique is well known (Mourez et al., “Mapping Dominant-Negative Mutations of Anthrax Protective Antigen by Scanning Mutagenesis,” Proc. Natl. Acad. Sci. USA 100(24):13803-13808 (2003); Mark et al., “Site-specific Mutagenesis of The Human Fibroblast Interferon Gene,” Proc. Natl. Acad. Sci. USA 81:5662-66 (1984); U.S. Pat. No. 4,588,585, which are hereby incorporated by reference in their entirety).

Chemical synthesis can also be used to construct a DNA sequence encoding the RSK mutants of the present invention. For example, a nucleic acid molecule which encodes the desired RSK mutant may be synthesized by chemical means using an oligonucleotide synthesizer. Such oligonucleotides are designed based on the amino acid sequence of the desired RSK mutant, and preferably selecting those codons that are favored in the host cell in which the recombinant mutant will be produced. In this regard, it is well recognized that the genetic code is degenerate, i.e., that an amino acid may be coded for by more than one codon. Accordingly, it will be appreciated by one skilled in the art that for a given DNA sequence encoding a particular RSK mutant, there will be many degenerate DNA sequences that will code for that mutant. These degenerate DNA sequences are considered within the scope of this invention. Therefore, the present invention also encompasses suitable RSK mutants and degenerate variants thereof, which, in the context of this invention means all DNA sequences that code for a particular mutant.

Additional standard methods may be applied to synthesize a nucleic acid molecule encoding an RSK mutant of the present invention. For example, the complete amino acid sequence may be used to construct a back-translated gene. A DNA oligomer containing a nucleotide sequence coding for RSK mutant may be synthesized. For example, several small oligonucleotides coding for portions of the desired polypeptide may be synthesized and then ligated. The individual oligonucleotides typically contain 5′ or 3′ overhangs for complementary assembly.

The mutants of this invention may also be produced by a combination of chemical synthesis and recombinant DNA technology.

As used herein, comparison of the mutant p90RSK proteins can be made to wild type proteins. The wild type proteins can be naturally occurring variants of p90RSK as well as modified p90RSK proteins or polypeptides that possess substantially the same activity as the human or rat p90RSK of GenBank Accession Nos. AF090421 and M99169; which are hereby incorporated by reference in its entirety. By substantially the same, it is intended that the modified protein have at least 75%, preferably 85-115%, more preferably 95-100% of normal activity. The nucleic acid sequence encoding a RSK mutant of the present invention, whether prepared by site-directed mutagenesis, chemical synthesis, or other methods, may or may not also include DNA sequences that encode a signal sequence. Such signal sequence, if present, should be one recognized by the cell chosen for expression of the RSK mutant. It may be prokaryotic, eukaryotic or a combination of the two. It may also be the signal sequence of native RSK. The inclusion of a signal sequence depends on whether it is desired to secrete the RSK mutant from the recombinant cells in which it is made. If the chosen cells are prokaryotic, it generally is preferred that the DNA sequence not encode a signal sequence but include an N-terminal methionine to direct expression. If the chosen cells are eukaryotic, it generally is preferred that a signal sequence be encoded and most preferably that the wild-type RSK mutant signal sequence be used.

Once assembled (by synthesis, site-directed mutagenesis or another method), the nucleic acid sequences encoding an RSK mutant of this invention will be inserted into an expression vector and operatively linked to an expression control sequence appropriate for expression of the RSK mutant in the desired transformed host. Proper assembly may be confirmed by nucleotide sequencing, restriction mapping, and expression of a biologically active polypeptide in a suitable host. As is well known in the art, to obtain high expression levels of a transfected gene in a host, the gene must be operatively linked to transcriptional and translational expression control sequences that are functional in the chosen expression host.

The preparation of the nucleic acid constructs of the present invention including a nucleic acid molecule encoding a mutant RSK protein is carried out using methods well known in the art. U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture. Other vectors are also suitable.

Suitable vectors include, but are not limited to, vectors such as lambda vector system gt11, gt WES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK +/−or KS +/−(see “Stratagene Cloning Systems” Catalog (1993) from Stratagene, La Jolla, Calif., which is hereby incorporated by reference in its entirety), pQE, pIH821, pGEX, pET series (see F. W. Studier et. al., “Use of T7 RNA Polymerase to Direct Expression of Cloned Genes,” Gene Expression Technology Vol. 185 (1990), which is hereby incorporated by reference in its entirety), and any derivatives thereof. Several viral systems including murine retrovirus, adenovirus, parvovirus (adeno-associated virus), vaccinia virus, and herpes virus, such as herpes simplex virus and Epstein-Barr virus, and retroviruses, such as MoMLV have been developed as therapeutic gene transfer vectors (Nienhuis et al., Hematology, Vol. 16: Viruses and Bone Marrow, N. S. Young (ed.), pp. 353-414 (1993), which is hereby incorporated by reference in its entirety). Viral vectors provide a more efficient means of transferring genes into cells as compared to other techniques such as calcium phosphate or DEAE-dextran-mediated transfection, electroporation, or microinjection. It is believed that the efficiency of viral transfer is due to the fact that the transfer of DNA is a receptor-mediated process (i.e., the virus binds to a specific receptor protein on the surface of the cell to be infected.) Among the viral vectors that have been cited frequently for use in preparing transgenic mammal cells are adenoviruses (U.S. Pat. No. 6,203,975 to Wilson). In one embodiment of the present invention, the nucleic acid encoding the desired mutant RSK protein of the present invention is incorporated into an adenovirus expression vector.

Once a suitable expression vector is selected, the desired nucleic acid sequence(s) cloned into the vector using standard cloning procedures in the art, as described by Sambrook et al., Molecular Cloning: A Laboratory Manual, Cold Springs Laboratory, Cold Springs Harbor, N.Y. (1989), or U.S. Pat. No. 4,237,224 to Cohen and Boyer, which are hereby incorporated by reference in their entirety. The vector is then introduced to a suitable host. Thus, another aspect of the present invention is a p90RSK mutant nucleic acid molecule incorporated into an expression vector and a host. In a preferred embodiment this mutant is the K94A/K447A mutant nucleic acid molecule described herein above.

A variety of host-vector systems may be utilized to express the recombinant protein or polypeptide inserted into a vector as described above. Primarily, the vector system must be compatible with the host used. Host-vector systems include, without limitation, the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used to carry out this and other aspects of the present invention.

Different genetic signals and processing events control many levels of gene expression (e.g., DNA transcription and messenger RNA (“mRNA”) translation). Transcription of DNA is dependent upon the presence of a promoter, which is a DNA sequence that directs the binding of RNA polymerase, and thereby promotes mRNA synthesis. The DNA sequences of eukaryotic promoters differ from those of prokaryotic promoters. Furthermore, eukaryotic promoters and accompanying genetic signals may not be recognized in, or may not function in, a prokaryotic system, and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression see Roberts and Lauer, Methods in Enzymology, 68:473 (1979), which is hereby incorporated by reference in its entirety.

Promoters vary in their “strength” (i.e., their ability to promote transcription). For the purposes of expressing a cloned gene, it is desirable to use strong promoters in order to obtain a high level of transcription and, hence, expression of the gene. Depending upon the host system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the PR and PL promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Common promoters suitable for directing expression in mammalian cells include, without limitation, SV40, MMTV, metallothionein-1, adenovirus E1a, CMV, immediate early, immunoglobulin heavy chain promoter and enhancer, and RSV-LTR. Preferred promoters are cardiac-specific promoters. Exemplary cardiac-specific promoters include, without limitation, the α-myosin heavy chain promoter.

When multiple nucleic acid molecules are inserted, the multiple nucleic acid molecules may all be placed under a single 5′ regulatory region and a single 3′ regulatory region, where the regulatory regions are of sufficient strength to transcribe and/or express the nucleic acid molecules as desired.

Specific initiation signals are also required for efficient gene transcription and translation in prokaryotic cells. These transcription and translation initiation signals may vary in “strength” as measured by the quantity of gene specific messenger RNA and protein synthesized, respectively. The nucleic acid expression vector, which contains a promoter, may also contain any combination of various “strong” transcription and/or translation initiation signals. For instance, efficient translation in E. coli requires a Shine-Dalgamo (“SD”) sequence about 7-9 bases 5′ to the initiation codon (ATG) to provide a ribosome binding site. Thus, any SD-ATG combination that can be utilized by host ribosomes may be employed. Such combinations include but are not limited to the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used. Depending on the vector system and host utilized, any number of suitable transcription and/or translation elements, including constitutive, inducible, and repressible promoters, as well as minimal 5′ promoter elements, enhancers or leader sequences may be used.

Typically, when a recombinant host is produced, an antibiotic or other compound useful for selective growth of the transgenic cells only is added as a supplement to the media. The compound to be used will be dictated by the selectable marker element present in the plasmid with which the host was transformed. Suitable genes are those which confer resistance to gentamycin, G418, hygromycin, streptomycin, spectinomycin, tetracycline, chloramphenicol, and the like. Similarly, “reporter genes,” which encode enzymes providing for production of an identifiable compound identifiable, or other markers which indicate relevant information regarding the outcome of gene delivery, are suitable. For example, various luminescent or phosphorescent reporter genes are also appropriate, such that the presence of the heterologous gene may be ascertained visually.

An example of a marker suitable for the present invention is the green fluorescent protein (GFP) gene. The isolated nucleic acid molecule encoding a green fluorescent protein can be deoxyribonucleic acid (DNA) or ribonucleic acid (RNA, including messenger RNA or mRNA), genomic or recombinant, biologically isolated or synthetic. The DNA molecule can be a cDNA molecule, which is a DNA copy of a messenger RNA (mRNA) encoding the GFP. In one embodiment, the GFP can be from Aequorea victoria (Prasher et al., “Primary Structure of the Aequorea Victoria Green-Fluorescent Protein,” Gene 111(2):229-233 (1992); U.S. Pat. No. 5,491,084 to Chalfie et al., which are hereby incorporated by reference in their entirety). A plasmid encoding the GFP of Aequorea victoria is available from the ATCC as Accession No. 75547. Mutated forms of GFP that emit more strongly than the native protein, as well as forms of GFP amenable to stable translation in higher vertebrates, are commercially available from Clontech Laboratories, Inc. (Palo Alto, Calif.) and can be used for the same purpose. The plasmid designated pTα1-GFPh (ATCC Accession No. 98299, which is hereby incorporated by reference in its entirety) includes a humanized form of GFP. Indeed, any nucleic acid molecule encoding a fluorescent form of GFP can be used in accordance with the subject invention. Standard techniques are then used to place the nucleic acid molecule encoding GFP under the control of the chosen cell specific promoter.

The selection marker employed will depend on the target species and/or host or packaging cell lines compatible with a chosen vector.

A nucleic acid molecule encoding the desired RSK-encoding nucleic acid molecule (wild type or mutant) of the present invention, a promoter molecule of choice, including, without limitation, enhancers, and leader sequences; a suitable 3′ regulatory region to allow transcription in the host, and any additional desired components, such as reporter or marker genes, are cloned into the vector of choice using standard cloning procedures in the art, such as described in Sambrook et al., Molecular Cloning: A Laboratory Manual, Cold Spring Laboratory, Cold Spring Harbor, N.Y. (1989); Ausubel et al., “Short Protocols in Molecular Biology,” New York:Wiley (1999), and U.S. Pat. No. 4,237,224 to Cohen and Boyer, which are hereby incorporated by reference in their entirety.

Once the isolated nucleic acid molecule encoding a suitable nucleic acid molecule has been cloned into an expression vector, it is ready to be incorporated into a host. Recombinant molecules can be introduced into cells, without limitation, via transformation (if the host is a prokaryote), transfection (if the host is a eukaryote), transduction (if the host is a virus), conjugation, mobilization, or electroporation, lipofection, protoplast fusion, mobilization, particle bombardment, or electroporation, using standard cloning procedures known in the art, as described by Sambrook et al., Molecular Cloning: A Laboratory Manual, Second Edition, Cold Springs Laboratory, Cold Springs Harbor, N.Y. (1989), which is hereby incorporated by reference in its entirety. Suitable hosts include, but are not limited to, bacteria, virus, yeast, and mammalian cells, including, without limitation, mouse, and used to prepare the transgenic non-human animal of the present invention.

Alternatively, the RSK mutant-encoding nucleic acid molecule of the present invention may be inserted into a host cell and used as for studying RSK phosphorylation/NHE1 activation in vitro. Accordingly, another aspect of the present invention relates to a method of making a recombinant cell. Basically, this method is carried out by transforming a host with a nucleic acid construct of the present invention under conditions effective to yield transcription of the nucleic acid molecule in the host. Preferably, a nucleic acid construct containing a suitable nucleic acid molecule of the present invention is stably inserted into the genome of the recombinant host as a result of the transformation. Suitable host cells for the for the RSK mutant of the present invention includes, without limitation, cardiac muscle cells, smooth muscle cells, skeletal muscle cells, neuronal cells, and other cell types where reactive oxygen species, ischemia/reperfusion, and oxidative stress contribute to tissue dysfunction, cell impairment, and cell death. The cells may be from any mammalian species, including human. Suitable hosts for expression or other uses are bacterial or yeast cells, and viruses, as described herein above.

Transient expression allows quantitative studies of gene expression since the population of cells is very high (on the order of 106). To deliver DNA inside mammalian cells, several methodologies have been proposed, among them electroporation (Neumann et al., “Gene Transfer into Mouse Lyoma Cells by Electroporation in High Electric Fields,” EMBO J. 1:841-45 (1982); Wong et al., “Electric Field Mediated Gene Transfer,” Biochem Biophys Res Commun 30:107(2):584-7 (1982); Potter et al., “Enhancer-Dependent Expression of Human Kappa Immunoglobulin Genes Introduced into Mouse pre-B Lymphocytes by Electroporation,” Proc. Natl. Acad. Sci. USA 81:7161-65 (1984), which are hereby incorporated by reference in their entirety) and polyethylene glycol (PEG) mediated DNA uptake, Sambrook et al., Molecular Cloning: A Laboratory Manual, Chap. 16, Second Edition, Cold Springs Laboratory, Cold Springs Harbor, N.Y. (1989), which is hereby incorporated by reference in its entirety). During electroporation, the DNA is introduced into the cell by means of a reversible change in the permeability of the cell membrane due to exposure to an electric field. PEG transformation introduces the DNA by changing the elasticity of the membranes. Unlike electroporation, PEG transformation does not require any special equipment and transformation efficiencies can be equally high. Another appropriate method of introducing the nucleic acid construct of the present invention into a host is fusion of nucleic acid-containing vectors with other entities, either minicells, cells, lysosomes, or other fusible lipid-surfaced bodies that contain the chimeric gene (Fraley, et al., Proc Natl Acad Sci USA 79:1859-63 (1982), which is hereby incorporated by reference in its entirety).

Stable transformants are preferable for the methods of the present invention, which can be achieved by using variations of the methods above as describe in Sambrook et al., Molecular Cloning: A Laboratory Manual, Chap. 16, Second Edition, Cold Springs Laboratory, Cold Springs Harbor, N.Y. (1989), Ausubel et al., “Short Protocols in Molecular Biology,” New York:Wiley (1999), and U.S. Pat. No. 4,237,224 to Cohen and Boyer, which are hereby incorporated by reference in their entirety, and other methods known to those in the art.

The present invention provides a second transgenic non-human animal for the investigation of I/R injury and therapeutics for the prevention and treatment of I/R injury. This second transgenic non-human animal includes a transgene that encodes for cardiac-specific overexpression of wild type p90RSK compared to a non-transgenic animal.

The WT-p90RSK transgenic animal (WT-p90RSK-Tg) of the present invention overexpresses a wild-type RSK protein as a result of the introduction of a wild-type RSK-encoding nucleic acid molecule operably linked to an α-MHC promoter region for cardiac-specific expression of the wild-type RSK. An exemplary p90RSK nucleic acid molecule for use in making a WT-p90RSK-Tg animal is wild-type rat S6 protein kinase (RSK-1) from rat (Accession No. M99169), having SEQ ID NO: 3 as follows.

| | |
|---|---|
| cggcgcggcg gacggcccag ccagagcgcg aggggctggg gggcgtgcgg gggtatcggt | 60 |
| gcagcagcaa ggaccccggg gcccagaggc ggcacagccc ggggccgccc ggaggagcgc | 120 |
| gggcggtccg gcggcggcgc gATGccgctc gcccagctca aggaaccctg gccgctcatg | 180 |
| gagctggtgc cgctggaccc ggagaatgga caggcttcag gggaagaagc tggacttcag | 240 |
| ccatccaagg atgagggcat cctcaaggag atctctatca cacaccacgt caaggcaggc | 300 |
| tctgagaagg ctgatccatc ccattttgag ctcctcaagg ttctgggcca aggatccttt | 360 |
| ggcaaagtct tcctggtacg caaggtcacc cggcctgaca atgggcactt gtatgccatg | 420 |
| aaagtattaa agaaggccac gctgaaagtg cgtgaccgtg ttcggaccaa gatggagaga | 480 |
| gacatcctag ctgacgtgaa ccaccccttc gtagtgaaac tgcactatgc cttccagacc | 540 |
| gagggcaagc tctatcttat tctggacttt ctgcgtggtg gagacctgtt cacacgactc | 600 |
| tcaaaggagg ttatgtttac agaggaggat gtgaagtttt acctggctga gctggcactg | 660 |
| ggcctggacc acctgcacag cttgggcatc atttacagag acctcaagcc tgagaatatc | 720 |
| cttttggatg aggagggcca catcaaactc actgactttg gcctgagcaa ggaggccatt | 780 |
| gaccacgaaa agaaggccta ttccttctgc gggaccgtgg agtacatggc gcccgaggtt | 840 |
| gtcaaccgcc agggccacac ccacagtgca gattggtggt cctatggggt gttgatgttt | 900 |
| gagatgctga cgggctccct gcccttccag gggaaggacc ggaaggagac catgaccttg | 960 |
| attttgaagg caaagctagg catgccccag tttctgagca cggaagccca gagcctcctg | 1020 |
| cgggccctgt tcaagaggaa tcctgccaac cggcttggct caggccccga tggggctgag | 1080 |
| gaaattaaga gacatatctt ctactctacc attgactgga ataagctcta ccgccgtgag | 1140 |
| atcaagccac ctttcaagcc cgctgtggcc cagccggatg acaccttcta ctttgatacc | 1200 |
| gagttcacgt cacgcacacc cagggattcg ccgggcatcc cccccagtgc tggtgcccat | 1260 |
| cagctcttcc gtggcttcag cttcgtggcc accggtctga tggaggatga cagcaagcct | 1320 |
| cgggccaccc aggctccgct gcactcggtg gtacagcaac tccacgggaa gaacttggtt | 1380 |
| ttcagcgatg gctacatagt aaaggagacg atcggcgtgg gctcctactc tgtgtgtaag | 1440 |
| cgctgtgtcc acaaggccac caacatggag tacgcagtca aagtaatcga caaaagcaaa | 1500 |
| agagatccct ccgaagagat cgagattctt ctgcggtatg gacagcaccc caacatcatc | 1560 |
| accctgaaag atgtgtatga cgacagtaag cacgtatacc tggtgacaga gctgatgagg | 1620 |
| ggcggggagc tgctggataa gatcctacgg cagaaattct tctcagagcg ggaggccagc | 1680 |
| ttcgtcctgt acaccatcag caagactgtg gaatacttgc actcccaagg ggtcgtccac | 1740 |
| agggacctca aacccagtaa catcctgtat gtggatgagt ctgggaaccc cgaatgccta | 1800 |
| cgaatatgcg actttggctt tgccaagcag ctacgggctg agaacgggct tctcatgaca | 1860 |
| ccttgctaca cagccaactt tgtggcacct gaggtgctga agcgtcaggg ctacgatgaa | 1920 |
| ggctgtgaca tatggagcct gggcgttctg ctgtacacga tgctggcagg atacactcca | 1980 |
| tttgccaatg ggcccagtga taccccagag gagatcctca cccggatcag cagtgggaag | 2040 |
| ttcaccctca gtgggggaaa ctggaacacg gtttcagaga cagccaagga cttagtatct | 2100 |
| aagatgctgc atgtggaccc ccaccagcgc ctcacagcca aacaggttct gcagcacccg | 2160 |
| tggatcaccc agaaagacaa gctcccccag agccagttgt cccaccaaga cctgcagctt | 2220 |
| gtgaaggggg gcatggcagc tacatattct gcactcagta gctccaaacc caccccccag | 2280 |
| ctcaagccaa tcgagtcgtc catcctggcc cagcggcggg tgaggaagct gccatccacc | 2340 |
| accctgtgaa cgacagtgcg agcaaactcc tctgaggcag agtccttcca gagggagcaa | 2400 |
| gcctgagtca cagaccaagt ggaatggagt cctaaaggaa gcaactagcc cagctcaccc | 2460 |
| gtgcgggtgt gaagtgcctt cctccccagg acgggctctt ctgggctcag gctccattgt | 2520 |
| gtgaaatcca ctcactgtac aaactatttt taagaaagga aaaagaaaaa atgacatcat | 2580 |
| ttaccatgga tttttttttt acaagatcca tttggctttt tggccattgc agtcccagga | 2640 |
| ggaacaccca gtcccatgtg tggccaagac tcccgtgata gctttgggac tccgcccctc | 2700 |
| tgttggtcaa ggagccatct gcacccgcct ccgagcacgt tcggcgttgc ctctcagagt | 2760 |
| tgtcgactgg ctcctcagca gaacttggtg tccccagcca tctctttttc cattctgttc | 2820 |
| tggggttctc gaaccacttt ctgctaagag cccgggactc caccctgtgc agctcttggc | 2880 |
| tcaggcacca gcatccacag cgccccatgc gcagttgggc ccctgcagtc agaacgggca | 2940 |
| gccccgtgga gaggagacgg agagcacttt ttgggagact tcctgttctg ccactggaca | 3000 |
| gagttcacag gagaccaggg aggtagtcca cgggggatga gggctttttc cctttcctcc | 3060 |
| tcagctggta actcagggtt catctgtcca aggcctttct aataaaccta cagtccagtc | 3120 |
| aaaaaaaaaa a | 3131 |

The start codon for complete cDNA sequence for rat RSK is shown capitalized at position 142-144 in SEQ ID NO. 3. The amino acid sequence of the protein encoded by this cDNA shown above at SEQ ID NO:1. This nucleic acid sequence is a rodent sequence and is suitable for making a WT-p90RSK-Tg animal, as describe in greater detail in Example 7, below. Also suitable for use this aspect of the present invention is wild-type RSK from other mammal, including, but not limited to, mouse and human.

All aspects of the making and use of the DN-RSK transgenic non-human animal of the present invention disclosed herein apply also to the making and using of the WT-p90RSK-Tg transgenic animal in this aspect of the present invention, including the making of a construct containing a nucleic acid molecule encoding for a wild-type RSK protein, preparation of suitable mammalian expression vector, host cells, and host animals, methods of making and identifying WT-RSK transgenic non-human animals, and methods of using the WT-p90RSK-Tg animal as a model of I/R injury for identification of and assaying for therapeutic agents for prevention and treatment of I/R injury, such as that resulting from ischemia in an individual.

As describe in greater detail in the examples below, the cardiac overexpression of wild type p90RSK in this transgenic animal (WT-p90RSK-Tg, herein) has been characterized as exhibiting a novel mechanism of the renin-angiotensin system (RAS), as evidenced by the upregulation of pro-renin converting enzyme (PRECE) in WT-p90RSK-Tg. Thus, WT-p90RSK-Tg is highly suitable as an animal model of hyperrenin condition in mammals. Moreover, renin secretion and pro-renin processing are known to have causal significance in the pathogenesis of several clinical disorders, including heart disease, diabetes mellitus, and hypertension (King et al., “Hydrogen and potassium Regulation of (pro)renin Processing and Secretion,” Am J Physiol Renal Physiol 267:F1-F2 (1994), which is hereby incorporated by reference in its entirety). This has direct implications to ischemic myocardium (as described in detail herein below) and thus, provides a new paradigm for the treatment of ischemic myocardium in diabetic patients.

Therefore, in one aspect of the present invention, the WT-p90RSK-Tg animal is suitable as an animal model for diabetic cardiomyopathy. This model is suitable for studying the mechanism of I/R injury in diabetic (and hyperglycemic) individuals, and for the identification of agents for the inhibition of I/R injury due ischemic events in the diabetic individual. In this aspect of the present invention an individual is meant to include all mammals, including humans. In one embodiment of this aspect of the present invention, the individual has a diabetic or diabetic-like condition.

The present invention also relates to a method of treating an individual to inhibit ischemia reperfusion injury associated with an ischemic event. This method involves administering to an individual an effective amount of an agent that inhibits p90 ribosomal S6 kinase (p90RSK)-induced activation of pro-renin converting enzyme (PRECE), thereby inhibiting ischemia reperfusion injury associated with an ischemic event.

The present invention also relates to a method of identifying an agent that modulates ischemic reperfusion injury resulting from an ischemic event in a transgenic non-human animal whose genome comprises a transgene encoding for cardiac-specific overexpression of wild type p90 ribosomal S6 kinase (p90RSK). This method involves exposing the transgenic non-human animal to conditions effective to produce an ischemic event in the transgenic non-human animal; administering to the transgenic non-human animal an agent to be tested; and determining whether the agent modulates the ischemic reperfusion (I/R) injury resulting from the ischemic event in the transgenic non-human animal.

### EXAMPLES

The following examples are provided to illustrate embodiments of the present invention but are by no means intended to limit its scope.

### Materials and Methods for Examples 1-7

### Surgical Procedures

Non-transgenic littermate control (NLC) mice lacking the DN-RSK gene were used as controls. DN-RSK-Tg and WT male mice at 10 to 14 weeks of age were used. Mice were anesthetized with 2% halothane and 40% oxygen, and maintained with 0.5% halothane and 40% oxygen during open chest surgery. Tracheotomy was performed to provide artificial ventilation (0.3 ml tidal volume, 120 breaths/min), and the left coronary artery (LAD) was ligated with 8-0 nylon surgical suture 2.0 mm distal from tip of the left auricle (Maekawa et al., “Improved Myocardial Ischemia/Reperfusion Injury in Mice Lacking Tumor Necrosis Factor-Alpha,” J Am Coll Cardiol 39:1229-1235 (2002), which is hereby incorporated by reference in its entirety).

### Measurements of Infarct Area and Area at Risk

After a 45-min ligation and reperfusion, the LAD was re-occluded at the same location point and Evans blue dye was perfused from the left ventricular (LV) cavity. The heart was removed and cut transversely into five sections, which were incubated in 1.0% 2,3,5-triphenyltetrazolium chloride (TTC; Sigma, St. Louis, Mo.) for 20 min at 37° C. The area at risk (AAR) and infarct size (IS) correspond to the area unstained with Evans blue dye and the area unstained with TTC solution, respectively. The AAR to LV ratio and IS to LV ratio of each slice were determined using NIH Image version 1.63.

### Protein Extraction from Heart Tissue

Mouse hearts were washed with 10 ml of cold PBS. Ischemic and non-ischemic areas were identified by Evans-blue staining and the isolated ischemic tissues were frozen in liquid nitrogen and homogenized with 0.5 mL of lysis buffer (10 mM Tris-HCl pH 7.4, 0.15 M NaCl, 0.05% Triton X-100, 0.05% NP-40) containing 2 mmol/L sodium orthovanadate, and protease inhibitor cocktail (Sigma, St Louis, Mo.). Protein concentration was determined with the Bradford protein assay (Bio-Rad, Hercules, Calif.). Protein (30 μg) was separated on SDS-polyacrylamide gels and transferred to nitrocellulose membranes.

### Western Blot Analysis

Phospho-p90RSK (Thr359/Ser363) and p90RSK (695-708 of mouse RSK), phospho-ERK1/2 (Thr202/Tyr204) and JNK antibodies were purchased from Cell Signaling Corp (Beverly, Mass.). Active-JNK(Thr183/Tyr185) antibody was purchased from Promega (Madison, Wis.). ERK1/2 and 14-3-3 β antibodies were purchased from Santa Cruz Biotechnology (Santa Cruz, Calif.). The NHE1 antibody was purchased from Chemicon (Temecula, Calif.).

### In Vitro Kinase Assay

Protein lysates from the ischemic area were used for the in vitro kinase assay. Total protein (1 mg) was immunoprecipitated with RSK antibody (Cell Signaling Corp, Beverly, Mass.), and incubated with reaction buffer (25 mM HEPES, 10 mM MgCl₂, 10 mM MnCl₂, 10 mM ATP), ³²P-λ-ATP and RSK peptide (Upstate, Chicago, Ill.). Samples were blotted on filter paper (3M, St. Paul, Minn.) and washed with 0.75% phosphoric acid 3 times. Radioactivity was measured by liquid scintillation.

### Preparation of Rat Neonatal Cardiomyocytes and Adenoviral Transfection

For adenovirus preparation, the DN-RSK construct was cloned into the AdEasy™-CMV system (QBIOGene, Carlsbad, Calif.) using SalI and HindIII restriction enzymes.

Primary cultures of cardiac myocytes were prepared from ventricles of 1 to 3-day-old neonatal Wistar rats (Akimoto et al., “Heparin and Heparin Sulfate Block Angiotensin II-Induced Hypertrophy in Cultured Neonatal Rat Cardiomyocytes. A possible Role of Intrinsic Heparin-Like Molecules in Regulation of Cardiomyocyte Hypertrophy,” Circulation 93:810-816 (1996), which is hereby incorporated by reference in its entirety). Briefly, cells were dissociated by collagenase II (Worthington Biochem, NJ) from the ventricles and plated at a density of 1×10⁵ cells/cm² on 25 mm collagen-coated coverslips in Dulbecco's modified Eagle's medium (DMEM) with 10% fetal bovine serum and 10% horse serum. After 6 hrs of plating the isolated cardiomyocytes, 10 μM cytosine arabinoside (Ara C) was added and the cells were cultured 24 hrs, after which the culture medium was changed to DMEM with 10 μM Ara C in 10% fetal bovine serum.

### Measurement of NHE1 Activity in Neonatal Rat Cardiac Myocytes

Isolated neonatal cardiomyocytes were cultured on 25 mm glass coverslips. The intracellular pH indicator BCECF-AM was incubated with DMEM without FBS for 30 min at 37° C. (Ozkan et al., “A Rapid Method for Measuring Intracellular pH Using BCECF-AM,” Biochem Biophys Acta 1572: 143-148 (2002), which is hereby incorporated by reference in its entirety). The glass-cover slips were mounted into a modified Sykes-Moore chamber (Bellco, Vineland, N.J.) with Tris buffered saline solution (130 mm NaCl, 5 mm KCl, 1 0.5 mm CaCl2, 1.0 mm MgCl2, 20 mm HEPES, pH 7.4) at room temperature. For acid loading, 20 mM NH4Cl was added before recording. After 2 to 3 min acid loading, cells were washed with Tris buffered saline solution. The recording chamber was placed on an inverted microscope (Nikon Diaphot) equipped with epifluorescence. The field of interest was reduced to the area of a single cardiomyocyte by the viewfinder placed between the microscope and the photon multiplier tube (PMT; R928, Hamamatsu, Japan). BCECF-AM was excited at 490 and 440 nm, and the emission fluorescence recorded at 500 nm. 100 μM (Sabri et al., “Hydrogen Peroxide Activates Mitogen-Activated Protein Kinases and Na⁺-H⁺ Exchange in Neonatal Rat Cardiac Myocytes,” Circ Res 82:1053-1062 (1998), which is hereby incorporated by reference in its entirety).

### Cell Death Detection In Vitro

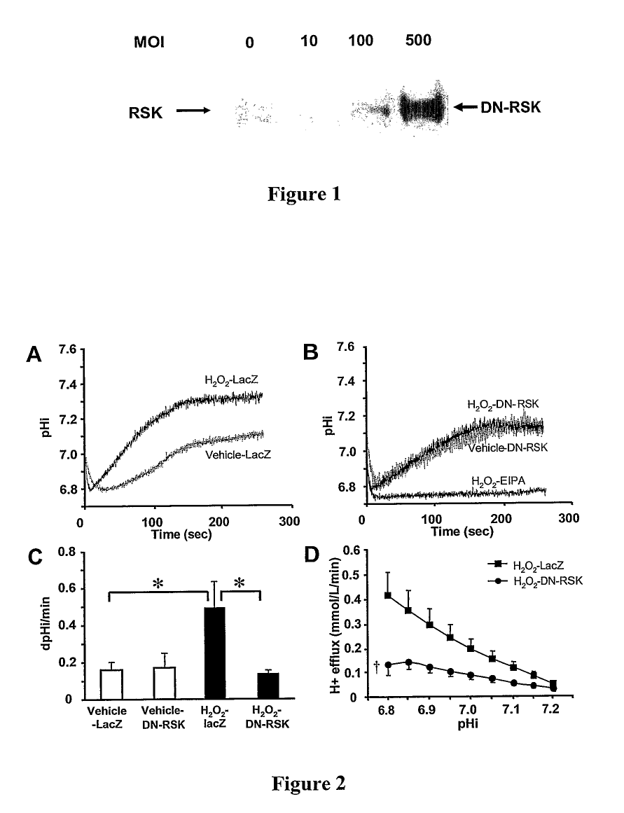

Ad.LacZ (LACZ gene in an adenoviral vector) and Ad.DN-RSK were transduced into neonatal rat cardiomyocytes at varying MOI, as shown in FIG. 1. There was a concentration-dependent expression of DN-RSK (Fire 1) with expression greater than endogenous RSK at 100 MOI. WT-RSK, WT-NHE1, and NHE S703A cDNAs were inserted into pLL3.7-IRES-EGFP to make a pLL3.7-WT-RSK-IRES-EGFP expression vector. These vectors were transfected into H9c2 rat embryonic myoblasts using lipofectamine 2000 (Invitrogen, Carlsbad, Calif.). Cells were cultured for 24 hr to allow sufficient protein expression, then cells were exposed to anoxia. Cells were placed for 12 hr in the anoxia chamber (5% CO2 and 95% N2) and after 24 hr, reoxygenation was performed by changing the medium and placing cells in an air incubator (5% CO2 and 95% air). After 24 hr cell death was detected by TUNEL and by cell death detection ELISA kit (Roche Applied Sciences, Indianapolis, Ind.). Only transfected cells identified by EGFP expression were counted to compare the effects of vector alone (pLL3.7-IRES-EGFP) vs. WT-RSK, WT-NHE1 and MHE1-S703A (pLL3.7-WT-RSK-IRES-EGFP, pLL3.7-WT-NHE1-IRES-EGFP and pLL3.7-NHE1-S703A-IRES-EGFP).

### Histopathology

NLC and DN-RSK-Tg hearts were removed and fixed by 4% formaldehyde. The fixed hearts were washed 3 times with 70% ethanol, embedded in paraffin, sectioned (5 μm thick), and stained by H&E (hematoxylin and eosin) or Masson trichrome stain. The fibrotic area was measured by NIH image version 1.63. LV area was calculated as the surface area of the LV at the widest section.

### Echocardiographic Analysis

Echocardiographic analysis with M-mode was performed in un-anesthetized mice using Acuson Sequoia C236 echocardiography machine equipped with a 15 MHz frequency probe (Siemens Medical Solutions, Malvern, Pa.). Left ventricular (LV) function was measured in the short axis view at midlevel, % fractional shortening (% FS) was assessed by measurement of the end-diastolic and end-systolic diameter (end-diastolic diameter-end-systolic diameter/end-diastolic diameter×100%).

### Example 1

### Generation of Cardiac Specific DN-RSK-Tg Mice

Rat RSK (SEQ ID NO: 1; GeneBank Acc. No: NM_{—}031107, which is hereby incorporated by reference in its entirety) was mutated to K94A/K447A to create a DN-RSK gene (SEQ ID NO: 2) encoding a kinase dead protein (Bjorbaek et al., “Divergent Functional Roles for p90rsk Kinase Domains,” J Biol Chem 270:18848-52 (1995), which is hereby incorporated by reference in its entirety) using the QuikChange site-directed mutagenesis kit (STRATAGENE, La Jolla, Calif.) (Dalby et al., “Identification of Regulatory Phosphorylation Sites in Mitogen-Activated Protein Kinase (MAPK)-Activated Protein Kinase-1a/p90rsk that are Inducible by MAPK,” J Biol Chem 273:1496-1505 (1998), which is hereby incorporated by reference in its entirety). The DN-RSK gene was cloned into a vector under the direction of the α-MHC (myosin heavy chain promoter region, Accession No. U71441) to allow for cardiac-specific (cardiomyocyte) expression (Gulick et al., “Isolation and Characterization of the Mouse Cardiac Myosin Heavy Chain Genes,” J Biol Chem 266:9180-9185 (1991), which is hereby incorporated by reference in its entirety). The α-MHC clone 26 was subcloned in the pBluescript II SK(+) vector by NotI site insertion. DNA was injected into fertilized mouse oocytes, derived from FVB mice, by the Transgenic Facility at the University of Rochester, and transgenic mice were produced form the transformed oocytes. Mice were maintained by breeding to FVB F1 animals (Jackson Laboratory, Bar Harbor, Me.).

An adenoviral DN-RSK construct (Ad.DN-RSK) was also produced by subcloning DN-p90RSK into a pShuttle-CMV vector SalI and Hind III sites, and recombinantly reproduced using methods well-known in the art.

PCR was used for identification of transgenic mice to detect the DN-RSK with A-MHC promoter constructs. Confirmation of the integration of the transgene was carried out using the following primer set:

forward:

(SEQ ID NO:4)

5′-ttagcaaacc tcaggcaccc ttaccccaca ta-3′,
and
reverse:

(SEQ ID NO:5)

5′-taggatgtct ctccatcttg gtccgaacac ggt-3′

to amplify the DN-RSK gene. All mice were used in accordance with guidelines of the National Institutes of Health for the care and use of laboratory animals.

### Example 2

### NHE1 Activity in Neonatal Rat Cardiomyocytes

To prove the essential role of RSK as a regulator of NHEL activity in the heart, neonatal rat cardiomyocytes were transduced with Ad.DN-RSK and Ad.LacZ (500 MOI), and NHE1 activity was measured, as shown in FIG. 1 and FIGS. 2A-D. In response to 100 μM H₂O₂, NHE1 activity increased 3-fold in LacZ expressing cardiomyocytes (0.16±0.02 to 0.49±0.13 pHi/min), as shown in FIG. 2A. In contrast, in cardiomyocytes expressing DN-RSK, H₂O₂ did not significantly stimulate NHE1 (0.17±0.08 to 0.14±0.03 pHi/min), as shown in FIG. 2B. The difference in rate of pHi recovery was highly significant (p<0.05), as shown in as shown in FIGS. 2C-D.

To show the difference in pHi recovery when NHE1 was inhibited by DN-RSK as compared to pharmacologic antagonism of transport, the potent NHE1 inhibitor EIPA was used, as shown in FIG. 2B. Pretreatment with 5 μM EIPA decreased pHi recovery to a much greater extent than DN-RSK (0.012±0.0001 pHi/min) significantly below acid stimulated recovery, as shown in FIG. 2A. Because NHE1 phosphorylation changes the affinity for H+, H+ efflux was also calculated. There was a dramatic decrease in H+ efflux in DN-RSK expressing cells over the pH range 6.8 to 7.2, suggesting a primary effect of DN-RSK on affinity NHE1 for H+, as shown in FIG. 2D. Western blotting for NHE1 showed no change in expression. These data show that DN-RSK prevents agonist-mediated activation of NHE1.

### Example 3

### Effect of DN-RSK and WT-RSK on Cardiomyocyte Cell Death

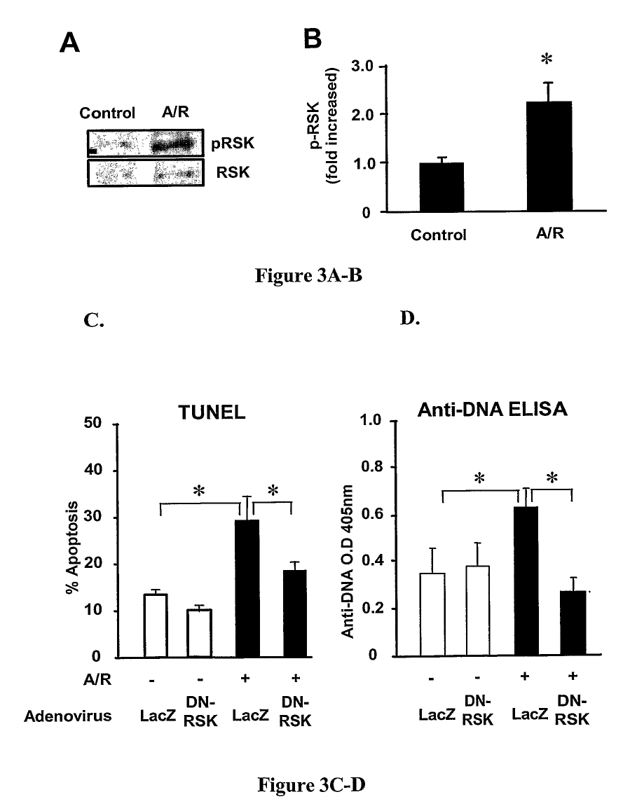

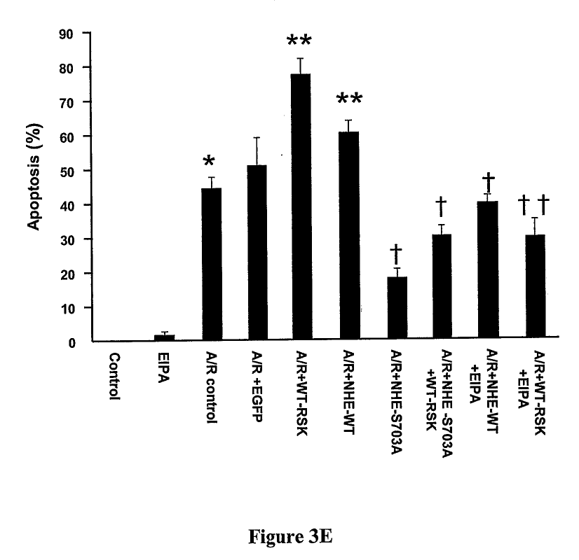

To provide further evidence for the importance of RSK-mediated activation of NHE1, the effect of altering RSK activity a study was carried out on cardiomyocyte apoptosis induced by anoxia for 12 hr followed by reoxygenation for varying times (A/R). Phosphorylation of endogenous RSK was significantly increased (2.3±0.4-fold, p<0.05) after A/R (12 hr/10 min), as shown in FIGS. 3A-B.

Next, the effect of overexpressing Ad.DN-RSK on rat neonatal cardiomyocyte death induced by A/R was studied. Cells were treated with A/R (12 br/24 hr). A/R significantly increased both TUNEL positive cells (10±2.8% to 32±3.1%, p<0.01) and DNA fragmentation (0.18±0.01 to 0.78±0.09, p<0.01), as shown in (FIGS. 3C-D). Transduction with Ad.LacZ or Ad.DN-RSK alone had no effect on apoptosis in the absence of A/R. However, DN-RSK transduced cardiomyocytes exhibited significantly decreased apoptosis compared to LacZ transduced cells (A/R Ad.LacZ; TUNEL 29.3±5.4%, ELISA 0.63±0.08 vs. A/R Ad.DN-RSK; TUNEL 18.6±2.0%, ELISA 0.27±0.06, p<0.05).

To provide further support that RSK-mediated phosphorylation of NHE1 S703 was responsible for the protective effect of DN-RSK, two additional experiments were performed: overexpression of WT-RSK and/or NHE1-S703A. Due to technical issues related to transfection efficiency, H9c2 cells were used. In H9c2 cells exposed to A/R, apoptosis was 44.4±3.4% and not significantly increased after transduction with pLL3.7-IRES-EGFP 51±8.1%, A/R+pLL), as shown in FIG. 3E. In contrast there was a significant increase in apoptosis in cells transduced with WT-RSK to 77.5±4.6% (A/R+WT-RSK, p<0.05). Transfection of NHE1-WT caused a small increase in apoptosis above that observed with A/R alone (A/R+NHE-WT, 61+4%). However, transfection of NHE1-S703A significantly decreased apoptosis compared to transfection with NHE1-WT. In fact, apoptosis of cells transfected with NHE1-S703A was significantly less than both controls (A/R control and A/R+EGFP). These data suggest that NHE1-S703A acts as a dominant negative for the signal events induced by A/R. A critical role for NHE1 activity in the pro-apoptotic effect of WT-RSK was shown by two findings. First, A/R-induced apoptosis was significantly reduced in H9c2 cells co-transfected with WT-RSK and NHE1-S703A. In these cells the increase in apoptosis stimulated by WT-RSK was significantly inhibited (to 30+5%, a 60% inhibition). Second, the increase in apoptosis stimulated by WT-RSK was significantly reduced in the presence of the NHEL inhibitor EIPA compared to untreated cells (EIP A+A/R+WT-RSK: 29.9±5.2%), as shown in FIG. 3E. The magnitude of inhibition by NHE1-S703A was similar to that observed with EIPA (39±4%, A/R+NHE-WT+EIPA). In summary, these data show that WT-RSK promotes H9c2 apoptosis induced by A/R, and the apoptosis is decreased by inhibiting NHE1 function pharmacologically (EIPA) or genetically (transduction of NHE1-S703A).

### Example 4

### Determination of I/R Infarct Area

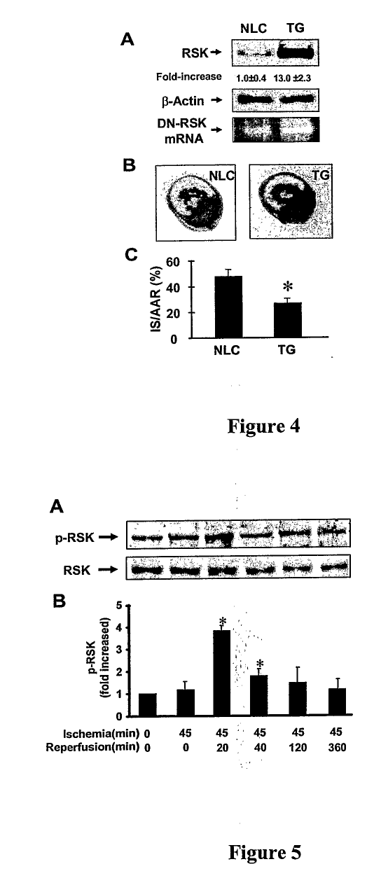

To determine the effect of inhibiting RSK on I/R injury in vivo, DN-RSK transgenic mice were generated (DN-RSK-Tg). Cardiac specific overexpression of DN-RSK in TG mice was confirmed by western blotting, as shown in FIG. 4A (top panel), and by PCR for the DN-RSK gene, shown in bottom panel of FIG. 4A. No difference in RSK expression was found in kidney.

In the DN-RSK-Tg mouse cardiac DN-RSK expression was 13 times higher than endogenous RSK in NLC heart. Under basal conditions, DN-RSK-Tg mice displayed no apparent cardiac phenotype compared to NLC mice (values similar to sham), as shown in Table 1, below. There were no significant differences between males and females. To assess the effect of DN-RSK on I/R injury, mice underwent 45 min of ischemia and 24 hr of reperfusion as describe in methods above. Infarct size, measured by TTC staining, was clearly greater in NLC than TG hearts, as shown in FIG. 4B. Quantitation of infarct-size (IS)/area-at-risk (AAR) is summarized in FIG. 4C, and shows that infarct size was significantly reduced in DN-RSK-Tg hearts compared with NLC hearts (NLC: 46.9±5.6% vs. DN-RSK-Tg: 26.0±4.2%, p<0.05, n=11). The AAR/LV did not differ significantly between NLC and DN-RSK-Tg mice (NLC: 62.5±2.9%, DN-RSK-Tg: 61.9±2.5%).

TABLE 1
Table 1. Histologic and echocardiographic analyses of LV dimensions
and function

| Sham | | I/R for 2 wks | |
|---|---|---|---|
| NLC | TG | NLC | TG |

Histopathology

| | | | | |
|---|---|---|---|---|
| IVSW (mm) | 1.7 ± 0.1 | 1.6 ± 0.1 | 1.6 ± 0.1 | 1.6 ± 0.1 |
| LVFW (mm) | 1.2 ± 0.1 | 1.2 ± 0.1 | 0.7 ± 0.1* | 0.9 ± 0.1† |
| LV area (mm²) | 2.3 ± 0.2 | 2.3 ± 0.1 | 5.8 ± 1.6* | 2.8 ± 0.4† |

Echocardiography and physiology

| | | | | |
|---|---|---|---|---|
| BW (g) | 28.7 ± 0.5 | 28.2 ± 0.7 | 30.8 ± 1.8 | 30.5 ± 1.5 |
| Heart Rate | 623 ± 12 | 639 ± 19 | 657 ± 14 | 644 ± 21 |
| (BPM) | | | | |
| HW (mg)/BW | 3.8 ± 0.1 | 3.8 ± 0.1 | 4.6 ± 0.2* | 4.1 ± 0.1† |
| (g) | | | | |
| LVDd (mm) | 2.6 ± 0.1 | 2.6 ± 0.1 | 3.5 ± 0.2* | 2.8 ± 0.1† |
| LVDs (mm) | 0.8 ± 0.1 | 0.8 ± 0.1 | 2.4 ± 0.2* | 1.3 ± 0.2† |
| % FS | 69.0 ± 2.0 | 69.0 ± 1.0 | 31.8 ± 4.6* | 52.8 ± 4.8† |

Values are group means ± S.E.;
n = 11 for each group.
LVDd, left ventricular dimension at diastolic;
LVDs, left ventricular dimension at systolic;
% FS, % fractional shortening;
IVSW, interventricular septal wall;
LVFW, left ventricular free wall.
LV area, left ventricular surface area measured in short axis at widest section.
*P < 0.05 vs. NLC sham group,
†P < 0.05 vs. NLC I/R group.

### Example 5

### Cardiac RSK Expression and RSK Phosphorylation

The effect of I/R on RSK phosphorylation as a measure of RSK activity was determined. The RSK phosphorylation peak at 20 min reperfusion is shown in blot, FIG. 5A. There was a low basal level of phosphorylation in the absence of I/R, as shown in FIG. 5B. After 45 min ischemia, p-RSK did not change, as shown in FIG. 5B, lane 2. However, after 45 min ischemia and 20 min reperfusion, endogenous p-RSK phosphorylation increased by 4-fold, as shown in FIG. 5B, lane 3. p-RSK returned to basal levels within 40 min of reperfusion, as shown in FIG. 5B. These data show that endogenous RSK is rapidly and transiently activated by I/R.

### Example 6

### NHEL Binding to 14-3-3 Increases After Cardiac I/R

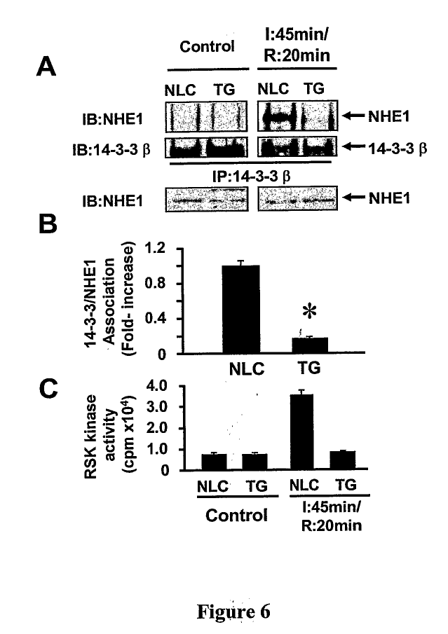

It was previously shown that RSK stimulated NHE1 activity by phosphorylating serine 703 (S703) and increasing binding of 14-3-3 (Takahashi et al., “p90RSK is a Serum-Stimulated NHE Kinase: Regulatory Phosphorylation of Serine 703 of Na⁺/H⁺ Exchanger Isoform-1,” J Biol Chem 274:20206-20214 (1999); Lehoux et al., “14-3-3 Binding to Na⁺/H⁺ Exchanger Isoform-1 is Associated With Serum-Dependent Activation of Na⁺/H⁺ Exchange,” J Biol Chem 276:15794-15800 (2001); Sabri et al., “Hydrogen Peroxide Activates Mitogen-Activated Protein Kinases and Na⁺-H⁺ Exchange in Neonatal Rat Cardiac Myocytes,” Circ Res 82:1053-1062 (1998), which are hereby incorporated by reference in their entirety). To relate NHE1 activity to RSK activity, binding of 14-3-3 to NHE1 was measured. Immunoprecipitation of 14-3-3 was performed followed by immunoblotting for NHE1 to assay their interaction, as shown in FIG. 6A. In mice subjected to sham procedure, binding of NHE1 to 14-3-3 was not detected in either TG or NLC heart tissue lysates. After I/R (45 min ischemia and 20 min reperfusion), 14-3-3 binding to NHE1 increased by 6.5±0.6-fold in NLC mice, compared to DN-RSK-Tg, as shown in FIG. 6A, upper panel. In contrast, there was markedly reduced 14-3-3 binding to NHE1 in DN-RSK-Tg hearts (p<0.05 vs. NLC). To prove that DN-RSK inhibited endogenous RSK activity after I/R, an in vitro kinase assay was performed. Hearts were exposed to I/R (45 min/20 min) and RSK was immunoprecipitated from lysates. Activity was measured by ³²P incorporation into a synthetic RSK substrate peptide. RSK kinase activity increased by ˜4 fold in NLC heart after I/R, but was completely inhibited in DN-RSK-Tg hearts, as shown in FIG. 6C). Therefore, as shown in FIGS. 6A, B, and C, DN-RSK prevents binding of 14-3-3 to NHE1 by inhibiting endogenous RSK in hearts exposed to I/R.

### Example 7

### Effect of DN-RSK on Functional Recovery 2 Weeks Post Reperfusion

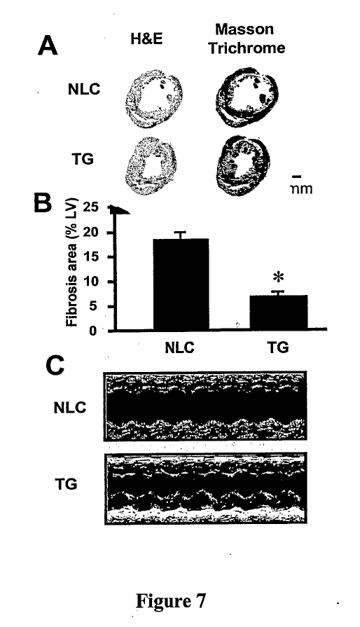

To determine the effects of DN-RSK on long-term LV functional recovery, mice were studied following 45 min ischemia and 2 weeks reperfusion (FIG. 7, Table 1, n=11). There were no significant differences in body weight (BW) or heart rate between DN-RSK-TG and NLC mice after sham operation or after 2 weeks of ischemia/reperfusion (Table 1). There was a significant (21%) increase in heart weight (HW) to BW in the NLC mice reflecting an enlarged LV in NLC mice. In contrast, there was a much smaller increase (8%) in HW/BW in the DN-RSK-Tg mice that was statistically less than in NLC mice (Table 1). Morphologic measures of ischemic damage were also significantly less in TG mice with increased LV free wall thickness (LVFW) and decreased LV area (a measure of LV dilation). Histologic analysis (Masson trichome stain) showed that DN-RSK-Tg hearts exhibited markedly less fibrosis 2 weeks after reperfusion (FIG. 7A), with a reduction in fibrotic area from 18.2±1.7% in NLC hearts to 6.7±0.9%, in DN-RSK TG hearts (FIG. 7B).

Echocardiographic analysis showed that LVDd, LVDs and % FS, as shown in Table 1, did not differ between NLC and TG sham mice. However, LVDd and LVDs were significantly smaller in TG than NLC hearts consistent with the histologic measurements (n=11, p<0.05). There was a highly significant improvement in % FS in TG hearts (n=11, p<0.05), consistent with improved systolic function in TG versus NLC.

### Discussion of Examples 1-7

As disclosed herein above, p90RSK is the primary regulator of NHE1 activity in cardiomyocytes exposed to I/R. Furthermore, cardiomyocyte specific expression of DN-RSK in a transgenic mouse decreases the extent of myocardial infarction and improves cardiac function after I/R. The mechanisms for the cardioprotective effect of DN-RSK are related to inhibiting NHE1 activity, as demonstrated by the several examples herein. First, decreased NHE1 activity was shown after I/R in DN-RSK expressing hearts, as measured by 14-3-3 binding. Second, it was shown that improved functional recovery two weeks after I/R occurred in DN-RSK expressing hearts compared to nontransgenic littermates. Third, increased apoptosis in H9c2 cells expressing WT-RSK was shown, which was inhibited by the NHE1 blocker, EIPA. Fourth, apoptosis was reduced in H9c2 cells that expressed NHE1-S703A, a mutant lacking the RSK phosphorylation site. These results are consistent with previous findings that 14-3-3 bound to NHE1 via phosphoserine 703 and increased NHE1 activity (Takahashi et al., “p90RSK is a Serum-Stimulated NHE Kinase: Regulatory Phosphorylation of Serine 703 of Na+/H+ Exchanger Isoform-1,” J Biol Chem 274:20206-20214 (1999); Lehoux et al., “14-3-3 Binding to Na⁺/H⁺ Exchanger Isoform-1 is Associated With Serum-Dependent Activation of Na⁺/H⁺ Exchange,” J Biol Chem 276:15794-15800 (2001), which are hereby incorporated by reference in their entirety).

Importantly, inhibition of NHE1 by blocking RSK decreases agonist-activated NHE1 function, without inhibiting basal, homeostatic NHE1 function. This result suggests that blocking RSK may be a better therapeutic strategy than NHE1 inhibitors (such as cariporide and zoniporide) that completely block ion transport as a mechanism to decrease sodium-hydrogen exchange and calcium overload during ischemia. While RSK has multiple cellular substrates, it appears that NHE1 is the critical substrate for the protective effect of DN-RSK based on three experiments. For example, it is shown herein above that WT-RSK overexpression in H9c2 cells stimulated apoptosis and that an NHE1 inhibitor could reverse the increase in apoptosis. Mechanistically, it was demonstrated that DN-RSK inhibits phosphorylation of S703 and binding of 14-3-3, an event previously shown to be required for activation of NHE1 Second, it was found that DN-RSK inhibited cardiomyocyte apoptosis induced by A/R in culture. Third, it was demonstrated that transduction of NHE1-S703A acted as a dominant negative for Na/H exchange and diminished apoptosis caused by A/R and by WT-RSK. A caveat is that it has not been shown that decreased phosphorylation of S703 is the only mechanism by which DN-RSK inhibits NHE-1 activity and apoptosis; thus, it is formally possible that alterations in other substrates and/or gene transcription may contribute to the protective effects.

NHE1 is regulated by multiple mechanisms in a tissue and stimulus specific manner. Four kinases have been identified that are putative NHE1 kinases: ERK1/2 (Bianchini et al., “The p42/p44 Mitogen-Activated Protein Kinase Cascade is Determinant in Mediating Activation of the Na+/H+ Exchanger (NHE1 isoform) in Response to Growth Factors,” J Biol Chem 272:271-279 (1997); Wang et al., “Phosphorylation and Regulation of the Na+/H+ Exchanger Through Mitogen-Activated Protein Kinase,” Biochemistry 36:9151-8 (1997), which are hereby incorporated by reference in their entirety); NIK (Yan et al. “The nck-Interacting Kinase (NIK) Phosphorylates the Na+-H+ Exchanger NHE1 and Regulates NHE1 Activation by Platelet-Derived Growth Factor,” J Biol Chem 276:31349-56 (2001), which is hereby incorporated by reference in its entirety); RSK (Takahashi et al., “p90RSK is a Serum-Stimulated NHE Kinase: Regulatory Phosphorylation of Serine 703 of Na+/H+ Exchanger Isoform-1,” J Biol Chem 274:20206-20214 (1999); Lehoux et al., “14-3-3 Binding to Na⁺/H⁺ Exchanger Isoform-1 is Associated With Serum-Dependent Activation of Na⁺/H⁺ Exchange,” J Biol Chem 276:15794-15800 (2001), which are hereby incorporated by reference in their entirety) and p160ROCK (Tominaga et al., “p160ROCK Mediates RhoA activation of Na—H Exchange,” Embo J. 17:4712-22 (1998), which is hereby incorporated by reference in its entirety). Several groups have characterized kinases activated in hearts exposed to I/R or cardiomyocytes exposed to H₂O₂ (Haworth et al., “Stimulation of the Plasma Membrane Na+/H+ Exchanger NHE1 by Sustained Intracellular Acidosis: Evidence for a Novel Mechanism Mediated by the ERK Pathway,” J Biol Chem 278:31676-31684 (2003); Moor et al., “Activation of Na+/H+ Exchanger-Directed Protein Kinases in the Ischemic and Ischemic-Reperfused Rat Myocardium,” J Biol Chem 276:16113-16122 (2001); Sabri et al., “Hydrogen Peroxide Activates Mitogen-Activated Protein Kinases and Na+-H+ Exchange in Neonatal Rat Cardiac Myocytes,” Circ Res 82:1053-1062 (1998), Wei et al., “Differential MAP Kinase Activation and Na+/H+ Exchanger Phosphorylation by H₂O₂ in Rat Cardiac Myocytes,” Am J Physiol Cell Physiol 281:C1542-1550 (2001), which are hereby incorporated by reference in their entirety). All groups found that both ERK1/2 and RSK were activated under these conditions. It was concluded that the upstream signaling pathway involved MEK1/2 since pretreatment of neonatal rat cardiomyocytes with two structurally distinct inhibitors, (PD98059 or U0126) inhibited activation of ERK1/2 and RSK and abolished stimulation of NHE activity by I/R or H₂O₂ (Sabri et al., “Hydrogen Peroxide Activates Mitogen-Activated Protein Kinases and Na+-H+ Exchange in Neonatal Rat Cardiac Myocytes,” Circ Res 82:1053-1062 (1998); Haworth et al., “Stimulation of the Plasma Membrane Na+/H+ Exchanger NHE1 by Sustained Intracellular Acidosis: Evidence for a Novel Mechanism Mediated by the ERK Pathway,” J Biol Chem 278:31676-31684 (2003); Moor et al., “Activation of Na+/H+ Exchanger-Directed Protein Kinases in the Ischemic and Ischemic-Reperfused Rat Myocardium,” J Biol Chem 276:16113-16122 (2001), which are hereby incorporated by reference in their entirety). Importantly, Rothstein et al. (“H₂O₂-Induced Ca2+ Overload in NRVM Involves ERK1/2 MAP Kinases: Role for an NHE-1-Dependent Pathway,” Am J Physiol Heart Circ Physiol 283:H598-605 (2002), which is hereby incorporated by reference in its entirety) suggested that H₂O₂ induced calcium overload was partially mediated by NHE-1 activation secondary to phosphorylation of NHE1. The present invention is the first to show that RSK activity is specifically required for NHE1 activation in cardiomyocytes in response to I/R and H₂O₂.

RSK consists of three isoforms (RSK1, RSK2, and RSK3) that show the same overall structure consisting of two kinase domains, a linker region and short N-terminal and C-terminal tails. The N-terminal kinase belongs to the AGC group of kinases, which include PKA and PKC. The N-terminal kinase phosphorylates the known substrates of RSK (Leighton et al., “Comparison of the Specificities of p70 S6 Kinase and MAPKAP Kinase-1 Identifies a Relatively Specific Substrate for p70 S6 Kinase: The N-Terminal Kinase Domain of MAPKAP Kinase-1 is Essential for Peptide Phosphorylation,” FEBS Lett 375:289-293 (1995), which is hereby incorporated by reference in its entirety). The C-terminal kinase belongs to the calcium/calmodulin-dependent kinase (CaMK) group of kinases. The only known function of the C-terminal kinase is regulation of the activity of the N-terminal kinase. Blenis and colleagues showed that the individual RSK1 kinase domains were under separate regulatory control (Richards et al., “Ribosomal S6 Kinase 1 (RSK1) Activation Requires Signals Dependent On and Independent of the MAP Kinase ERK,” Curr Biol 12:810-820 (1999), which is hereby incorporated by reference in its entirety). ERK1/2 phosphorylates RSK within the C-terminal kinase domain, while phosphoinositide-dependent kinase 1 (PDK1) phosphorylates RSK1 within the N-terminal kinase domain. In addition, it was previously shown that 14-3-3 is a negative regulator of RSK and agonist-mediated RSK activation requires dissociation of 14-3-38. The individual roles of 14-3-3, PDK1 and ERK1/2 in regulating RSK activation by I/R remain unknown. However, the present invention clearly establishes RSK as the primary regulator of NHE1 activation by H₂O₂ and I/R based on both in vivo and in vitro results with DN-RSK transgenic mice and DN-RSK adenovirus. The finding that NHE1-S703A apparently functions as a dominant negative suggests that phosphorylation of S703 may be necessary to stabilize NHE1 in an active state, perhaps via recruitment of other proteins.

Inhibition of NHE1 has been proposed as a therapeutic strategy for cardioprotection since both pharmacologic and molecular approaches that inhibit NHE1 are associated with reduced I/R injury. For example, the NHE1 inhibitors cariporide and zoniporide reduced I/R injury and improved recovery of heart function after I/R (Miura et al., “Infarct Size Limitation by a New Na+-H+ Exchange Inhibitor, Hoe 642: Difference From Preconditioning in the Role of Protein Kinase C.,” J Am Coll Cardiol 29:693-701 (1997); Chakrabarti et al., “A Rapid Ischemia-Induced Apoptosis in Isolated Rat Hearts and Its Attenuation by the Sodium-Hydrogen Exchange Inhibitor HOE 642 (Cariporide),” J Mol Cell Cardiol 29:3169-3174 (1997), which are hereby incorporated by reference in their entirety). In NHE1 null mice, there was also reduced I/R injury and improved functional recovery (Wang et al., “Mice With a Null Mutation in the NHE1 Na+-H+ Exchanger are Resistant to Cardiac Ischemia-Reperfusion Injury,” Circ Res. 93:776-82 (2003), which is hereby incorporated by reference in its entirety). As described herein above, cardiac specific DN-RSK over-expression improved LV function two weeks after I/R, as assessed by LV systolic dimensions and fractional shortening. There was a significant decrease in HW/BW in the TG mice compared to NLC mice, as shown in Table 1, which reflects a decrease in LV cavity size. Future studies will be necessary to elucidate the molecular mechanisms for changes in LV function and remodeling. However, in clinical trials that used the NHE1 inhibitors cariporide and eniporide (GUARDIAN and ESCAMI) to assess whether there was a benefit in patients experiencing myocardial infarction, no significant reduction in mortality was observed (Theroux et al., “Inhibition of the Sodium-Hydrogen Exchanger With Cariporide to Prevent Myocardial Infarction in High-Risk Ischemic Situations. Main Results of the GUARDIAN trial. Guard During Ischemia Against Necrosis (GUARDIAN) Investigators,” Circulation 102:3032-8 (2000); RuppTecht et al., “Cardioprotective Effects of the Na(+)/H(+) Exchange Inhibitor Cariporide in Patients with Acute Anterior Myocardial Infarction Undergoing Direct PTCA,” Circulation 101:2902-8 (2000), which are hereby incorporated by reference in their entirety). In the subgroup of patients who underwent coronary artery bypass grafting there was a 25% improvement in LV function with cariporide, suggesting that timing of drug administration and/or nature of ischemia and reperfusion are critical determinants for clinical outcome. The failure of NHE1 inhibitors to improve outcome also may be related to the fact that these inhibitors block the homeostatic functions of NHE1, which may lead to intracellular acidosis and cell death. The data presented herein suggests that targeted inhibition of RSK and reduction of NHE1 activity in response to agonists such as H₂O₂ (with preservation of NHE1 homeostatic function) is a novel strategy to treat cardiac I/R injury.

### Materials and Methods for Examples 8-14

### Protein Extract from Heart Tissue

Mouse hearts were washed with 10 ml of cold PBS. Isolated mice heart tissues were frozen in liquid nitrogen and homogenized with 0.5 mL of lysis buffer (10 mM Tris-HCl pH 7.4, 0.15 M NaCl, 0.05% Triton X-100, 0.05% NP-40) containing 2 mmol/L sodium orthovanadate, and protease inhibitor cocktail (Sigma, St Louis, Mo.). Protein concentration was determined with the Bradford protein assay (Bio-Rad, Hercules, Calif.). Protein (30 μg) was separated on SDS-polyacrylamide gels and transferred to nitrocellulose membranes.

p90RSK In Vitro Kinase Assays

Heart powder was homogenized with 3 vol of lysis buffer and centrifuged at 14,000 g (4° C. for 30 min), and protein concentration were determined. p90RSK was immunoprecipitated through the incubation of 1000 μg protein for each sample with 3 μl of the rabbit polyclonal anti-p90RSK (Santa Cruz, Santa Cruz, Calif.) antibody for 3 hrs, the addition of 40 μl of a 1:1 slurry of protein A/Sepharose beads to the extract/antibody mixture, and then incubation for 1 hour at 4° C. This complex was washed, twice each, in cell lysis buffer described above, LiCl buffer (500 mM LiCl 100 mM Tris-HCl (pH 7.6), 0.1% Triton X-100, 1 mM DTT) and wash buffer (20 mM HEPES, pH 7.2, 2 mM EGTA, 100 μM Na₃VO₄, 10 mM MgCl₂, 1 mM DTT, 0.1% Triton X-100). After the final wash and pelleting, S6 kinase substrate peptide was used to determine p90RSK kinase activity, as previously described (Cavet et al., “14-3-3beta is a p90 Ribosomal S6 Kinase (RSK) Isoform 1-Binding Protein That Negatively Regulates RSK Kinase Activity,” J Biol Chem 278(20):18376-18383 (2003), which is hereby incorporated by reference in its entirety). The in vitro kinase assay was performed according to manufacture's protocol using a long S6 kinase substrate peptide (Upstate) to determine radiolabeled phosphate incorporation by scintillation counter. Briefly, washed beads were incubated in 40 μl of Assay dilution buffer (20 mM MOPS, pH 7.2, 25 mM β-glycerol phosphate, 5 mM EGTA, 1 mM sodium orthovanadate, and 1 mM dithiothreitol), 10 μl of 150 μM of long S6 kinase substrate peptide, 100 μCi of (γ-³²P)ATP (Amersham Bioscience, Piscataway, N.J.), 100 μM of ATP, and 15 mM MgCl for 30 min at 30° C. The reaction was terminated by spotting 40 μl of reaction onto P81 phosphocellulose filter paper. The filter was washed five times in 0.75% phosphoric acid and one time in acetone for 5 min, radioactive incorporation was assayed by Cerenkov (liquid scintillation) counting.

### Measurement of Cardiac Damage

Creatine kinase (CK) and lactate dehydrogenase (LDH) were measured by the University of Rochester, Department of Clinical Chemistry, and reported in clinical indices (units/L) as means ±S.D.

### Western Blot Analysis

Heart powder was homogenized with 3 vol of lysis buffer and centrifuged at 14,000 g (4° C. for 30 min), and protein concentration was determined as previously described (Cameron et al., “Activation of Big MAP Kinase 1 (BMK1/ERK5) Inhibits Cardiac Injury After Myocardial Ischemia and Reperfusion,” FEBS Lett 566(1-3):255-260 (2004), which is hereby incorporated by reference in its entirety). Western blot analysis was performed as previously described (Yoshizumi et al., “Src and cas Mediate JNK Activation But Not ERK1/2 and p38 Kinases by Reactive Oxygen Species,” J Biol Chem 275(16): 11706-11712 (2000), which is hereby incorporated by reference in its entirety). In brief, the blots were incubated for 4 hr at room temperature with the anti-phospho-cardiac troponin I (Ser23/24) (Cell Signaling Technology, Inc., Beverly, Mass.), which recognizes dual phosphorylation of Ser 23 and Ser 24, anti-troponin I, anti-actin (Abcam, Cambridge, Mass.), anti-rat/mouse angiotensinogen (Research Diagnostics, Inc., Flanders, N.J.), Bcl-2 (Santa Cruz, Santa Cruz, Calif.) followed by incubation with horseradish peroxidase conjugated secondary antibody (Amersham, Piscataway, N.J.). Antibodies for assaying ERK1/2, p90RSK and PKCa/bII activation, anti-ERK1 or 2, p90RSK2, and PKCb antibody were from Santa Cruz (Santa Cruz, Calif.), and the phospho-ERK1/2 (Thr202/Tyr204), phospho-p90RSK (Thr359/Ser363), and phospho-PKCa/bII (Thr638/641) antibodies were from Cell Signaling (Cell Signaling Technology, Inc., Beverly, Mass.).

### Two-Dimensional Gel Electrophoresis (2-DE)

After hearts were perfused with PBS, ventricular tissue was immediately frozen in liquid nitrogen and ground to a fine powder using a liquid nitrogen-cooled mortar and pestle. The powder tissue were homogenized using a Polytron in solubilizing buffer composed of 7.5M urea, 1M thiourea, 4% CHAPS, 58 mM DTT, 0.2% biolyte pH 3-10, bromophenyl blue (trace), 10 μg/ml leupeptine, 10 μg/ml benzamidine, and 1 mM PMSF. The crude extract was then centrifuged at 14,000 g at 8° C. for 20 min. The supernatant was used immediately for 2-D analysis or stored at −80° C. for later use. First dimensional separation was performed by using the PROTEAN IEF cell apparatus (Biorad, Hercules, Calif.). Using the 7 cm focusing tray and readystripIPG (Bio-Rad) pH 4-7 we loaded 150 μg of protein per strip. All strips were re-hydrated overnight at room temperature in a re-swelling tray prior to isoelectric focusing. Isoelectric focusing (IEF) was performed from that point according to the manufacture's protocols, and IEF runs were stopped after 35,000 volt-hours. Upon completing of the electrofocusing, the IPG strips were equilibrated in an SDS buffer (6M Urea, 0.375M Tris pH 8.8, 2% SDS, 20% glycerol and 2.5% (w/v) iodoacetamide) for 30 min. After equilibration, the IPG strips were placed a top a 10% SDS-polyacrylamide slab gels and embedded with 0.5% agarose solution. Gels were run in the Protean 2 electrophoresis system (Bio-Rad, Hercules, Calif.) with running buffer (25 mM Tris, 192 mM glycine, 0.1% SDS) at 15° C. until the dye front reached the bottom of the gel. The completed 2-DE gels were stained with silver stained using the Bio-Rad silver staining kit according to Bio-Rad instruction.

### MALDI-TOF Mass Spectrometry Analysis

Tryptic digestion of pooled gel slices was subjected to enzymatic cleavage for the generation of peptide fragments. Pieces were washed with 100 mM ammonium bicarbonate, reduced (DTT) and alkylated (iodoacetamide), and then dehydrated via acetonitrile evaporation. The gel pieces were re-swollen with 25 mM ammonium bicarbonate containing ˜0.2 μg of enzyme to achieve a substrate/enzyme ratio of ˜10:1. ZipTip tippets (Millipore, Bedford, Mass.), packed with C18 matrix, were utilized to clean and concentrate peptide samples prior to analysis. Tips were washed with acetonitrile before peptides were bound and then eluted with either acetonitrile or matrix solution. ZipTip use affords a recovery of 50-70% in a 1 μl volume. Digested protein was mixed with the matrix a-cyano-4-hydroxycinnamic acid, and matrix-assisted laser desorption/ionization time-of-flight (MALDI-TOF) mass spectrometric analysis was performed as described previously (Florio et al., “Phosphorylation of the 61-kDa Calmodulin-Stimulated Cyclic Nucleotide Phosphodiesterase At Serine 120 Reduces Its Affinity for Calmodulin,” Biochemistry 33(30):8948-8954 (1994), which is hereby incorporated by reference in its entirety). Mass fingerprinting analysis and determination of phosphorylation was performed initially by MS-FIT (available from the UCSF website). The database search was considered significant if the protein was ranked as the best hit with a sequence coverage of more than 30%. Significance was defined as a MOWSE (Molecular Weight Search) score of at least 1e⁺⁰⁰³ (MS-FIT) or a difference in probability of 10⁻³ from the first to the second protein candidate (ProFound).

### Measurement of Left Ventricular Function by the Langendorff Preparation

For isolated heart from WT-p90RSK-Tg mice and non-transgenic littermate control (NLC) mice were studied using Langendorff preparation. Animals were anaesthetized with ketamine (50 mg/kg) and xylazine (2.5 mg/kg), i.p., and heparinized (5000 U/kg), i.p., to protect the heart against microthrombi. The chest was opened at the sternum and the heart, after cannulation with a 23 G phalanged stainless steel cannula, quickly removed. The heart was retrogradely perfused through the aorta in a non-circulating Langendorff apparatus with KH buffer (118 mM NaCl, 4.7 mM KCl, 1.2 mM MgSO₄, 1.2 mM KH₂PO₄, 2.5 mM CaCl₂, 25 mM NaHCO₃, 0.5 mM Na-EDTA and 11 mM glucose) at a constant pressure of 80 mmHg. The buffer was saturated with 95% O₂/5% CO₂ (v/v, pH 7.4, 37° C.) for 50 min. A homemade water-filled balloon was inserted into the left ventricle through the left atrium and was adjusted to a left ventricular end-diastolic pressure of 5 mmHg during initial equilibration. The distal end of the catheter was connected to an ETH-200 Bridge Amplifier (CB Sciences, Inc) and PowerLab/200 (AD Instruments) data acquisition system via a pressure transducer (DELTRAN II, Utah Medical Products, Inc., Midvale, Utah). Hearts were paced at 300 beats/min except during ischemia. Pacing was reinitiated after three minutes of reperfusion in all groups. After 25 minutes equilibration period with vehicle, captopril (50 μM, Sigma-Aldrich, St Louis, Mo.), or olmesartan (10 μM, Sankyo Pharma, Parsippany, N.J.), hearts were subjected to 20 or 40 min of no-flow normothermic global ischemia and 25 or 45 min reperfusion.

### Relative Quantitative RT-PCR

Total RNA isolation, first-strand cDNA synthesis, and relative quantitative reverse transcription-polyinerase chain reaction (RT-PCR) using Ambion's Competimer technology were performed as we described (Aizawa et al., “Role of Phosphodiesterase 3 in NO/cGMP-Mediated Anti-inflammatory Effects in Vascular Smooth Muscle Cells,” Circ Res 93(5):406-413 (2003), which is hereby incorporated by reference in its entirety). Ambion's competimer technology allows one to modulate the amplification of 18S rRNA in the same linear range as the RNAs under study when amplified under the same condition. The following primers were used for PCR analysis:

| | |
|---|---|
| PRECE: | |
| (SEQ ID NO: 6): | |
| 5′-atgtcgacca gtatgaggtt t-3′ | (sense) |
| and | |
| (SEQ ID NO: 7): | |
| 5′-tgactttctg taggtagact-3′ | (antisense); |
| BNP | |
| (SEQ ID NO: 8): | |
| 5′-ctgctggagc tgataagaga-3′ | (sense) |
| and | |
| (SEQ ID NO: 9): | |
| 5′-tgcccaaagc agcttgaga-3′ | (antisense); |
| ANF | |
| (SEQ ID NO: 10): | |
| 5′-gagaagatgc cggtagaaga-3′ | (sense), |
| and | |
| (SEQ ID NO: 11): | |
| 5′-aagcactgcc gtctctcaga-3′ | (antisense). |

### Analysis of Apoptosis

Cardiomyocyte apoptosis was measured by two different methodologies, the terminal deoxyribonucleotide transferase(TdT)-mediated dUTP nick-end labeling (TUNEL), and detecting in situ DNA fragmentation by anti-DNA fragmentation ELISA. TUNEL staining was performed using the In Situ Cell Death Detection Kit (Roche Diagnostics, Indianapolis, Ind.) as described previously (Ding et al., “Functional Role of Phosphodiesterase 3 in Cardiomyocyte Apoptosis Implication in Heart Failure,” Circulation 111 (19):2469-2476 (2005), which is hereby incorporated by reference in its entirety). For TUNEL method, cross sections of the heart were also stained for cardiomyocyte-specific sarcomeric α-actin with EA-53 to distinguish cardiomyocytes from contaminating fibroblasts and only EA-53 positive cells were counted. An average of total 1000 EA-53 positive cells from random fields were analyzed. All measurements were performed blinded.

### Statistical Analysis

Values presented are mean ±S.D. Statistical analysis was performed with the StatView 4.0 package (Abacus Concepts). Differences were analyzed with 1- or 2-way repeated measures ANOVA as appropriate, followed by Scheffe's correction.

### Example 8

### Preparation of Transgenic Mouse Line with Cardiac-Specific Overexpression of p90RSK

Rat wild type p90RSK1 cDNA was subcloned into a pBluescript-based Tg vector between the 5.5-kb murine-α-MHC promoter and 250-bp SV-40 polyadenylation sequences as previously described (Itoh et al., “Role of p90 Ribosomal S6 Kinase (p90RSK) in Reactive Oxygen Species and Protein Kinase C β (PKC β)-mediated Cardiac Troponin I Phosphorylation,” J Biol Chem 280(25):24135-24142 (2005), which is hereby incorporated by reference in its entirety). The purified transgene fragment was injected into male pronuclei of fertilized mouse oocytes (University of Rochester Transgenic Core). Genotype of mouse pups was confirmed by PCR analysis of tail clipping using standard procedure.

After a 6 hrs fast, basal blood samples were colleted from the tip of the tail. All blood samples were immediately measured for glucose using Prestige IQ, Blood Glucose Monitoring System (Home Diagnosis, Inc, Ft. Lauderdale, Fla.).

### Example 9

### p90RSK Activation in Streptozotocin-Induced Diabetic Mice

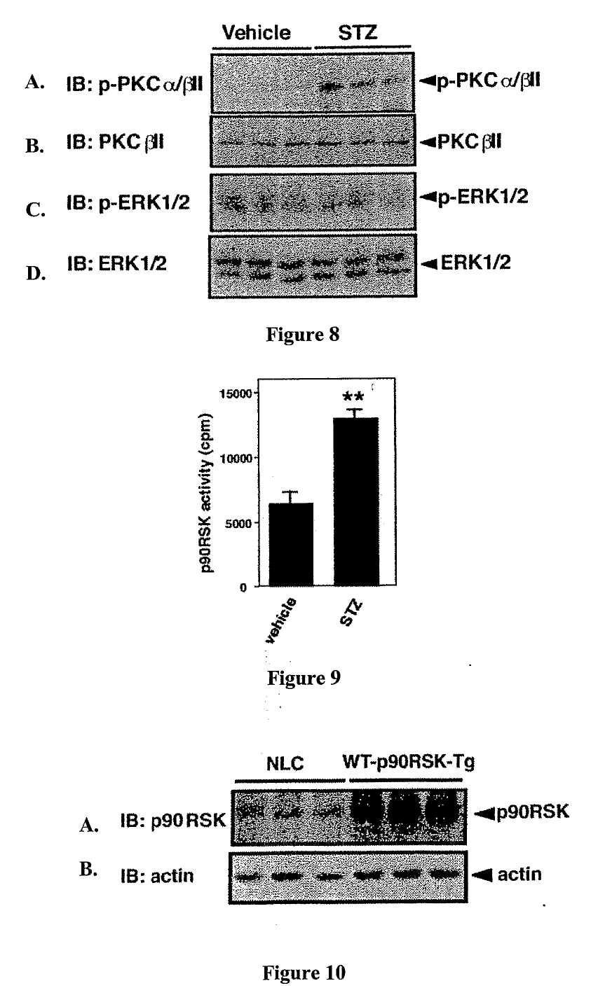

Previously, it was reported that PKCP activation is critical in H₂O₂-mediated p90RSK activation. In addition, it was found that p90RSK activity is significantly increased in cardiac specific PKCP overexpression mice (Itoh et al., “Role of p90 Ribosomal S6 Kinase (p90RSK) in Reactive Oxygen Species and Protein Kinase Cβ (PKC β)-mediated Cardiac Troponin I Phosphorylation, J Biol Chem 280(25):24135-24142 (2005), which is hereby incorporated by reference in its entirety). Since the critical role of PKCP activation in diabetes has been extensively studied, it was investigated whether p90RSK is activated in Streptozotocin (STZ)-induced hyperglycemic mice. The current study used STZ-induced diabetic mice, a known useful model for the study of diabetes (Aizawa-Abe et al., “Pathophysiological Role of Leptin in Obesity-related Hypertension,” J Clin Invest 105(9):1243-1252 (2000), which is hereby incorporated by reference in its entirety). STZ treatment significantly increased fasting blood glucose level after 2 weeks of injection (vehicle, 107±8 mg/dl vs. STZ, 224±5 mg/dl, p<0.01). As shown in FIGS. 8A-D, PKCα/βII, but not ERK1/2 phosphorylation, was significantly increased in STZ-induced hyperglycemic mice. p90RSK activation was also increased in hyperglycemic mice, as shown in FIG. 9, supporting the possible contribution of p90RSK in diabetic cardiomyopathy.

### Example 10

### Functional Role For p90RSK in I/R Injury in Cardiac-Specific WT-p90RSK-Tg Mice

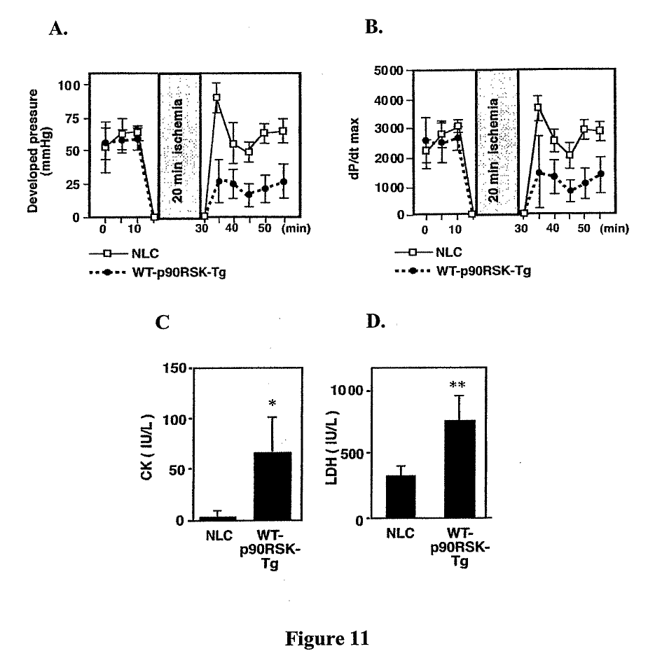

To examine the effect of p90RSK activation at the whole organ level, Tg mice with cardiac-specific expression of WT-p90RSK were made. The level of Tg protein expression in three different lines of Tg mice was determined by Western blot using an anti-p90RSK antibody. Because all three lines showed similar p90RSK expression level and phenotype, including the response to I/R in the Langendorff preparation, the data from line Tg-03 is described herein as the representative results for all WT-p90RSK-Tg mouse lines. The WT-p90RSK-Tg lines exhibited a 5 to 8-fold increase in total p90RSK expression relative to NLC mice, as shown in FIG. 11A-B. The WT-p90RSK-Tg lines exhibited normal feeding, activity, and weight gain up to 4 months of age compared to the NLC.

The basal phenotype and cardiac function of NLC and WT-p90RSK-Tg hearts were examined. Cardiac structure and function in 10-week old mice was normal as assessed by gross morphometric, histologic, and non-invasive echocardiographic measurements. A cross-section of both NLC and p90RSK-Tg hearts showed no change in ventricular wall thickness suggestive of cardiomyopathy and M-Mode echocardiographic images, as shown in Table 2 below and FIG. 7C, confirmed normal basal ventricular dimensions and function in live hearts until 4 months of age.

TABLE 2

| | 3 Months | | 10 Months | |
|---|---|---|---|---|
| | NLC | WT-p90RSK-Tg | NLC | WT-p90RSK-Tg |
| | n = 6 | n = 5 | n = 9 | n-5 |
| Heart Rate, bpm | 482.2 ± 23.3 | 463.8 ± 41.2 | 625.5 ± 11.3 | 634.5 ± 23.4 |
| LVEDd, mm | 2.6 ± 0.1 | 2.4 ± 0.1 | 2.8 ± 0.1 | 2.9 ± 0.1 |
| LVEDs, mm | 0.8 ± 0.4 | 0.8 ± 0.1 | .9 ± 0.1 | 1.3 ± 0.1^{a} |
| % FS | 69.3 ± 1.1 | 66.6 ± 2.0 | 67.1 ± 1.4 | 55.6 ± 2.9^{b} |
| IVSWd, mm | 1.0 ± 0.04 | 0.9 ± 0.03 | 0.91 ± 0.09 | 0.78 ± 0.04^{a} |
| PWd, mm | 1.0 ± 0.02 | 0.9 ± 0.02 | 0.96 ± 0.97 | 0.90 ± 0.06 |
| mVcf | 17.1 ± 0.4 | 17.5 ± 1.0 | 16.9 ± 0.4 | 13.6 ± 0.9^{b} |

ap < 0.05 versus NLC mice at 10 months of age.
bp < 0.01 versus NLC mice at 10 months of age.
bpm = heart beats per minute;
LVEDd = left ventricle end diastolic dimension;
LVESd = left ventricle end systolic dimension;
% FS = personal fractional shortening;
mVcf = mean velocity circumferential fiber shortening (mVcf).

The potential functional consequence of overexpression of WT-p90RSK in the Langendorff preparation was investigated. To exclude the cardiac from the circulatory effects, the isolated heart preparation was used to determine the “local” effect of p90RSK in cardiac function, especially after I/R. No difference in basal heart rate or contractile function was noted between NLC and WT-p90RSK-Tg hearts, and all hearts subjected to a 20 min period of global ischemia recovered their spontaneous heart beats. The recovery of left ventricular developed pressure after 20 min ischemia and reperfusion was over 90% of baseline for the NLC hearts. In contrast, the developed pressure only recovered to below 30% of baseline for the WT-p90RSK-Tg at all time points after ischemia and during reperfusion, as shown in FIG. 11A-D. A similar trend was seen in dP/dtmax with a significantly lower recovery of this parameter observed in WT-p90RSK-Tg hearts upon reperfusion, as shown in FIG. 11B The results strongly suggest that although WT-p90RSK-Tg hearts are functionally normal, they display significantly weaker contractile recovery compared to NLC after 20 min of ischemia.

To assess total cardiac damage incurred in the post-I/R heart, levels of creatine kinase (CK) and lactate dehydrogenase (LDH) released from the heart were measured. Perfusates collected from NLC hearts after 20 min of global ischemia and 25 min reperfusion documented no CK and modest LDH release, as shown in FIGS. 11C-D, respectively. WT-p90RSK-Tg mouse hearts subjected to the same insult demonstrated greater CK and LDH elevation, suggesting that p90RSK activation induced more severe FR damage.

### Example 11

### PRECE is Upregulated in WT-p90RSK-Tg Hearts

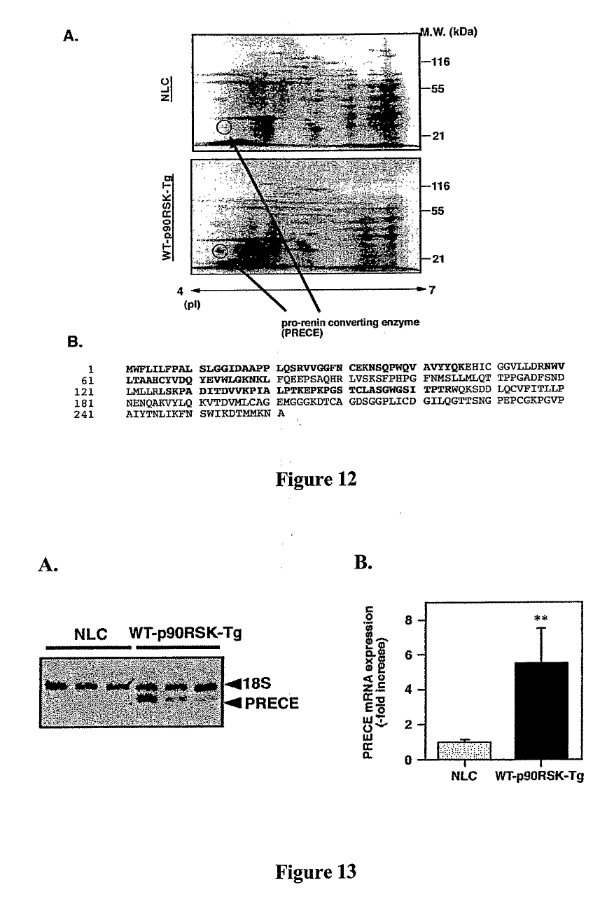

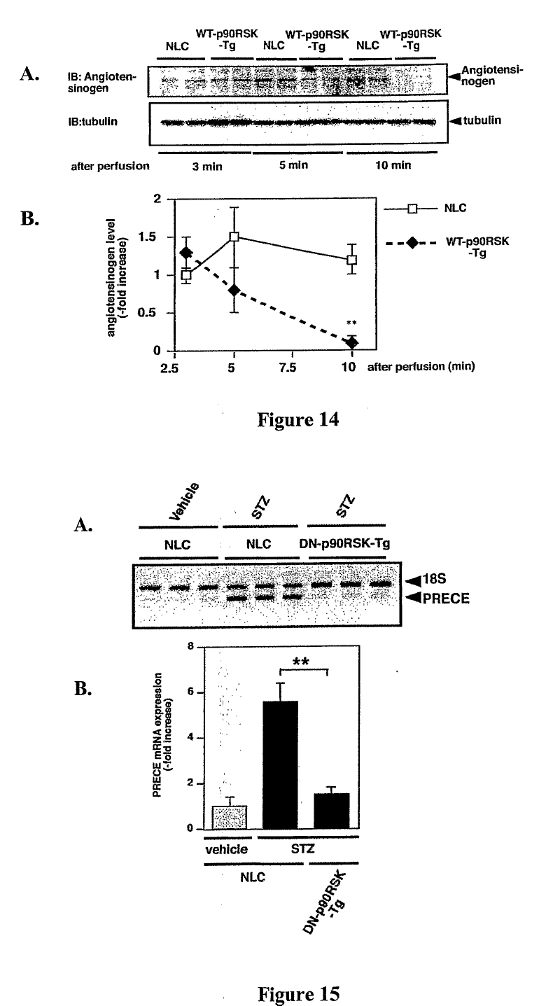

To characterize proteins that are specifically regulated by p90RSK activation, homogenates were prepared from NLC and WT-p90RSK-Tg hearts, and then analyzed by two-dimensional electrophoresis (2DE) and subsequent matrix-assisted laser desorption/ionization time-of-flight mass spectrometry (MALDI-TOF-MS) as described previously (Maekawa et al., “Inhibiting Ribosomal S6 Kinase (RSK) Prevents Na+/H+ Exchanger Isoform 1 (NHE1)-mediated Cardiac Ischemia-reperfusion (I/R) Injury,” Circulation (Abstract) 110(17):III-67 (2004), which is hereby incorporated by reference in its entirety). As shown in FIG. 12A, increased expression of a specific protein was detected by silver staining on two dimensional (2D) gel in p90RSK-Tg. Among the spots on 2D gel, the one most highly regulated was at 28 kDa, PI=6.4. This spot was identified as PRECE by MALDI-TOF mass spectrometric analysis with 100% fragment matching covering 40% of the total amino acid sequences of mouse PRECE (SEQ ID NO:12), shown in FIG. 12B. To confirm the enhanced PRECE expression in WT-p90RSK-Tg heart, reverse transcription-polymerase chain reaction (RT-PCR) was performed. As shown in FIG. 13A-B, the mRNA expression of PRECE was significantly increased in WT-p90RSK-Tg heart compared with NLC hearts. Since kallikrein-like PRECE can cleave not only pro-renin to renin, but also angiotensinogen to generate ang II directly (Urata et al., “Identification of a Highly Specific Chymase As the Major Angiotensin II-Forming Enzyme in the Human Heart,” J Biol Chem 265(36):22348-22357 (1990), which is hereby incorporated by reference in its entirety), the angiotensinogen protein level in NLC and WT-p90RSK-Tg mice was examined. As shown in FIGS. 14A-B, angiotensinogen levels in NLC mice declined slowly after KH buffer perfusion in the Langendorff model. In contrast, a significant rapid reduction of angiotensinogen content after perfusion was observed in WT-p90RSK-Tg mice. Taken together with the increase of PRECE expression in WT-p90RSK-Tg mice, these data suggest the increased angiotensinogen cleavage in WT-p90RSK-Tg mice, which is associated with increased ischemia/reperfusion damage.

### Example 12

### Involvement of p90RSK Activation on Hyperglycemia-Mediated PRECE Expression

Because p90RSK activation was increased in STZ-induced hyperglycemic mice, as shown in FIG. 8 and FIG. 9, it was determined whether PRECE expression is also increased in this diabetic model. PRECE mRNA expression was significantly increased in STZ-induced diabetic mice, as shown in FIGS. 15A-B. To determine the role of p90RSK activation in diabetes-mediated PRECE expression in heart, of cardiac specific DN-p90RSK-Tg mice were used. These mice showed no change in basal cardiac phenotype, but demonstrated cardio-protective effect against ischemia/reperfusion injury as previously described (Maekawa et al., “Inhibiting Ribosomal S6 Kinase (RSK) Prevents Na+/H+ Exchanger Isoform 1 (NHE1)-mediated Cardiac Ischemia-reperfusion (I/R) Injury,” Circulation (Abstract) 110(17):III-67 (2004), which is hereby incorporated by reference in its entirety). p90RSK activation was increased by STZ injection in NLC mice, but it was significantly inhibited in DN-p90RSK-Tg mice (FIG. 9 and NLC+STZ; 12991±1810 cpm, DN-p90RSK-Tg +STZ; 8009±797 cpm, mean ±S.D., p<0.05). As shown in FIGS. 15A-B, PRECE mRNA expression was increased by STZ injection in NLC, but not in DN-p90RSK-Tg mice, suggesting the critical role of p90RSK activation in STZ-induced PRECE expression in heart.

### Example 13

### Role of Renin Angiotensin System (RAS) in p90RSK-Mediated Enhancement of Cardiac Injury by I/R

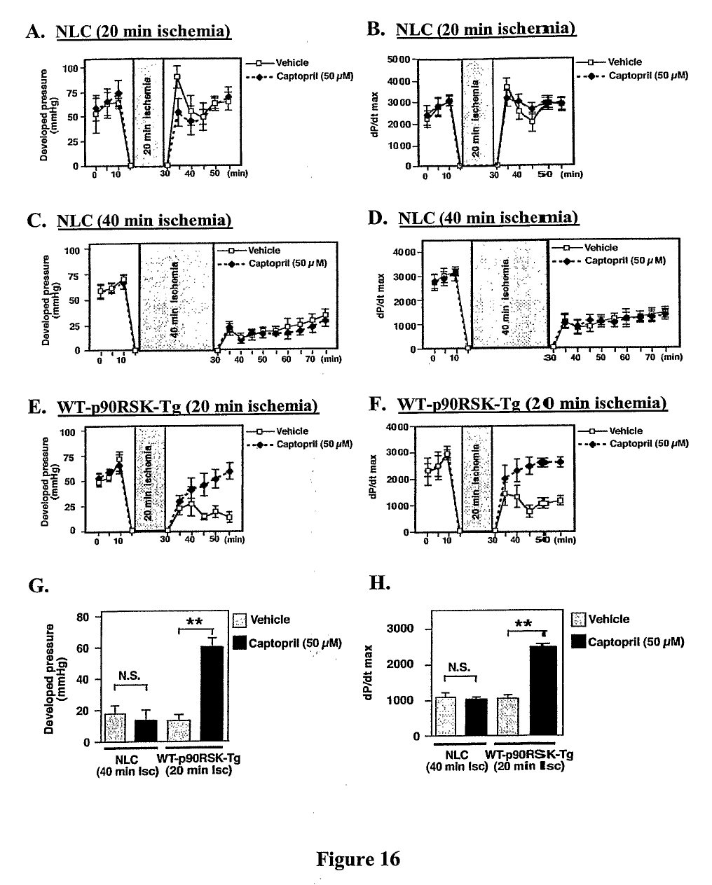

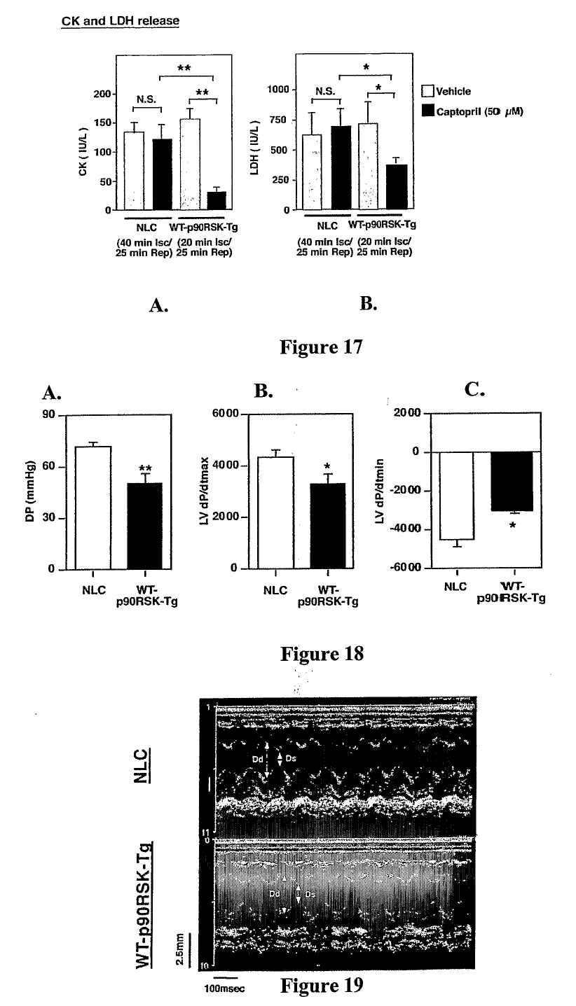

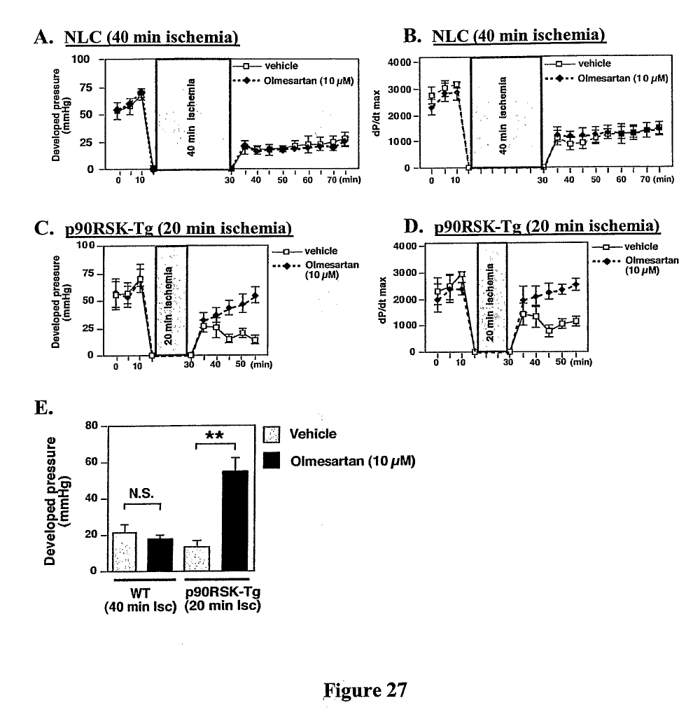

Because PRECE protein and mRNA expression were significantly increased in WT-p90RSK-Tg heart, it was investigated whether up-regulation of RAS by p90RSK-mediated PRECE could significantly enhance cardiac injury after I/R in WT-p90RSK-Tg. However, due to the rapid degradation of cardiac renin and ang II, along with residual contamination from serum, it is well recognized that accurate quantitation of these proteins is very difficult (Chapman et al., “Half-Life of Angiotensin II in the Conscious and Barbiturate-Anaesthetized Rat,” Br J Anaesth 52(4):389-393 (1980), which is hereby incorporated by reference in its entirety). Therefore, the contribution of RAS in p90RSK-mediated cardiac dysfunction was investigated by evaluating the effect of ACE inhibitors and angiotensin II type 1 (AT1) receptor blockers on recovery of cardiac function after I/R. Under the condition of 20 min ischemia, the developed pressure of NLC could almost completely recover, as shown in FIGS. 16A-B, but the recovery of developed-pressure after reperfusion in WT-p90RSK-Tg hearts was around 30% of the basal level as previously shown in FIG. 11, and FIG. 16E. As shown in FIGS. 16A-B and 16C-D, the pre-treatment with ACE inhibitor (captopril, 50 μM) had no effect on the recovery after I/R in NLC mice. Of note, since in NLC mice almost full recovery of cardiac function after 20 min ischemia was observed, prolonged 40 min ischemia in NLC hearts was also performed, as shown in FIGS. 16C-D. Forty min ischemia in NLC reduced cardiac function to around 30% of basal levels, and resulted in similar recovery to that of WT-p90RSK-Tg subjected to a shorter 20 min ischemic episode. However, no beneficial effect of ACE inhibitor was detected, even after 40 min ischemia in NLC hearts, as shown in FIGS. 16C-D and 16G-H, which is consistent with previous reports in rodents from several different laboratories (Liu et al., “Paracrine Systems in the Cardioprotective Effect of Angiotensin-converting Enzyme Inhibitors on Myocardial Ischemia/Reperfusion Injury in Rats,” Hypertension 27(1):7-13 (1996); Nakano et al., “Role of the Angiotensin II Type 1 Receptor in Preconditioning Against Infarction,” Coron Artery Dis 8(6):343-350 (1997); Harada et al., “Angiotensin II Type 1A Receptor Knockout Mice Display Less Left Ventricular Remodeling and Improved Survival After Myocardial Infarction,” Circulation 100(20):2093-2099 (1999), which are hereby incorporated by reference in their entirety). In contrast, the pre-treatment with ACE inhibitor in WT-p90RSK-Tg mice resulted in significant improvement in the recovery of cardiac function after 20 min of ischemia, as shown in FIGS. 16G-H. Similar protective effects were also found using an AT1 receptor blocker (olmesartan, 10 μM) in WT-p90RSK-Tg mice, as shown in FIGS. 27C-E. The level of cardiac enzymes, CK and LDH, released from the ischemic heart were measured, as shown in FIG. 17A-B. Perfusates collected from NLC mice hearts after 40 min of global ischemia and 25 min reperfusion documented elevated CK and LDH levels, but the pretreatment of captopril showed no beneficial effect on the release of CK and LDH in NLC hearts. Since after 40 min of global ischemia WT-p90RSK-Tg could not regain any contractile function, we selected a 20 min ischemic period in WT-p90RSK-Tg. In contrast to NLC, captopril significantly reduced release of these cardiac enzymes after 20 min ischemia and 25 min reperfusion in WT-p90RSK-Tg hearts, consistent with cardiac function data shown in FIG. 6. Since α-MHC promoter derived p90RSK expression is selectively induced in cardiomyocytes and our data is demonstrated in isolated heart preparations, these data suggest the enhancement of local cardiac RAS in WT-p90RSK-Tg. The activation of local cardiac RAS is consistent with the increase of PRECE expression in WT-p90RSK-Tg hearts.

### Example 14

### WT-p90RSK-Tg Show Cardiac Dysfunction After 8 Months of Age with Increasing Apoptosis and Interstitial Fibrosis

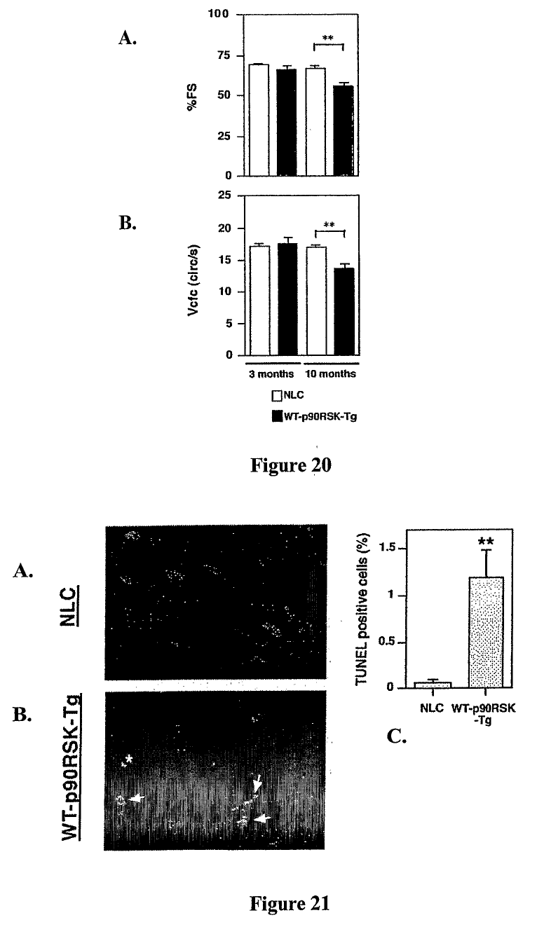

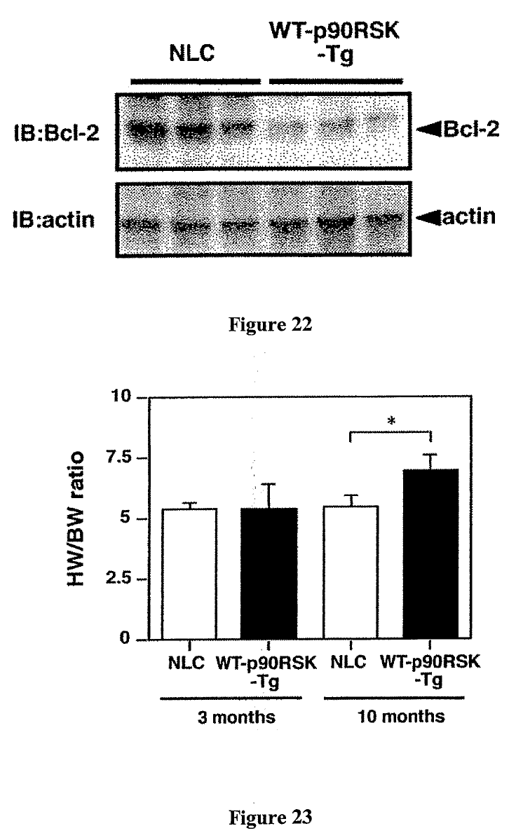

Although no significant pathological phenotype was observed in WT-p90RSK-Tg up to 4 months of age, it was found that WT-p90RSK-Tg mice displayed a significant impairment in cardiac contractility as assessed by decreased dP/dt and developed pressure (DP) at about 10 months of age, as shown in FIGS. 18A-C. Since no significant difference in heart rate was found (NLC; 480±23 bpm, WT-p90RSK-Tg; 455±21 bpm, mean ±S.D., p=n.s.), these differences are most likely not due to the depth of anesthesia. To confirm the functional invasive hemodynamic alterations, echo-cardiographic measurements were performed, which showed that both fractional shortening (FS) and velocity of circumferential fiber shortening (Vcfs) were reduced in WT-p90RSK-Tg mice at 8 to 10 months of age (Table 2, and FIGS. 19-20), again indicating impairment of contractile function. Since impairment of contractile function was observed in WT-p90RSK-Tg mice, apoptosis in WT-p90RSK-Tg mice was also examined. As shown in FIGS. 21A-B, there was a significant increase in apoptotic cells in WT-p90RSK-Tg mice compared with NLC by TUNEL assay. Bcl-2 is a well-known anti-apoptotic molecule and its expression can be repressed by angiotensin II (Ding et al., “Functional Role of Phosphodiesterase 3 in Cardiomyocyte Apoptosis: Implication in Heart Failure,” Circulation 111(19):2469-2476 (2005), which is hereby incorporated by reference in its entirety). Decreased Bcl-2 expression levels were observed in WT-p90RSK-Tg mice. These data also support that p90RSK activation promotes apoptosis probably via repression of Bcl-2 expression, as shown in FIG. 22.

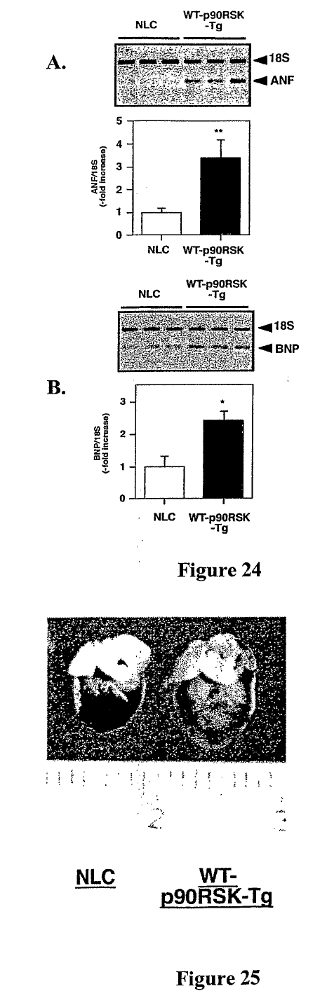

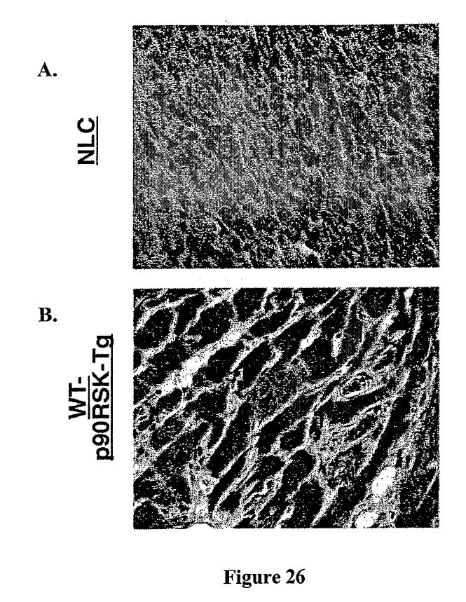

Normalized cardiac mass (HW/BW ratio) was slightly increased in WT-p90RSK-Tg mice at 8 to 10 months of age, but not at 3 months of age, as shown in FIG. 23. Expression of molecular markers of cardiac hypertrophy such as atrial natriuretic factor (ANF) and brain natriuretic protein (BNP) were also increased in WT-p90RSK-Tg compared with NLC at 8-10 months, as shown in FIGS. 24A-B. Slightly increased heart size in WT-p90RSK-Tg mice was observed at 10 months of age, as shown in FIG. 25. Histologically, an increase in overall heart size was observed characterized by interstitial fibrosis and hypertrophied cardiomyocytes in WT-p90RSK-Tg compared with NLC, as shown in FIGS. 26A-B. These data demonstrate an increase in interstitial fibrosis with apoptosis in WT-p90RSK-Tg mice at 10 months, which mimics diabetic cardiomyopathy as previously described (Bell DS, “Diabetic Cardiomyopathy. A Unique Entity or a Complication of Coronary Artery Disease?” Diabetes Care 18(5):708-714 (1995), which is hereby incorporated by reference in its entirety).

### Discussion of Examples 8-14

Meta-analysis of ACE inhibitor trials provide compelling evidence that ACE inhibitors attenuate the detrimental effects of ang II, improve survival, and reduce morbidity in patients with acute myocardial infarction and heart failure. However, the mechanism for the larger effects of ACE inhibitors in diabetic patients remains unclear. In the present study it was found that p90RSK activation was increased in diabetic hearts, and PRECE protein and mRNA levels were specifically up-regulated in WT-p90RSK-Tg hearts. Increased PRECE mRNA expression levels were detected in hearts of mice with STZ induced diabetes. This is believed to be the first report to document the possible role and expression of PRECE in heart. It was found that although ACE inhibitor did not improve recovery of cardiac function after I/R in NLC hearts, in contrast, there was significant improvement in the recovery of cardiac function and damage by both ACE inhibitor and AT1 receptor blocker in WT-p90RSK-Tg hearts. These data provide a novel mechanism of RAS in ischemic myocardium and a new paradigm for the treatment of ischemic myocardium in diabetic patients. Previous data have shown controversial results about the effect of AT1 blocker on cardiac damage after I/R among different species. In mouse, rat, and rabbit, no significant protective effect has been shown by AT1 blocker and in angiotensin II type 1A receptor knockout mice, especially within one week after ischemia/reperfusion (Liu et al., “Paracrine Systems in the Cardioprotective Effect of Angiotensin-converting Enzyme Inhibitors on Myocardial Ischemia/Reperfusion Injury in Rats,” Hypertension 27(1):7-13 (1996); Nakano et al., “Role of the Angiotensin II Type 1 Receptor in Preconditioning Against Infarction,” Coron Artery Dis 8(6):343-350 (1997); Harada et al., “Angiotensin II Type 1A Receptor Knockout Mice Display Less Left Ventricular Remodeling and Improved Survival After Myocardial Infarction,” Circulation 100(20):2093-2099 (1999), which are hereby incorporated by reference in their entirety). In contrast, in dog and swine models, AT1 receptor blocker could inhibit 40 to 50% of infarct size (Ford et al., “Intrinsic ANG II Type 1 Receptor Stimulation Contributes To Recovery of Postischemic Mechanical Function,” Am J Physiol 274(5 Pt 2):H1524-1531 (1998); Jalowy et al., “AT1 Receptor Blockade in Experimental Myocardial Ischemia/Reperfusion,” Basic Res Cardiol 93(Suppl 2):85-91 (1998), which are hereby incorporated by reference in their entirety). Therefore, it is intriguing to speculate that the previous controversial results regarding the effect of RAS inhibitors after I/R may be due to the different expression of PRECE among the different strains and species.

The existence of a local RAS in the heart is still a controversial issue. The supporting evidence for local RAS comes from the beneficial effect of the ACE inhibitors in heart failure, which are independent, at least partially, of their effect on blood pressure (Danser et al., “Prorenin, Renin, Angiotensinogen, and Angiotensin-converting Enzyme in Normal and Failing Human Hearts. Evidence for Renin Binding,” Circulation 96(1):220-226 (1997); Pfeffer et al., “Effect of Captopril On Mortality and Morbidity in Patients With Left Ventricular Dysfunction After Myocardial Infarction. Results of the Survival and Ventricular Enlargement Trial. The SAVE Investigators,” N Engl J Med 327(10):669-677 (1992), which are hereby incorporated by reference in their entirety). Based on the previous data, although all RAS components are present in cardiac tissue and both ang I and ang II are generated in the heart, the majority of ang I and ang II present in cardiac tissue sites originates from the circulation, and is therefore kidney-derived pro-renin and renin (Danser et al., “Prorenin, Renin, Angiotensinogen, and Angiotensin-converting Enzyme in Normal and Failing Human Hearts. Evidence for Renin Binding,” Circulation 96(1):220-226 (1997); Pfeffer et al., “Effect of Captopril On Mortality and Morbidity in Patients With Left Ventricular Dysfunction After Myocardial Infarction. Results of the Survival and Ventricular Enlargement Trial. The SAVE Investigators,” N Engl J Med 327(10):669-677 (1992), which are hereby incorporated by reference in their entirety). One of the mechanisms by which the heart may regulate its ang I and ang II concentrations independent of the circulating levels of these RAS components is the rate of conversion of pro-renin to active renin by proteolytic cleavage of 43 amino acids from the pro-segment of pro-renin. Many enzymes have been proposed to be capable of activating pro-renin. These include cathepsin B (Wang et al., “Expression of Monocyte Chemotactic Protein and Interleukin-8 by Cytokine-Activated Human Vascular Smooth Muscle Cells,” Arterioscler Thromb 11(5):1166-1174 (1991), which is hereby incorporated by reference in its entirety), cathepsin D (Morris et al., “A “Renin-like” Enzymatic Action of Cathepsin D and the Similarity in Subcellular Distributions of “Renin-like” Activity and Cathepsin D in the Midbrain of Dogs,” Endocrinology 103(4):1289-1296 (1978), which is hereby incorporated by reference in its entirety), cathepsin G (Dzau et al., “Human Neutrophils Release Serine Proteases Capable of Activating Prorenin,” Circ Res 60(4):595-601 (1987), which is hereby incorporated by reference in its entirety), tissue kallikrein (Derkx et al., “Activation of Inactive Plasma Renin by Tissue Kallikreins,” J Clin Endocrinol Metab 49(5):765-769 (1979), which is hereby incorporated by reference in its entirety), and kallikrein-like PRECE. In the current study, it was found that kallikrein-like PRECE expression was increased in heart of WT-p90RSK-Tg and STZ-injected diabetic mice. The kallikrein-like PRECEs (e.g., mouse kallikrein 9 (mKLK9) (GenBank Acc. No. NM_{—}010116), mKLK13 (GenBank Acc. No. NM_{—}010116), mKLK22 (GenBank Acc. No. NM_{—}010114), and mKLK26 (GenBank Acc. No. NM_{—}010644), each of which is hereby incorporated by reference in its entirety) cleave pro-renin on the COOH-side of the Arg residue at the Lys-Arg pair of pro-renin (Kim et al., “The Presence of Two Types of Prorenin Converting Enzymes in the Mouse Submandibular Gland,” FEBS Lett 293(1-2):142-144 (1991); Kim et al., “Mouse Submandibular Gland Prorenin-converting Enzyme Is a Member of Glandular Kallikrein Family,” J Biol Chem 266(29):19283-19287 (1991), which are hereby incorporated by reference in their entirety).

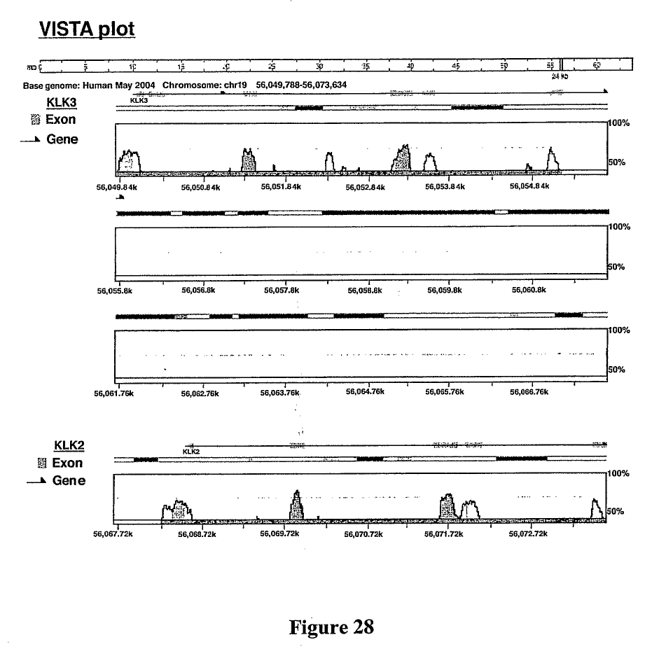

There are 12 mouse kallikrein genes that represent the orthologs of the newly identified human kallikrein genes (KLK4-KLK15). PRECE-1 (mKLK13) and PRECE-2 (mKLK26) have shown 99% sequence similarity, and it has been suggested that PRECE-1 and PRECE-2 represent allelic variants of the same gene (Olsson et al., “Organization and Evolution of the Glandular Kallikrein Locus in Mus Musculus,” Biochem Biophys Res Commun 299(2):305-311 (2002); Diamandis et al., “An Update on Human and Mouse Glandular Kallikreins,” Clin Biochem 37(4):258-260 (2004), which are hereby incorporated by reference in their entirety). An evaluation of the genetic loci in human and mouse shows that the location of PRECE (KLK13) is conserved between the two species, suggesting human KLK13 is orthologous to the mouse PRECE (mKLK13) gene (Olsson et al., “Organization and Evolution of the Glandular Kallikrein Locus in Mus musculus,” Biochem Biophys Res Commun 299(2):305-311 (2002), which is hereby incorporated by reference in its entirety). The conserved region of mouse KLK13 in human from cross-species comparison was also determined by VISTA plot. As shown in FIG. 28, exon 2-5 in both human KLK2 and 3 are highly conserved in mouse KLK13/26 (PRECE) gene. In addition, a highly conserved region between human and mouse were localized in the proximate 0.2-0.3 kb 5′-upstream flanking region of both human KLK2 and 3 genes, as shown in FIG. 28. Based on the VISTA plot analysis, mouse KLK26 (PRECE-2) regions were defined as highly matched to human KLK2 and 3 regions, especially for exons 2 to 5, although there are “dead” sequences (below 50% homology) between human KLK2 and KLK3. It has been reported that KLK2 and KLK3 are the only kallikreins that do not have mouse orthologs among all human glandular kallikrein genes (Diamandis et al., “An Update on Human and Mouse Glandular Kallikreins,” Clin Biochem. 37(4):258-260 (2004), which is hereby incorporated by reference in its entirety). However, this data suggest that mouse KLK13 (PRECE-1) and KLK26 (PRECE-2) can be the mouse gene of human KLK2 and 3. Notably, the proximate 0.2-0.3 kb 5′-upstream flanking region of both human KLK2 and 3 genes is highly conserved, suggesting that these molecules share similar regulatory mechanism. Clark et al have reported that human KLK3 (prostate-specific antigen) expression is regulated by p90RSK activation (Clark et al., “The Serine/Threonine Protein Kinase, p90 Ribosomal S6 Kinase, Is an Important Regulator of Prostate Cancer Cell Proliferation,” Cancer Res 65(8):3108-3116 (2005), which is hereby incorporated by reference in its entirety). These results suggest that human KLK2/3 and mouse KLK13/26 (PRECE) may share a similar regulatory mechanism including p90RSK. Furthermore, it has been reported that plasma pro-renin levels are elevated in human subjects with Fletcher trait (prekallikrein deficiency), also suggesting the important role of KLKs on regulating pro-renin level, not only in mouse but also in human (Derkx et al., “Activation of Inactive Plasma Renin by Tissue Kallikreins,” J Clin Endocrinol Metab 49(5):765-769 (1979); Leckie et al., “Relation Between Renin and Prorenin In Plasma From Hypertensive Patients and Normal People Evidence for Different Renin:Prorenin Ratios,” J Hum Hypertens 9(6):493-496 (1995), which is hereby incorporated by reference in its entirety). The biological roles of human KLK2 and KLK3 have been studied only recently (Diamandis et al., “Human Tissue Kallikreins: a Family of New Cancer Biomarkers,” Clin Chem 48(8):1198-1205 (2002), which is hereby incorporated by reference in its entirety), and further investigation is required, especially to determine the physiological relevance in regulating RAS activity.

Increasing evidence suggests the importance of circulating pro-renin levels and subsequent internalization of pro-renin into cardiac cells, which may play a key role in the process of cardiac damage by RAS (Danser et al., “Prorenin, Renin, Angiotensinogen, and Angiotensin-converting Enzyme in Normal and Failing Human Hearts. Evidence for Renin Binding,” Circulation 96(1):220-226 (1997); Peters et al., “Functional Significance of Prorenin Internalization In the Rat Heart,” Circ Res 90(10): 1135-1141 (2002), which are hereby incorporated by reference in their entirety). Therefore, the induction of PRECE in WT-p90RSK-Tg and diabetic mice may enhance this process and decrease cardiac function after I/R. In support of this, there have been reports that high glucose increases intracellular renin activity by increasing the rate of conversion of pro-renin to active rennin (Vidotti et al., “High Glucose Concentration Stimulates Intracellular Renin Activity and Angiotensin II Generation In Rat Mesangial Cells,” Am J Physiol Renal Physiol 286(6):F1039-1045 (2004), which is hereby incorporated by reference in its entirety). The strong predictive power of plasma pro-renin level, but not renin, for detecting risk of diabetic complications has been reported (Luetscher et al., “Prorenin and Vascular Complications of Diabetes,” Am J Hypertens 2(5 Pt 1):382-386 (1989), which is hereby incorporated by reference in its entirety). This increase of cardiac PRECE may explain the phenomenon described herein, i.e., the p90RSK-dependent PRECE induction in diabetic heart as well as the rapid reduction of angiotensinogen level in WT-p90RSK-Tg mice hearts after KH buffer reperfusion. In addition, the potential benefits of renin inhibitors for diabetic complications has been proposed (Fisher et al., “Renin Inhibition: What Are the Therapeutic Opportunities?” J Am Soc Nephrol 16(3):592-599 (2005), which is hereby incorporated by reference in its entirety). The finding of PRECE induction in diabetic heart may add a novel rationale and therapeutic opportunities of renin inhibitors in preventing cardiac complications in diabetes.

To determine the role of p90RSK activation in the hearts, transgenic (Tg) mice with cardiac specific overexpression of wild type p90RSK (WT-p90RSK-Tg), and transgenic mice exhibiting overexpression of a dominant negative form of p90RSK (DN-p90RSK-Tg) were generated. It was found that expression of pro-renin converting enzyme (PRECE) is specifically up-regulated in WT-p90RSK-Tg mice compared with non-transgenic littermates control mice by analyzing 2D gel image integrated with MALDI-TOF mass spectrometry. Both cardiac p90RSK activation and PRECE expression were significantly increased in diabetic mice induced by streptozotocin (STZ), and this PRECE induction was completely abolished in DN-p90RSK-Tg mice. Furthermore, after 8-10 months of age WT-p90RSK-Tg developed cardiac dysfunction with increased interstitial fibrosis and hypertrophied cardiomyocytes mimicking diabetic cardiomyopathy (Bell DS, “Diabetic Cardiomyopathy. A Unique Entity or a Complication of Coronary Artery Disease?” Diabetes Care 18(5):708-714 (1995), which is hereby incorporated by reference in its entirety). Thus, p90RSK-induces PRECE and subsequent RAS activation in the heart may present a new mechanism to regulate cardiac function, especially in the diabetic heart.

Although preferred embodiments have been depicted and described in detail wherein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A transgenic non-human animal comprising a transgene encoding a mutant p90 ribosomal S6 kinase (p90RSK) that is rendered kinase inactive for S703 phosphorylation of NHE1.

2. The transgenic non-human animal according to claim 1, wherein the mutant p90RSK is a K94A/K447A mutant of wild type p90RSK.

3. The transgenic non-human animal according to claim 1, wherein the transgenic non-human animal expresses the mutant p90RSK in one or more of cardiac muscle cells, smooth muscle cells, skeletal muscle cells, and neuronal cells.

4. The transgenic non-human animal according to claim 1, wherein the animal comprises somatic and germ cells that comprise the transgene.

5. The transgenic non-human animal according to claim 1, wherein the transgenic animal is a somatic mosaic.

6. The transgenic non-human animal according to claim 1, wherein the transgenic animal is a mouse.

7. The transgenic non-human animal of claim 1, wherein said transgenic non-human animal is fertile and transmits said transgene to its offspring.

8. An isolated, recombinant cell comprising a transgene encoding a mutant p90 ribosomal S6 kinase (p90RSK) that is rendered kinase inactive for S703 phosphorylation of NHE1.

9. A method of generating the transgenic non-human animal of claim 1, said method comprising:
introducing a transgene comprising a nucleotide sequence encoding a mutant p90RSK gene operably linked to a nucleic acid promoter into a non-human animal fertilized oocyte;
allowing said fertilized oocyte to develop into an embryo;
transferring said embryo into a pseudopregnant female non-human animal;
allowing said embryo to develop to term, and
identifying said transgenic non-human animal.

10. The method according to claim 9, wherein said identifying comprises confirming that the transgenic non-human animal encodes the mutant p90 ribosomal S6 kinase (p90RSK) that is rendered kinase inactive for S703 phosphorylation of NHE1.

11. The method according to claim 10, wherein said identifying further comprises that the mutant p90RSK is a K94A/K447A mutant of wild type p90RSK.

12. A method of treating an individual to inhibit reperfusion damage following an ischemic event, said method comprising:
administering to an individual an agent that inhibits p90 ribosomal S6 kinase (p90RSK)-induced activation of NHE1, thereby inhibiting activated NHE1-induced reperfusion damage associated with the ischemic event.

13. The method according claim 12, wherein the agent inhibits p90RSK-induced activation of NHE1 without altering basal Na⁺/H⁺ exchange activity in the subject.

14. The method according to claim 12, wherein the agent inhibits p90RSK phosphorylation of NHE1 S703.

15. The method according to claim 12, wherein the agent accelerates dephosphorylation of NHE1 S703.

16. The method according to claim 12, wherein the agent accelerates the dissociation of 14-3-3 from phosphorylated NHE1 S703.

17. The method according claim 12, wherein the ischemic event is a heart attack, acute coronary syndrome, coronary artery bypass surgery, stroke, gastrointestinal ischemia, and peripheral vascular disease.

18. The method according claim 12, wherein said administering is oral, intradermal, intramuscular, intraperitoneal, intravenous, subcutaneous, or intranasal.

19. The method according claim 12, wherein said administering occurs at the time of presentation of the ischemic event.

20. The method according claim 12, wherein said administering occurs prior to presentation of the ischemic event.

21. The method according claim 12, wherein said administering occurs concurrently with the ischemic event.

22. The method according to claim 12, wherein the individual is a mammal.

23. The method according to claim 22, wherein the mammal is human.

24. A method of identifying an agent capable of inhibiting p90 ribosomal S6 kinase (p90RSK)-induced activation of NHE1, said method comprising:
providing a cell culture comprising cells that express p90RSK and NHE1;
treating the cells with an agent to be tested;
exposing the cells to an agonist that normally causes p90RSK-induced activation of NHE1; and
determining the level of p90RSK-induced activation of NHE1 in the treated cells, wherein a reduction in the level of p90RSK-induced activation of NHE1, as compared to untreated cells, indicates efficacy of the agent.

25. The method according to claim 24, wherein said exposing precedes said treating.

26. The method according to claim 24, wherein said exposing follows said treating.

27. The method according to claim 24, wherein said exposing and said treating are concurrent.

28. The method according to claim 24, wherein said exposing comprises adding a reactive oxygen species to the cell culture.

29. The method according to claim 28, wherein the reactive oxygen species is H₂O₂, a molecule that generates H₂O₂, or other reactive oxygen species.

30. The method according to claim 24, wherein said determining comprises measuring H⁺ efflux from the cells in the cell culture.

31. The method according to claim 24, wherein said determining comprises measuring the binding of 14-3-3 proteins to NHE1 in the cells in the cell culture.

32. The method according to claim 24, wherein said determining comprises measuring the S703 phosphorylation of NHE1 in the cells in the cell culture.

33. The method according to claim 24, wherein said determining comprises measuring the S703 dephosphorylation of NHE1 in the cells in the cell culture.

34. The method according to claim 24, wherein said determining comprises measuring the NHE1 S703 phosphorylation using an antibody specific to phosphorylated NHE1 S703.

35. The method according to claim 24, wherein said determining comprises measuring the changes in intracellular pH in the cells of the cell culture.

36. The method according to claim 24, wherein said determining comprises measuring the changes in sodium fluxes in the cells of the cell culture.

37. The method according to claim 24, wherein the cells comprise cells that undergo functional derangement and cell death in response to ischemia/reperfusion, reactive oxygen species or oxidative stress.

38. The method according to claim 37, wherein the cells are selected from the group consisting of cardiac muscle cells, smooth muscle cells, skeletal muscle cells, neuronal cells, or combinations thereof.

39. A method of identifying an agent that modulates ischemic reperfusion (I/R) injury resulting from an ischemic event a transgenic non-human animal whose genome comprises a transgene encoding a mutant p90 ribosomal S6 kinase (p90RSK) that is rendered kinase inactive for S703 phosphorylation of NHE1, said method comprising:
providing a transgenic non-human animal whose genome comprises a transgene encoding a mutant p90 ribosomal S6 kinase (p90RSK) that is rendered kinase inactive for S703 phosphorylation of NHE1 exposing the transgenic non-human animal to conditions effective to produce an ischemic event in the transgenic non-human animal;
administering to the transgenic non-human animal an agent to be tested; and
determining whether the agent modulates the ischemic reperfusion injury resulting from the ischemic event in the transgenic non-human animal.

40. The method according to claim 39, wherein said modulating is an increase or decrease in ischemic reperfusion injury resulting from the ischemic event.

41. The method according claim 39, wherein said administering is oral, intradermal, intramuscular, intraperitoneal, intravenous, subcutaneous, or intranasal.

42. The method according to claim 39, wherein said administering precedes said exposing.

43. The method according to claim 39, wherein said administering follows said exposing.

44. The method according to claim 39, wherein said administering and said exposing are concurrent.

45. The method according to claim 39, wherein the transgene encodes a K94A/K447A mutant of wild type p90RSK.

46. A transgenic non-human animal comprising a transgene that encodes for cardiac-specific overexpression of wild type p90RSK compared to a non-transgenic animal.

47. The transgenic non-human animal according to claim 46, wherein the animal comprises somatic and germ cells that comprise the transgene.

48. The transgenic non-human animal according to claim 46, wherein the transgenic animal is a somatic mosaic.

49. The transgenic non-human animal according to claim 46, wherein the animal is a mouse.

50. The transgenic non-human animal according to claim 46, wherein the transgenic non-human animal further comprises upregulated pro-renin converting enzyme (PRECE) expression in cardiomyocytes compared to a non-transgenic non-human animal.

51. The transgenic non-human animal according to claim 50, wherein the transgenic non-human animal is model for ischemic reperfusion injury (I/R) related to pro-renin converting enzyme (PRECE) expression in the transgenic non-human animal.

52. The transgenic non-human animal according to claim 50, wherein the transgenic non-human animal is model for diabetic cardiomyopathy or renal ischemia.

53. An isolated, recombinant cell comprising a transgene that encodes for animal of cardiac-specific over expression of wildtype p90RSK.

54. A method of generating the transgenic non-human animal of claim 46, comprising:
introducing a transgene comprising a nucleotide sequence encoding a wild type a p90RSK nucleic acid molecule operably linked to an α-MHC promoter into a fertilized transgenic non-human animal oocyte;
allowing said fertilized oocyte to develop into an embryo;
transferring said embryo into a pseudopregnant female transgenic non-human animal;
allowing said embryo to develop to term, and
identifying said transgenic non-human animal.

55. The method according to claim 54, wherein the transgenic non-human animal is a rodent.

56. The method according to claim 55, wherein the transgenic non-human animal is a mouse.

57. The method according to claim 54, wherein said identifying comprises confirming that the transgenic non-human animal overexpresses p90RSK in cardiomyocytes compared to a non-transgenic non-human animal.

58. A method of treating an individual to inhibit ischemia reperfusion injury associated with an ischemic event, said method comprising:
administering to an individual an effective amount of an agent that inhibits p90 ribosomal S6 kinase (p90RSK)-induced activation of pro-renin converting enzyme (PRECE), thereby inhibiting ischemia reperfusion injury associated with an ischemic event.

59. The method according to claim 58, wherein the agent inhibits p90 ribosomal S6 kinase (p90RSK)-induced activation of pro-renin converting enzyme (PRECE) by inhibiting the expression of PRECE in the individual.

60. The method according to claim 59, wherein the agent inhibits p90 ribosomal S6 kinase (p90RSK)-induced activation of pro-renin converting enzyme (PRECE) by inhibiting PRECE enzyme activity.

61. The method according to claim 58, wherein the PRECE is kallikrein-like PRECE.

62. The method according to claim 61, wherein the kallikrein-like PRECE is selected from the group consisting of mKLK9, mKLK13, mKLK22, mKLK26, and an orthologue thereof.

63. The method according to claim 62, wherein the kallikrein-like PRECE is a human orthologue.

64. The method according claim 58, wherein the ischemic event is a heart attack, acute coronary syndrome, coronary artery bypass surgery, stroke, gastrointestinal ischemia, peripheral vascular disease or renal ischemia.

65. The method according to claim 58 wherein the individual has diabetes mellitus.

66. The method according claim 58, wherein said administering occurs at the time of presentation of the ischemic event.

67. The method according claim 58, wherein said administering occurs prior to presentation of the ischemic event.

68. The method according claim 58, wherein said administering occurs concurrently with the ischemic event.

69. The method according to claim 58, wherein the individual is a mammal.

70. The method according to claim 69, wherein the mammal is human.

71. A method of identifying an agent that modulates ischemic reperfusion injury resulting from an ischemic event in a transgenic non-human animal whose genome comprises a transgene encoding for cardiac-specific overexpression of wild type p90 ribosomal S6 kinase (p90RSK), said method comprising:
exposing the transgenic non-human animal to conditions effective to produce an ischemic event in the transgenic non-human animal;
administering to the transgenic non-human animal an agent to be tested; and
determining whether the agent modulates the ischemic reperfusion (I/R) injury resulting from the ischemic event in the transgenic non-human animal.

72. The method according to claim 71, wherein said modulating is an increase or decrease in ischemic reperfusion injury resulting from the ischemic event.

73. The method according claim 71, wherein said administering is oral, intradermal, intramuscular, intraperitoneal, intravenous, subcutaneous, or intranasal.

74. The method according to claim 71, wherein said administering precedes said exposing.

75. The method according to claim 71, wherein said administering follows said exposing.

76. The method according to claim 71, wherein said administering and said exposing are concurrent.

77. The method according to claim 71, wherein the transgenic non-human animal is the transgenic non-human animal according to claim 46.

78. An isolated nucleic acid molecule encoding a mutant p90 ribosomal S6 kinase (p90RSK), wherein the mutant p90RSK is a K94A/K447A mutant of the wild type p90RSK amino acid sequence.

79. The nucleic acid molecule according to claim 78, wherein the mutant p90RSK encodes an inactive kinase.

80. The nucleic acid molecule according to claim 78, wherein the nucleic acid molecule encodes a protein having an amino acid sequence of SEQ ID NO: 1.

81. A nucleic acid construct comprising:
the nucleic acid molecule according to claim 78, and
5′ and 3′ regulatory regions operably linked to the nucleic acid molecule to allow expression of the nucleic acid molecule

82. The nucleic acid construct according to claim 81, wherein the 5′ regulatory region is a tissue-specific expression promoter.

83. The nucleic acid construct according to claim 82, wherein the tissue-specific expression promoter is specific for cardiac tissue.

84. The nucleic acid construct according to claim 83, wherein the promoter is the promoter region of α-myosin heavy chain.

85. An expression system comprising:
the nucleic acid construct according to claim 81.

86. A host comprising the nucleic acid construct according to claim 81, wherein the host is a bacterial cell, a virus, or a mammalian cell.

87. A nucleic acid construct comprising:
a nucleic acid molecule encoding a wild-type p90RSK protein;
a 5′ regulatory region, operably linked to the nucleic acid molecule, wherein the 5′ regulatory region is a tissue-specific expression promoter; and
a 3′ regulatory region operably linked to the nucleic acid molecule to allow expression of the nucleic acid molecule.

88. The nucleic acid construct according to claim 87, wherein the tissue-specific expression promoter is specific for cardiac tissue.

89. The nucleic acid construct according to claim 88, wherein the promoter is the promoter region of α-myosin heavy chain.

90. The nucleic acid construct according to claim 89, wherein the nucleic acid molecule is expressed specifically in cardiomyocytes.

91. An expression system comprising:
the nucleic acid construct according to claim 87.

92. A method of identifying an agent capable of inhibiting p90 ribosomal S6 kinase (p90RSK)-kinase activity on a substrate, said method comprising:
providing a cell culture comprising cells expressing p90RSK;
treating the cells with an agent to be tested; and
determining the level of p90RSK-kinase activity on a substrate in the treated cells, wherein a reduction in the level of p90RSK-kinase activity on a substrate, as compared to untreated cells, indicates efficacy of the agent.

93. The method according to claim 92, wherein said exposing precedes said treating.

94. The method according to claim 92, wherein said exposing follows said treating.

95. The method according to claim 92, wherein said exposing and said treating are concurrent.

96. The method according to claim 92, wherein said exposing precedes said treating.

97. The method according to claim 92, wherein the substrate is PRECE.


SEQUENCE LISTING (12 sequences)

SEQ ID NO: 1 - protein, 735 aa, Rattus norvegicus
MPLAQLKEPWPLMELVPLDPENGQASGEEAGLQPSKDEGILKEISITHHVKAGSEKADPSHFELLKVLGQGSFGKVFLVRKVTRPDNGHLYAMKVLKKATLKVRDRVRTKMERDILADVNHPFVVKLHYAFQTEGKLYLILDFLRGGDLFTRLSKEVMFTEEDVKFYLAELALGLDHLHSLGIIYRDLKPENILLDEEGHIKLTDFGLSKEAIDHEKKAYSFCGTVEYMAPEVVNRQGHTHSADWWSYGVLMFEMLTGSLPFQGKDRKETMTLILKAKLGMPQFLSTEAQSLLRALFKRNPANRLGSGPDGAEEIKRHIFYSTIDWNKLYRREIKPPFKPAVAQPDDTFYFDTEFTSRTPRDSPGIPPSAGAHQLFRGFSFVATGLMEDDSKPRATQAPLHSVVQQLHGKNLVFSDGYIVKETIGVGSYSVCKRCVHKATNMEYAVKVIDKSKRDPSEEIEILLRYGQHPNIITLKDVYDDSKHVYLVTELMRGGELLDKILRQKFFSEREASFVLYTISKTVEYLHSQGVVHRDLKPSNILYVDESGNPECLRICDFGFAKQLRAENGLLMTPCYTANFVAPEVLKRQGYDEGCDIWSLGVLLYTMLAGYTPFANGPSDTPEEILTRISSGKFTLSGGNWNTVSETAKDLVSKMLHVDPHQRLTAKQVLQHPWITQKDKLPQSQLSHQDLQLVKGGMAATYSALSSSKPTPQLKPIESSILAQRRVRKLPSTTL

SEQ ID NO: 2 - protein, 735 aa, Rattus norvegicus mutant p90RSK-1
MPLAQLKEPWPLMELVPLDPENGQASGEEAGLQPSKDEGILKEISITHHVKAGSEKADPSHFELLKVLGQGSFGKVFLVRKVTRPDNGHLYAMAVLKKATLKVRDRVRTKMERDILADVNHPFVVKLHYAFQTEGKLYLILDFLRGGDLFTRLSKEVMFTEEDVKFYLAELALGLDHLHSLGIIYRDLKPENILLDEEGHIKLTDFGLSKEAIDHEKKAYSFCGTVEYMAPEVVNRQGHTHSADWWSYGVLMFEMLTGSLPFQGKDRKETMTLILKAKLGMPQFLSTEAQSLLRALFKRNPANRLGSGPDGAEEIKRHIFYSTIDWNKLYRREIKPPFKPAVAQPDDTFYFDTEFTSRTPRDSPGIPPSAGAHQLFRGFSFVATGLMEDDSKPRATQAPLHSVVQQLHGKNLVFSDGYIVKETIGVGSYSVCKRCVHKATNMEYAVAVIDKSKRDPSEEIEILLRYGQHPNIITLKDVYDDSKHVYLVTELMRGGELLDKILRQKFFSEREASFVLYTISKTVEYLHSQGVVHRDLKPSNILYVDESGNPECLRICDFGFAKQLRAENGLLMTPCYTANFVAPEVLKRQGYDEGCDIWSLGVLLYTMLAGYTPFANGPSDTPEEILTRISSGKFTLSGGNWNTVSETAKDLVSKMLHVDPHQRLTAKQVLQHPWITQKDKLPQSQLSHQDLQLVKGGMAATYSALSSSKPTPQLKPIESSILAQRRVRKLPSTTL

SEQ ID NO: 3 - DNA, 3131 bp, Rattus norvegicus wild-type p90RSK-1
cggcgcggcggacggcccagccagagcgcgaggggctggggggcgtgcgggggtatcggtgcagcagcaaggaccccggggcccagaggcggcacagcccggggccgcccggaggagcgcgggcggtccggcggcggcgcgatgccgctcgcccagctcaaggaaccctggccgctcatggagctggtgccgctggacccggagaatggacaggcttcaggggaagaagctggacttcagccatccaaggatgagggcatcctcaaggagatctctatcacacaccacgtcaaggcaggctctgagaaggctgatccatcccattttgagctcctcaaggttctgggccaaggatcctttggcaaagtcttcctggtacgcaaggtcacccggcctgacaatgggcacttgtatgccatgaaagtattaaagaaggccacgctgaaagtgcgtgaccgtgttcggaccaagatggagagagacatcctagctgacgtgaaccaccccttcgtagtgaaactgcactatgccttccagaccgagggcaagctctatcttattctggactttctgcgtggtggagacctgttcacacgactctcaaaggaggttatgtttacagaggaggatgtgaagttttacctggctgagctggcactgggcctggaccacctgcacagcttgggcatcatttacagagacctcaagcctgagaatatccttttggatgaggagggccacatcaaactcactgactttggcctgagcaaggaggccattgaccacgaaaagaaggcctattccttctgcgggaccgtggagtacatggcgcccgaggttgtcaaccgccagggccacacccacagtgcagattggtggtcctatggggtgttgatgtttgagatgctgacgggctccctgcccttccaggggaaggaccggaaggagaccatgaccttgattttgaaggcaaagctaggcatgccccagtttctgagcacggaagcccagagcctcctgcgggccctgttcaagaggaatcctgccaaccggcttggctcaggccccgatggggctgaggaaattaagagacatatcttctactctaccattgactggaataagctctaccgccgtgagatcaagccacctttcaagcccgctgtggcccagccggatgacaccttctactttgataccgagttcacgtcacgcacacccagggattcgccgggcatcccccccagtgctggtgcccatcagctcttccgtggcttcagcttcgtggccaccggtctgatggaggatgacagcaagcctcgggccacccaggctccgctgcactcggtggtacagcaactccacgggaagaacttggttttcagcgatggctacatagtaaaggagacgatcggcgtgggctcctactctgtgtgtaagcgctgtgtccacaaggccaccaacatggagtacgcagtcaaagtaatcgacaaaagcaaaagagatccctccgaagagatcgagattcttctgcggtatggacagcaccccaacatcatcaccctgaaagatgtgtatgacgacagtaagcacgtatacctggtgacagagctgatgaggggcggggagctgctggataagatcctacggcagaaattcttctcagagcgggaggccagcttcgtcctgtacaccatcagcaagactgtggaatacttgcactcccaaggggtcgtccacagggacctcaaacccagtaacatcctgtatgtggatgagtctgggaaccccgaatgcctacgaatatgcgactttggctttgccaagcagctacgggctgagaacgggcttctcatgacaccttgctacacagccaactttgtggcacctgaggtgctgaagcgtcagggctacgatgaaggctgtgacatatggagcctgggcgttctgctgtacacgatgctggcaggatacactccatttgccaatgggcccagtgataccccagaggagatcctcacccggatcagcagtgggaagttcaccctcagtgggggaaactggaacacggtttcagagacagccaaggacttagtatctaagatgctgcatgtggacccccaccagcgcctcacagccaaacaggttctgcagcacccgtggatcacccagaaagacaagctcccccagagccagttgtcccaccaagacctgcagcttgtgaaggggggcatggcagctacatattctgcactcagtagctccaaacccaccccccagctcaagccaatcgagtcgtccatcctggcccagcggcgggtgaggaagctgccatccaccaccctgtgaacgacagtgcgagcaaactcctctgaggcagagtccttccagagggagcaagcctgagtcacagaccaagtggaatggagtcctaaaggaagcaactagcccagctcacccgtgcgggtgtgaagtgccttcctccccaggacgggctcttctgggctcaggctccattgtgtgaaatccactcactgtacaaactatttttaagaaaggaaaaagaaaaaatgacatcatttaccatggattttttttttacaagatccatttggctttttggccattgcagtcccaggaggaacacccagtcccatgtgtggccaagactcccgtgatagctttgggactccgcccctctgttggtcaaggagccatctgcacccgcctccgagcacgttcggcgttgcctctcagagttgtcgactggctcctcagcagaacttggtgtccccagccatctctttttccattctgttctggggttctcgaaccactttctgctaagagcccgggactccaccctgtgcagctcttggctcaggcaccagcatccacagcgccccatgcgcagttgggcccctgcagtcagaacgggcagccccgtggagaggagacggagagcactttttgggagacttcctgttctgccactggacagagttcacaggagaccagggaggtagtccacgggggatgagggctttttccctttcctcctcagctggtaactcagggttcatctgtccaaggcctttctaataaacctacagtccagtcaaaaaaaaaaa

SEQ ID NO: 4 - DNA, 32 bp, Artificial Sequence
  Description of Artificial Sequence Oligonucleotide
ttagcaaacctcaggcacccttaccccacata

SEQ ID NO: 5 - DNA, 33 bp, Artificial Sequence
  Description of Artificial Sequence Oligonucleotide
taggatgtctctccatcttggtccgaacacggt

SEQ ID NO: 6 - DNA, 21 bp, Artificial Sequence
  Description of Artificial Sequence Oligonucleotide
atgtcgaccagtatgaggttt

SEQ ID NO: 7 - DNA, 20 bp, Artificial Sequence
  Description of Artificial Sequence Oligonucleotide
tgactttctgtaggtagact

SEQ ID NO: 8 - DNA, 20 bp, Artificial Sequence
  Description of Artificial Sequence Oligonucleotide
ctgctggagctgataagaga

SEQ ID NO: 9 - DNA, 19 bp, Artificial Sequence
  Description of Artificial Sequence Oligonucleotide
tgcccaaagcagcttgaga

SEQ ID NO: 10 - DNA, 20 bp, Artificial Sequence
  Description of Artificial Sequence Oligonucleotide
gagaagatgccggtagaaga

SEQ ID NO: 11 - DNA, 20 bp, Artificial Sequence
  Description of Artificial Sequence Oligonucleotide
aagcactgccgtctctcaga

SEQ ID NO: 12 - protein, 261 aa, Mus sp.
MWFLILFPALSLGGIDAAPPLQSRVVGGFNCEKNSQPWQVAVYYQKEHICGGVLLDRNWVLTAAHCYVDQYEVWLGKNKLFQEEPSAQHRLVSKSFPHPGFNMSLLMLQTTPPGADFSNDLMLLRLSKPADITDVVKPIALPTKEPKPGSTCLASGWGSITPTRWQKSDDLQCVFITLLPNENQAKVYLQKVTDVMLCAGEMGGGKDTCAGDSGGPLICDGILQGTTSNGPEPCGKPGVPAIYTNLIKFNSWIKDTMMKNA